(19) United States
(12) Patent Application Publication
(10) Pub. No.: US 2022/0173318 A1
(43) Pub. Date: Jun. 2, 2022

(54) ORGANIC LIGHT-EMITTING DEVICE

(71) Applicant: LG CHEM, LTD., Seoul (KR)

(72) Inventors: Jihye KIM, Daejeon (KR); Sung Kil HONG, Daejeon (KR); Wanpyo HONG, Daejeon (KR); Sung Jae LEE, Daejeon (KR); Sujeong GEUM, Daejeon (KR)

(21) Appl. No.: 17/442,762

(22) PCT Filed: May 15, 2020
(86) PCT No.: PCT/KR2020/006408

§ 371 Date: Sep. 24, 2021

(30) Foreign Application Priority Data
May 15, 2019 (KR) 10-2019-0057177

(51) Int. Cl. H01L 51/00

(52) U.S. Cl. CPC H01L 51/008; H01L 51/006; H01L 51/0058; H01L 51/0072; H01L 51/0056; H01L 51/5072; H01L 51/0073; H01L 51/0061; H01L 51/0054; H01L 51/0067; H01L 51/0094

(57) ABSTRACT

Provided is an organic light emitting device including a positive electrode, a negative electrode, and an organic material layer provided between the positive electrode and the negative electrode, wherein the organic material layer comprises a hole transport material having a HOMO absolute value of 4.30 eV to 4.60 eV, and a reversibility value (Iᵣ/I_{f}) of 0.83 or higher within an oxidation range at a scan rate of 100 mV/s, or the organic material layer comprises an electron blocking material having a reversibility value (Iᵣ/I_{f}) of more than 0.5 within an oxidation range at a scan rate of 100 mV/s, or the organic material layer comprises an electron transport material having a LUMO absolute value of 2.60 eV to 2.90 eV, and a reversibility value (Iᵣ/I_{f}) larger than [4.96−1.535×(the LUMO absolute value)] within a reduction range at a scan rate of 100 mV/s.

### CROSS-REFERENCE TO RELATED APPLICATIONS

This application is a National Stage Application of International Application No. PCT/KR2020/006408 filed on May 15, 2020, which claims priority to and the benefit of Korean Patent Application No. 10-2019-0057177 filed in the Korean Intellectual Property Office on May 15, 2019, the entire contents of which are incorporated herein by reference.

### TECHNICAL FIELD

The present specification relates to an organic light emitting device.

### BACKGROUND

In general, an organic light emitting phenomenon refers to a phenomenon in which electric energy is converted into light energy by using an organic material. An organic light emitting device using the organic light emitting phenomenon typically has a structure including a positive electrode, a negative electrode, and an organic material layer disposed therebetween. Here, the organic material layer can have a multi-layered structure composed of different materials in many cases in order to improve the efficiency and stability of the organic light emitting device, and can be composed of, for example, a hole injection layer, a hole transport layer, a light emitting layer, an electron transport layer, an electron injection layer, and the like.

In order to improve the performance of the organic light emitting device, studies on the use of an appropriate material in an appropriate organic material layer in the structure of the organic light emitting devices have been continuously conducted.

[Prior Art Documents] (Patent 1) Korean Patent No. 10-1347240

### BRIEF DESCRIPTION

### Technical Problem

The present specification has been made in an effort to provide an organic light emitting material having good performance by evaluating reversibility, that is, electrical stability in the (+) radical and (−) radical states of a material for an organic light emitting device using cyclic voltammetry (CV), and an organic light emitting device including the same.

### Technical Solution

An exemplary embodiment of the present specification provides an organic light emitting device including: a positive electrode; a negative electrode; and an organic material layer provided between the positive electrode and the negative electrode.

In an exemplary embodiment, the organic material layer includes a hole transport material (HT), and the hole transport material (HT) has a HOMO absolute value of 4.30 eV to 4.60 eV, and a reversibility value (Iᵣ/I_{f}) of 0.83 or higher within an oxidation range at a scan rate of 100 mV/s at the time of measuring cyclic voltage current.

In another exemplary embodiment, the organic material layer includes an electron blocking material (EB), and the electron blocking material (EB) has a reversibility value (Iᵣ/I_{f}) of more than 0.5 within an oxidation range at a scan rate of 100 mV/s at the time of measuring cyclic voltage current.

In still another exemplary embodiment, the organic material layer includes a blue light emitting dopant material (BD), and the blue light emitting dopant material (BD) has a LUMO absolute value of 2.40 eV to 2.74 eV, and a reversibility value (Iᵣ/I_{f}) larger than [−23.14+8.458×(the LUMO absolute value)] within a reduction range at a scan rate of 100 mV/s at the time of measuring cyclic voltage current.

In yet another exemplary embodiment, the organic material layer includes an electron transport material (ET), and the electron transport material (ET) has a LUMO absolute value of 2.60 eV to 2.90 eV, and a reversibility value (Iᵣ/I_{f}) larger than [4.96−1.535×(the LUMO absolute value)] within a reduction range at a scan rate of 100 mV/s at the time of measuring cyclic voltage current.

In still yet another exemplary embodiment, the organic material layer includes a hole blocking material (HB), and the hole blocking material (HB) has both a forward peak and an inverse peak at a scan rate of 100 mV/s at the time of measuring cyclic voltage current within an oxidation range.

In a further exemplary embodiment, the organic material layer includes a blue light emitting host material (BH), and the blue light emitting host material (BH) has a reversibility value (Iᵣ/I_{f}) of [1.34×(the dipole moment)−0.293] or higher within an oxidation range at a scan rate of 500 mV/s and a reversibility value (Iᵣ/I_{f}) of 0.95 or higher within a reduction range at a scan rate of 10 mV/s, at the time of measuring cyclic voltage current.

In another further exemplary embodiment, the organic material layer includes a light emitting host material (EML), and the light emitting host material (EML) has a reversibility value (Iᵣ/I_{f}) of [0.955−0.1786×(a reversibility value (Iᵣ/I_{f}) within an oxidation range)] or higher within a reduction range at a scan rate of 10 mV/s at the time of measuring cyclic voltage current.

In still another further exemplary embodiment, the organic material layer including the hole transport material (HT) further includes an electron blocking material (EB) and has a [(HT Iᵣ/I_{f})−(EB Iᵣ/I_{f})] value of 0.15 or less, the HT Iᵣ/I_{f} is a reversibility value of the hole transport material (HT) within an oxidation range at a scan rate of 100 mV/s, and the EB Iᵣ/I_{f} is a reversibility value of the electron blocking material (EB) within an oxidation range at a scan rate of 100 mV/s. In this case, the hole transport material (HT) and the electron blocking material (EB) are included in different organic material layers, respectively.

In yet another further exemplary embodiment, the organic material layer including the blue light emitting dopant material (BD) further includes a blue light emitting host material (BH) and has a [(the LUMO absolute value of the blue light emitting host material (BH))−(the LUMO absolute value of the blue light emitting dopant material (BD))] value of 0.16 eV to 0.75 eV. In this case, the blue light emitting dopant material (BD) and the blue light emitting host material (BH) are included in the same layer.

In still yet another further exemplary embodiment, the organic material layer including the electron transport material (ET) further includes a hole blocking material (HB) and has a [the LUMO absolute value of the electron transport material (ET)−the LUMO absolute value of the hole blocking material (HB)] of 0.05 eV to 0.3 eV. In this case, the electron transport material (ET) and the hole blocking material (HB) are included in different organic material layers, respectively.

In still yet another further exemplary embodiment, the organic material layer including the light emitting host material (EML) further includes an electron transport material (ET) and has a [(the LUMO absolute value of the light emitting host material (EML))−(the LUMO absolute value of the electron transport material (ET))] value of 0.15 eV to 0.35 eV. In this case, the light emitting host material (EML) and the electron transport material (ET) are included in a different layer, respectively, or included in the same layer.

### Advantageous Effects

The organic light emitting device according to an exemplary embodiment of the present specification includes a material which is excellent in electrical stability in the (+) and (−) radical states of an organic light emitting material. The organic light emitting device composed of the material can have long service life characteristics.

### BRIEF DESCRIPTION OF THE DRAWING

FIG. 1 illustrates an example of an organic light emitting device.

### EXPLANATION OF REFERENCE NUMERALS AND SYMBOLS

101: Substrate
102: Positive electrode
103: Organic material layer
104: Negative electrode

### DETAILED DESCRIPTION

Service life characteristics of an organic light emitting device are affected by the electrical stability in the (+) radical or (−) radical state of a material for an organic light emitting device. In the related art, as a method for evaluating the electrical stability of a material for an organic light emitting device, a method for comparing reduced capacitance using a cyclic voltammetry has been used. However, this method does not measure the electrical stability of the (+) radical or (−) radical of a material for an organic light emitting device.

The present invention establishes a method for comparing the stabilities of the (+) and (−) radicals of a sample by analyzing the graph outline of a cyclic voltammogram measured by a cyclic voltammetry (CV) within an oxidation range and a reduction range, and provides a method for selecting a stable material for an organic light emitting device to be applied to an organic material layer of an organic light emitting device.

Hereinafter, the present specification will be described in detail.

In the present specification, the cyclic voltammogram is measured by a VSP model. Specifically, the cyclic voltammetry (CV), which measures current generated by changing voltage, is used. The voltage of the working electrode is changed from the initial voltage (Eᵢ) to the constant rate (v) (E=Eᵢ−vt, t is time), and the current is measured. In this case, v is called a scan rate.

In the present specification, a peak refers to a point at which the sign of the slope in the graph changes.

In the present specification, the height of a peak refers to a value obtained by subtracting the current value of the baseline from the current value of the corresponding peak in the cyclic voltammogram.

In the present specification, the current value refers to the absolute value of the current in the cyclic voltammogram.

In the present specification, the forward peak refers to the point where the current size is the largest in the forward scan of the cyclic voltammogram. The increased current decreases from the forward peak.

In the present specification, the inverse peak refers to the point where the current size is the largest in the inverse scan of the cyclic voltammogram. The increased current decreases from the inverse peak. In the present specification, the point where the peak appears except for the forward peak and the inverse peak in the cyclic voltammogram refers to as an impurity peak. The region where the impurity peak appears is not limited to the forward scan or the inverse scan. That is, the impurity peak can appear in the forward scan, can appear in the inverse scan, and can appear in both the forward scan and the inverse scan. There can be one or more impurity peaks.

In the present specification, a lowest unoccupied molecular orbital (LUMO) and a highest occupied molecular orbital (HOMO) can be obtained by the cyclic voltammetry.

E(HOMO)=[Vₛₒₗᵥₑₙₜ−(E_{onset ox}−E_{1/2}(solvent)]eV

E(LUMO)=[Vₛₒₗᵥₑₙₜ−(E_{onset red}−E_{1/2}(solvent)]eV

Vₛₒₗᵥₑₙₜ is the energy level of the solvent, E_{1/2} is the half-wave level of the solvent, E_{onset ox} is the level (potential) of the point where the oxidation starts, and E_{onset red} is the level (potential) of the point where the reduction starts.

The HOMO and the LUMO can be measured using an AC3 device even in addition to the cyclic voltammetry (CV), and can also be calculated by a simulation program.

In the present specification, the HOMO or LUMO value to be measured (or calculated) is a value of the measured oxidation potential or reduction potential calibrated by a calibration material ferrocene.

HOMO=4.8−(the oxidation potential of ferrocene−the oxidation potential of a sample)

LUMO=4.8−(the oxidation potential of ferrocene−the reduction potential of a sample)

In the present specification, when the HOMO or LUMO is calculated by a simulation program, a Gaussian program or a Schrodinger program can be used as the simulation program. A time-dependent density functional theory (DFT) tool can be used.

In the present specification, the HOMO or LUMO value measured (or calculated) by AC3 is a value obtained by depositing a material onto an ITO film and then putting the deposited ITO film into an AC3 device to measure a work function.

According to an exemplary embodiment of the present specification, as a method of obtaining the cyclic voltammogram, the cyclic voltammogram is obtained under the conditions of N2 gas and an electrolyte (TBAC: tert-butyl acetate) by preparing a sample in which a target compound is dissolved in dimethylformamide (DMF) at a concentration of 0.003 M. In this case, the cyclic voltammogram is fitted by the EC-lab program and is measured by the VSP model.

In the present specification, the oxidation range refers to a voltage range in which oxidation can occur.

In the present specification, the reduction range refers to a voltage range in which reduction can occur.

In the present specification, blue refers to a light emission color having a maximum light emitting peak of 380 nm to 500 nm.

In the present specification, the dipole moment (D.M) (Debye) was calculated using a quantum chemical calculation program Gaussian 03 manufactured by U.S. Gaussian Corporation, and a density functional theory (DFT) was used and the calculated value of the triplet energy was obtained by the time-dependent-density functional theory (TD-DFT) with respect to a structure optimized using B3LYP as a functional and 6-31G* as a basis function.

In the present specification, “p to q” means greater than or equal to p and less than or equal to q.

In the present specification, it is assumed that the current size of the peak at the time of measuring 2 cycles to 10 cycles changes within 3% of the reference value.

According to an exemplary embodiment of the present specification, a material suitable for an organic material layer of an organic light emitting device is provided by measuring and analyzing the cyclic voltage current of an organic light emitting material.

In an exemplary embodiment of the present specification, the cyclic voltage current of the organic light emitting material can be measured within the oxidation range or reduction range.

In an exemplary embodiment of the present specification, the cyclic voltage current is measured by dissolving an organic light emitting material within the oxidation range or reduction range in an organic solvent.

According to an exemplary embodiment of the present specification, the organic solvent is dimethylformamide (DMF).

In the present specification, the reversibility can be quantified as a value of the following Equation 1. Specifically, the reversibility in the reference scan rate is defined by the following Equation 1:

Reversibility=Iᵣ/I_{f} <Equation 1>

In Equation 1, Iᵣ means the height of the inverse peak, and I_{f} means the height of the forward peak.

The reference scan rate refers to a rate at which the graph outlines can be compared among materials while all the corresponding comparative materials have a forward peak and an inverse peak.

The height of the peak refers to a value obtained by subtracting the current value in the baseline from the current value of the corresponding peak.

Specifically, the height of the peak can be measured from a program which measures the CV.

In the present specification, the oxidation stability is a reversibility value calculated from the cyclic voltammogram obtained within the oxidation range.

In the present specification, the reduction stability is a reversibility value calculated from the cyclic voltammogram obtained within the reduction range.

A material having a high reversible stability (reduction stability) within the reduction range has a stable anion radical state. Therefore, when a material having a high reversible stability within the reduction range is used as a dopant material of a blue light emitting layer, the service life characteristics of the organic light emitting device can be improved.

A material having a high reversible stability (oxidation stability) within the oxidation range has a stable cation radical state. Therefore, when a material having a high reversible stability within the oxidation range is used as a host of the blue light emitting layer, hole transport layer, electron blocking layer, electron transport layer or hole blocking layer material, the service life characteristics of the organic light emitting device can be improved.

The present specification provides an organic light emitting device including an organic material layer. Specifically, the present specification provides an organic light emitting device including: a positive electrode; a negative electrode; and an organic material layer provided between the positive electrode and the negative electrode.

In an exemplary embodiment of the present specification, the organic material layer includes a hole transport material (HT), and the hole transport material (HT) has a HOMO absolute value of 4.30 eV to 4.60 eV, and a reversibility value (Iᵣ/I_{f}) of 0.83 or higher within an oxidation range at a scan rate of 100 mV/s at the time of measuring cyclic voltage current.

The HOMO absolute value of the hole transport material (HT) is calculated by a simulation program. In an exemplary embodiment, the HOMO absolute value of the hole transport material (HT) is calculated by a time-dependent density functional theory (DFT) of a Gaussian program.

In an exemplary embodiment of the present specification, the hole transport material (HT) has a reversibility value (Iᵣ/I_{f}) of 1.2 or lower, preferably 1.0 or lower within an oxidation range at a scan rate of 100 mV/s at the time of measuring cyclic voltage current.

In an exemplary embodiment of the present specification, the hole transport material (HT) is an arylamine compound, and a fluorene compound, a spirobifluorene compound, or a carbazole-based compound.

In an exemplary embodiment of the present specification, the hole transport material (HT) is a compound of the following Formula 1 or 2:

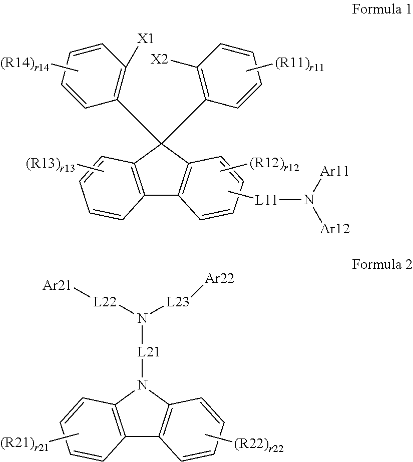

wherein in Formulae 1 and 2:

X1 and X2 are each hydrogen or deuterium, or are directly single-bonded to each other to form a ring;

R11 to R14, R21 and R22 are the same as or different from each other, and are each independently hydrogen, deuterium, a substituted or unsubstituted alkyl group, a substituted or unsubstituted amine group, or a substituted or unsubstituted aryl group, or can be each bonded to an adjacent group to form a substituted or unsubstituted ring;

L11 and L21 to L23 are the same as or different from each other, and are each independently a single bond or a substituted or unsubstituted arylene group;

Ar11, Ar12, Ar21 and Ar22 are the same as or different from each other, and are each independently hydrogen, deuterium, a halogen group, a cyano group, a nitro group, a substituted or unsubstituted silyl group, a substituted or unsubstituted cycloalkyl group, a substituted or unsubstituted aryl group, or a substituted or unsubstituted heteroaryl group;

r11, r13, r14, r21, and r22 are each an integer from 0 to 4, and r12 is an integer from 0 to 3; and

when r11 to r14, r21, and r22 are 2 or higher, the substituents in the parenthesis are the same as or different from each other.

In an exemplary embodiment of the present specification, when X1 and X2 are directly single-bonded to each other to form a ring, the core of Formula 1 includes spirobifluorene.

In an exemplary embodiment of the present specification, R11 to R14 are the same as or different from each other, and are each independently hydrogen, deuterium, an alkyl group having 1 to 6 carbon atoms, or an aryl group having 6 to 30 carbon atoms.

In an exemplary embodiment of the present specification, R11 to R14 are the same as or different from each other, and are each independently hydrogen, deuterium, a methyl group, an ethyl group, a propyl group, a t-butyl group, a phenyl group, a biphenyl group, or a naphthyl group.

In an exemplary embodiment of the present specification, L11 is a single bond or an arylene group having 6 to 30 carbon atoms.

In an exemplary embodiment of the present specification, L11 is a single bond, a phenylene group, a biphenylene group, or a naphthylene group.

In an exemplary embodiment of the present specification, Ar11 and Ar12 are the same as or different from each other, and are each independently an aryl group having 6 to 30 carbon atoms, which is unsubstituted or substituted with an alkyl group having 1 to 10 carbon atoms; or a heteroaryl group having 2 to 30 carbon atoms.

In an exemplary embodiment of the present specification, Ar11 and Ar12 are each the same as or different from each other, and are each independently a phenyl group which is unsubstituted or substituted with an alkyl group having 1 to 6 carbon atoms; a biphenyl group which is unsubstituted or substituted with an alkyl group having 1 to 6 carbon atoms; a terphenyl group which is unsubstituted or substituted with an alkyl group having 1 to 6 carbon atoms; a naphthyl group which is unsubstituted or substituted with an alkyl group having 1 to 6 carbon atoms; a fluorenyl group which is unsubstituted or substituted with an alkyl group having 1 to 6 carbon atoms; a dibenzofuran group; or a dibenzothiophene group.

In an exemplary embodiment of the present specification, Ar11 and Ar12 are the same as or different from each other, and are each independently a phenyl group, a biphenyl group, a terphenyl group, a naphthyl group, a dibenzofuran group, or a dibenzothiophene group.

In an exemplary embodiment of the present specification, L21 to L23 are the same as or different from each other, and are each independently a single bond or an arylene group having 6 to 30 carbon atoms.

In an exemplary embodiment of the present specification, L21 to L23 are the same as or different from each other, and are each independently a single bond, a phenylene group, a biphenylene group, a terphenylene group, or a naphthylene group.

In an exemplary embodiment of the present specification, L22 and L23 are the same as or different from each other, and are each independently a single bond, a phenylene group, a biphenylene group, a terphenylene group, or a naphthylene group.

In an exemplary embodiment of the present specification, L21 is a phenylene group, a biphenylene group, a terphenylene group, or a naphthylene group.

In an exemplary embodiment of the present specification, Ar21 and Ar22 are the same as or different from each other, and are each independently hydrogen, deuterium, a halogen group, a cyano group, a nitro group, an alkylsilyl group having 1 to 30 carbon atoms, an arylsilyl group having 6 to 90 carbon atoms, a cycloalkyl group having 3 to 10 carbon atoms, an aryl group having 6 to 30 carbon atoms, or a heteroaryl group having 2 to 30 carbon atoms.

In an exemplary embodiment of the present specification, Ar21 and Ar22 are the same as or different from each other, and are each independently a cyano group, an alkylsilyl group having 1 to 15 carbon atoms, an arylsilyl group having 6 to 50 carbon atoms, a cycloalkyl group having 3 to 10 carbon atoms, an aryl group having 6 to 30 carbon atoms, or a heteroaryl group having 2 to 30 carbon atoms.

In an exemplary embodiment of the present specification, Ar21 and Ar22 are the same as or different from each other, and are each independently a phenyl group, a biphenyl group, a terphenyl group, a naphthyl group, a dibenzofuran group, or a dibenzothiophene group.

In an exemplary embodiment of the present specification, R21 and R22 are the same as or different from each other, and are each independently hydrogen or deuterium, or can be each bonded to an adjacent group to form a substituted or unsubstituted aromatic hydrocarbon ring.

In an exemplary embodiment of the present specification, R21 and R22 are the same as or different from each other, and are each independently hydrogen or deuterium, or can be each bonded to an adjacent group to form a benzene ring.

In an exemplary embodiment of the present specification, the hole transport material (HT) is selected from the following compounds:

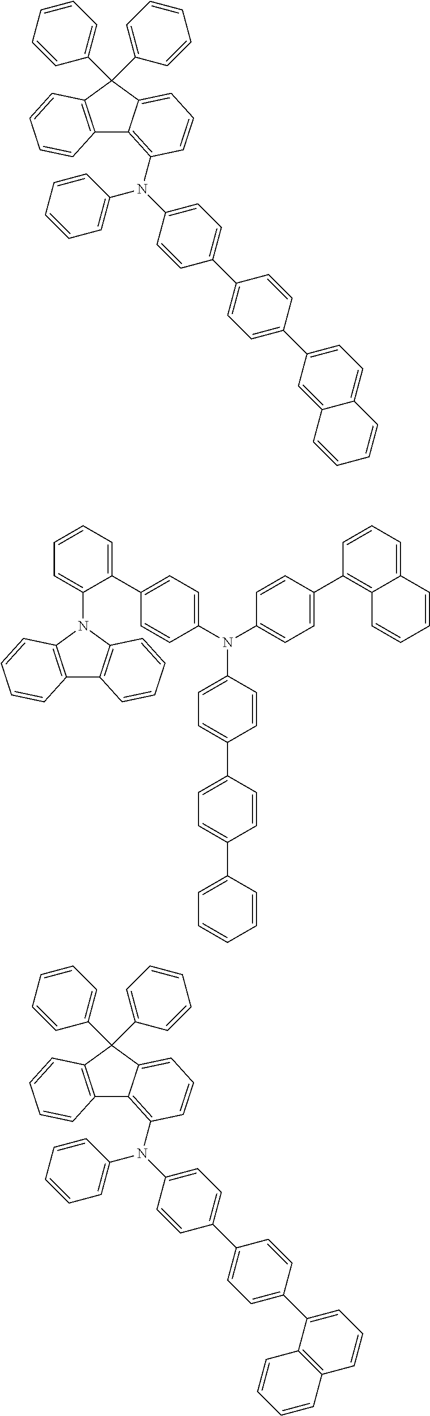 [SMILES: c1ccc(N(c2ccc(-c3ccc(-c4ccc5ccccc5c4)cc3)cc2)c2cccc3c2-c2ccccc2C3(c2ccccc2)c2ccccc2)cc1 ; c1ccc(-c2ccc(-c3ccc(N(c4ccc(-c5ccccc5-n5c6ccccc6c6ccccc65)cc4)c4ccc(-c5cccc6ccccc56)cc4)cc3)cc2)cc1 ; c1ccc(N(c2ccc(-c3ccc(-c4cccc5ccccc45)cc3)cc2)c2cccc3c2-c2ccccc2C3(c2ccccc2)c2ccccc2)cc1]

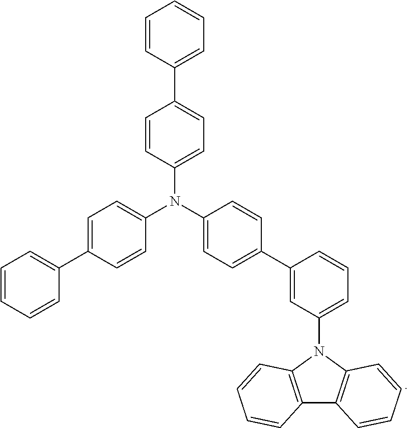 [SMILES: c1ccc(-c2ccc(N(c3ccc(-c4ccccc4)cc3)c3ccc(-c4cccc(-n5c6ccccc6c6ccccc65)c4)cc3)cc2)cc1]

In an exemplary embodiment of the present specification, the organic material layer includes an electron blocking material (EB), and the electron blocking material (EB) has a reversibility value (Iᵣ/I_{f}) of more than 0.5 within an oxidation range at a scan rate of 100 mV/s at the time of measuring cyclic voltage current.

In an exemplary embodiment of the present specification, the electron blocking material (EB) has a reversibility value (Iᵣ/I_{f}) of 0.7 or higher, preferably 0.9 or higher within an oxidation range at a scan rate of 100 mV/s at the time of measuring cyclic voltage current.

In an exemplary embodiment of the present specification, the electron blocking material (EB) has a reversibility value (Iᵣ/I_{f}) of 1.2 or lower, preferably 1.0 or lower within an oxidation range at a scan rate of 100 mV/s at the time of measuring cyclic voltage current.

In an exemplary embodiment of the present specification, the electron blocking material (EB) has a HOMO absolute value of 5.23 eV to 5.42 eV.

The HOMO absolute value of the electron blocking material (EB) is calculated by a simulation program. In an exemplary embodiment, the HOMO absolute value of the electron blocking material (EB) is calculated by a time-dependent density functional theory (DFT) of a Gaussian program.

In an exemplary embodiment of the present specification, the electron blocking material (EB) is an arylamine compound, or a carbazole-based compound.

In an exemplary embodiment of the present specification, the electron blocking material (EB) is the compound of Formula 1 or 2.

In an exemplary embodiment of the present specification, Formulae 1 and 2 of the electron blocking material (EB) are the same as those described in Formulae 1 and 2 of the hole transport material (HT).

In an exemplary embodiment of the present specification, the electron blocking material (EB) is selected from the following compounds:

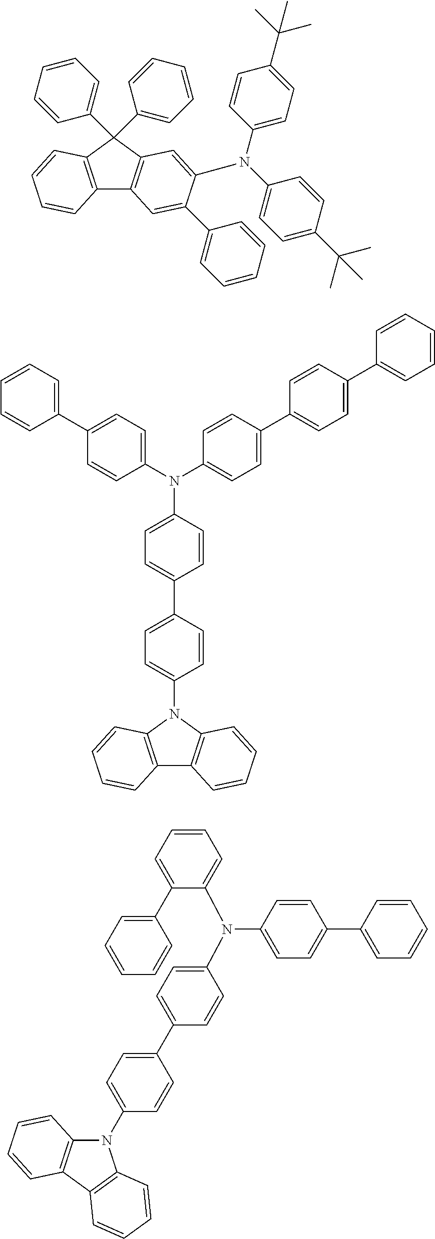

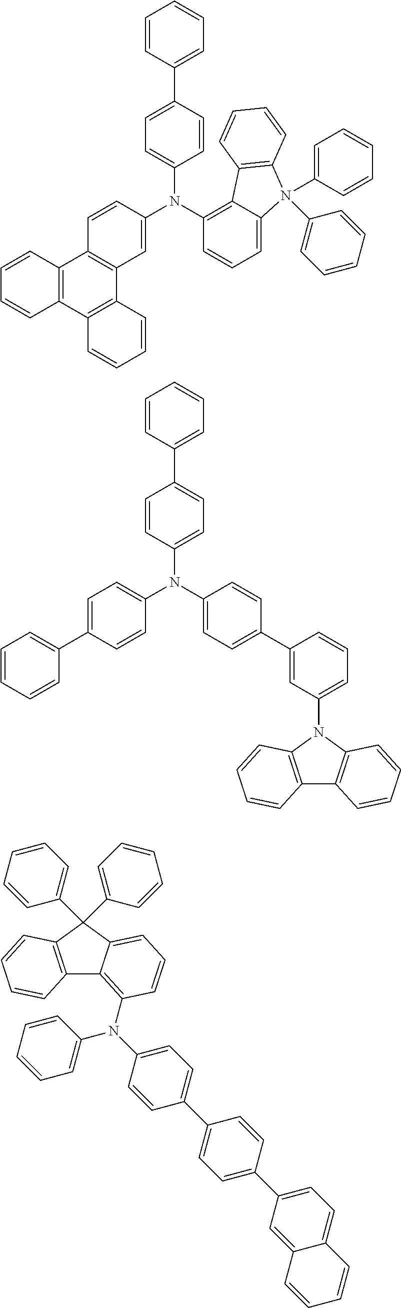

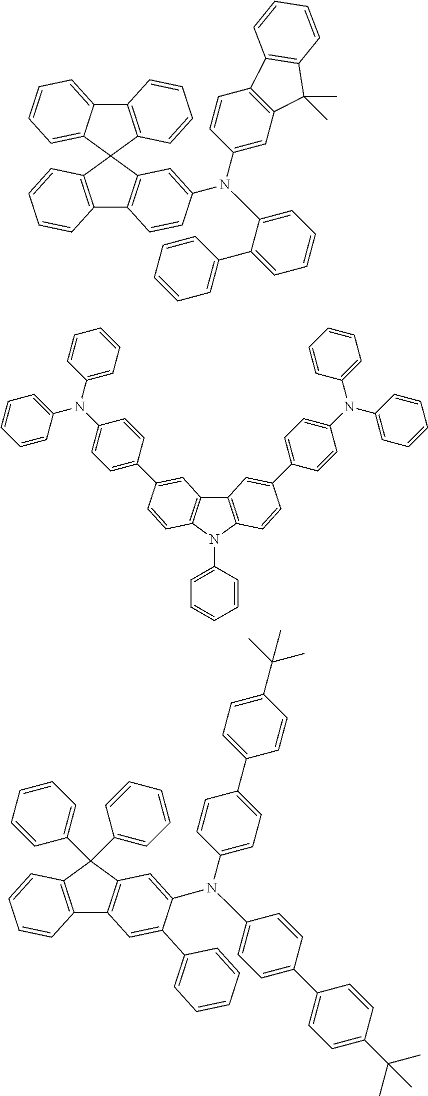

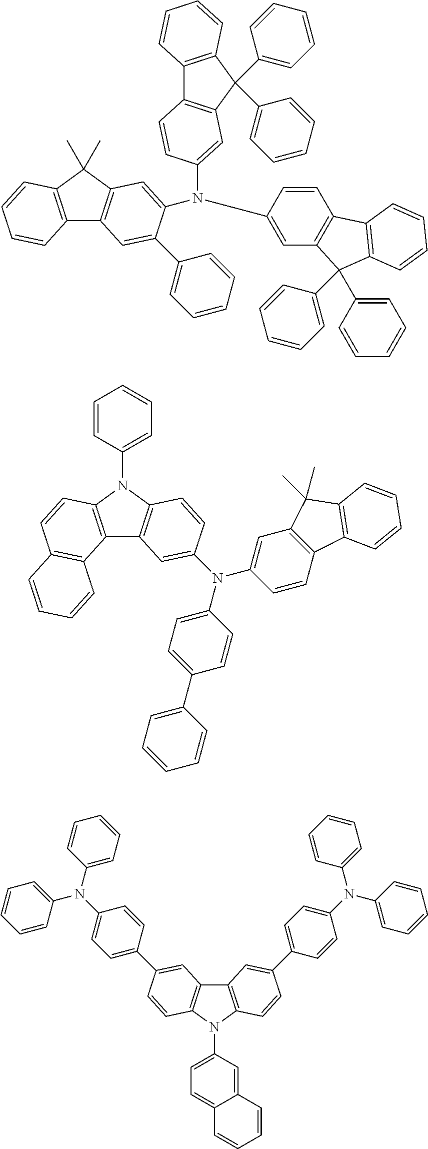 [SMILES: CC1(C)c2ccccc2-c2cc(-c3ccccc3)c(N(c3ccc4c(c3)C(c3ccccc3)(c3ccccc3)c3ccccc3-4)c3ccc4c(c3)C(c3ccccc3)(c3ccccc3)c3ccccc3-4)cc21 ; CC1(C)c2ccccc2-c2ccc(N(c3ccc(-c4ccccc4)cc3)c3ccc4c(c3)c3c5ccccc5ccc3n4-c3ccccc3)cc21 ; c1ccc(N(c2ccccc2)c2ccc(-c3ccc4c(c3)c3cc(-c5ccc(N(c6ccccc6)c6ccccc6)cc5)ccc3n4-c3ccc4ccccc4c3)cc2)cc1]

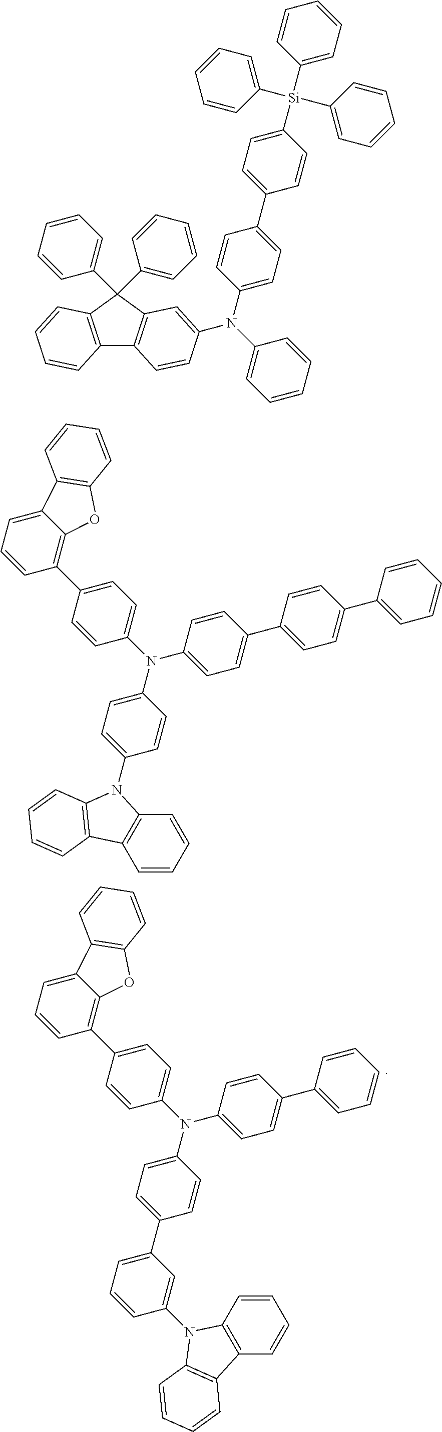

The present specification provides an organic light emitting device including the hole transport material (HT) and the electron blocking material (EB) described above. Specifically, the organic light emitting device includes an organic material layer, and the organic material layer includes a hole transport layer and an electron blocking layer. The hole transport layer includes the above-described hole transport material (HT), and the electron blocking layer includes the above-described electron blocking material (EB). In this case, a value of (HT Iᵣ/I_{f})−(EB Iᵣ/I_{f}) is 0.15 or lower, the HT Iᵣ/I_{f} is a reversibility value of the hole transport material (HT) within an oxidation range at a scan rate of 100 mV/s, and the EB Iᵣ/I_{f} is a reversibility value of the electron blocking material (EB) within an oxidation range at a scan rate of 100 mV/s. The organic material layer includes a light emitting layer, the electron blocking layer is adjacent to the light emitting layer, and the hole transport layer is adjacent to a positive electrode. The electron blocking layer and the hole transport layer can be directly brought into contact with each other.

In an exemplary embodiment of the present specification, a value of (HT Iᵣ/I_{f})−(EB Iᵣ/I_{f}) is −0.17 or higher. In another exemplary embodiment, a value of (HT Iᵣ/I_{f})−(EB Iᵣ/I_{f}) is −0.12 or higher. In still another exemplary embodiment, a value of (HT Iᵣ/I_{f})−(EB Iᵣ/I_{f}) is −0.10 or higher. In yet another exemplary embodiment, a value of (HT Iᵣ/I_{f})−(EB is 0 or higher.

In an exemplary embodiment of the present specification, a value of (HT Iᵣ/I_{f})−(EB Iᵣ/I_{f}) is 0.1 or lower. In another exemplary embodiment, a value of (HT Iᵣ/I_{f})−(EB Iᵣ/I_{f}) is 0.1 or lower. In still another exemplary embodiment, a value of (HT Iᵣ/I_{f})−(EB Iᵣ/I_{f}) is 0.06 or lower.

In an exemplary embodiment of the present specification, the organic material layer includes a blue light emitting dopant material (BD), and the blue light emitting dopant material (BD) has a LUMO absolute value of 2.40 eV to 2.74 eV, and a reversibility value (Iᵣ/I_{f}) larger than [−23.14+8.458×(the LUMO absolute value)] within a reduction value at a scan rate of 100 mV/s at the time of measuring cyclic voltage current.

The LUMO absolute value of the blue light emitting dopant material (BD) is measured by AC3. In an exemplary embodiment, the LUMO absolute value of the blue light emitting dopant material (BD) is a work function value measured by an AC3 device.

In an exemplary embodiment of the present specification, the LUMO absolute value of the blue light emitting dopant material (BD) is 2.40 eV to 2.74 eV when measured by AC3. In one exemplary embodiment, the reversibility value (Iᵣ/I_{f}) within a reduction range at a scan rate of 100 mV/s is larger than [−23.14+8.458×(the AC3 LUMO absolute value)]. In this case, the stability of the blue light emitting dopant material (BD) is enhanced. Therefore, service life characteristics of the organic light emitting device are improved.

In an exemplary embodiment of the present specification, the blue light emitting dopant material (BD) is an arylamine compound, a pyrene compound, a fluorene compound, or a boron polycyclic compound.

In an exemplary embodiment of the present specification, the blue light emitting dopant material (BD) is a compound of any one of the following Formulae 3 to 6:

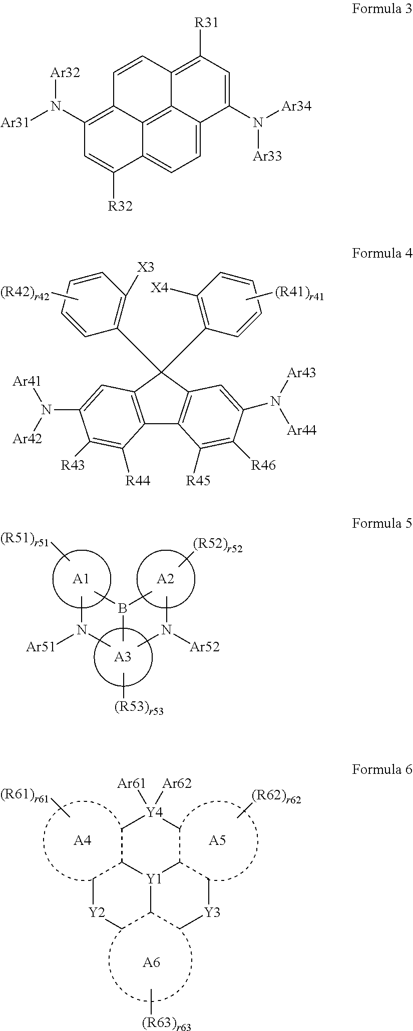

wherein in Formulae 3 to 6:

R31 and R32 are the same as or different from each other, and are each independently hydrogen, deuterium, a substituted or unsubstituted alkyl group, a substituted or unsubstituted silyl group, or a substituted or unsubstituted aryl group;

X3 and X4 are each hydrogen or deuterium, or are directly single-bonded to each other to form a ring;

R41 and R42 are the same as or different from each other, and are each independently hydrogen, deuterium, or a substituted or unsubstituted alkyl group;

R43 to R46 are the same as or different from each other, and are each independently hydrogen, deuterium, a substituted or unsubstituted alkyl group, or a substituted or unsubstituted aryl group, or adjacent substituents are bonded to each other to form a substituted or unsubstituted ring;

Ar31 to Ar34 and Ar41 to Ar44 are the same as or different from each other, and are each independently a substituted or unsubstituted aryl group or substituted or unsubstituted heteroaryl group;

A1 to A6 are the same as or different from each other, and are each independently a monocyclic to polycyclic aromatic hydrocarbon ring or monocyclic to polycyclic aromatic hetero ring;

R51 to R53 and R61 to R63 are the same as or different from each other, and are each independently hydrogen, deuterium, a substituted or unsubstituted alkyl group, a substituted or unsubstituted silyl group, a substituted or unsubstituted amine group, a substituted or unsubstituted aryl group, or a substituted or unsubstituted heteroaryl group, or are bonded to an adjacent substituent to form a substituted or unsubstituted aromatic ring or a substituted or unsubstituted aliphatic ring;

Y1 is B or N;

Y2 is O, S, or N(Ar63) (Ar64);

Y3 is O, S, or N(Ar65) (Ar66);

Y4 is C or Si;

Ar51, Ar52, and Ar61 to Ar66 are the same as or different from each other, and are each independently a substituted or unsubstituted alkyl group, a substituted or unsubstituted aryl group, or a substituted or unsubstituted heteroaryl group, or are bonded to an adjacent substituent to form a substituted or unsubstituted aromatic ring or a substituted or unsubstituted aliphatic ring; and

r41, r42, r51 to r53, and r61 to r63 are each an integer from 0 to 4, and when r41, r42, r51 to r53, and r61 to r63 are 2 or higher, substituents in the parenthesis are the same as or different from each other.

In an exemplary embodiment of the present specification, R31 and R32 are the same as or different from each other, and are each independently hydrogen, deuterium, an alkyl group having 1 to 6 carbon atoms, an alkylsilyl group having 1 to 10 carbon atoms, an arylsilyl group having 6 to 50 carbon atoms, or an aryl group having 6 to 30 carbon atoms.

In an exemplary embodiment of the present specification, R31 and R32 are the same as or different from each other, and are each independently hydrogen, deuterium, a methyl group, an ethyl group, a propyl group, an isopropyl group, a t-butyl group, a trimethylsilyl group, a triphenylsilyl group, a phenyl group, a biphenyl group, or a naphthyl group.

In an exemplary embodiment of the present specification, Ar31 to Ar34 are the same as or different from each other, and are each independently a substituted or unsubstituted aryl group having 6 to 30 carbon atoms, or a substituted or unsubstituted heteroaryl group having 2 to 30 carbon atoms.

In an exemplary embodiment of the present specification, Ar31 to Ar34 are the same as or different from each other, and are each independently a substituted or unsubstituted phenyl group, a naphthyl group, a dibenzofuran group, or a dibenzothiophene group.

In an exemplary embodiment of the present specification, when X3 and X4 are directly single-bonded to each other to form a ring, the core of Formula 4 includes spirobifluorene.

In an exemplary embodiment of the present specification, R41 and R42 are the same as or different from each other, and are each independently hydrogen, deuterium, or a substituted or unsubstituted alkyl group having 1 to 6 carbon atoms.

In an exemplary embodiment of the present specification, R43 to R46 are the same as or different from each other, and are each independently hydrogen, deuterium, a substituted or unsubstituted alkyl group having 1 to 6 carbon atoms, or a substituted or unsubstituted aryl group having 6 to 30 carbon atoms, or adjacent substituents are bonded to each other to form a pentagonal hetero ring in which a substituted or unsubstituted aromatic ring is fused.

In an exemplary embodiment of the present specification, R43 to R46 are the same as or different from each other, and are each independently hydrogen, or one or more substituents selected from the group consisting of deuterium, an alkyl group having 1 to 6 carbon atoms, and an aryl group having 6 to 30 carbon atoms, or a substituent to which two or more substituents selected from the group are linked.

In an exemplary embodiment of the present specification, R43 and R44 are bonded to each other to form a substituted or unsubstituted benzofuran ring or a substituted or unsubstituted benzothiophene ring.

In an exemplary embodiment of the present specification, R45 and R46 are bonded to each other to form a substituted or unsubstituted benzofuran ring or a substituted or unsubstituted benzothiophene ring.

In an exemplary embodiment of the present specification, Ar41 to Ar44 are the same as or different from each other, and are each independently a substituted or unsubstituted aryl group having 6 to 30 carbon atoms, or a substituted or unsubstituted heteroaryl group having 2 to 30 carbon atoms.

In an exemplary embodiment of the present specification, Ar41 to Ar44 are the same as or different from each other, and are each independently a phenyl group which is unsubstituted or substituted with a tert-butyl group; a naphthyl group; a dibenzofuran group; or a dibenzothiophene group.

In an exemplary embodiment of the present specification, A1 to A6 are the same as or different from each other, and are each independently a monocyclic to polycyclic aromatic hydrocarbon ring, or a monocyclic to polycyclic aromatic hetero ring.

In an exemplary embodiment of the present specification, A1 to A6 are the same as or different from each other, and are each independently a monocyclic to bicyclic aromatic hydrocarbon ring, or a monocyclic to bicyclic aromatic hetero ring containing O or S.

In an exemplary embodiment of the present specification, A1 to A6 are the same as or different from each other, and are each independently a benzene ring or a thiophene ring.

In an exemplary embodiment of the present specification, A1 to A6 are each a benzene ring.

In an exemplary embodiment of the present specification, R51 to R53 and R61 to R63 are the same as or different from each other, and are each independently hydrogen, or one or more substituents selected from the group consisting of deuterium, an alkyl group having 1 to 6 carbon atoms, an alkylsilyl group having 1 to 30 carbon atoms, an arylsilyl group having 6 to 50 carbon atoms, an alkylamine group having 1 to 30 carbon atoms, an alkylarylamine group having 1 to 50 carbon atoms, an arylamine group having 6 to 50 carbon atoms, an aryl group having 6 to 30 carbon atoms, and a heteroaryl group having 2 to 30 carbon atoms, or a substituent to which two or more substituents selected from the group are linked, or adjacent substituents are bonded to each other to form an aliphatic hydrocarbon ring having 3 to 60 carbon atoms, which is unsubstituted or substituted with the substituent.

In an exemplary embodiment of the present specification, R53 and R63 are the same as or different from each other, and are each independently a substituted or unsubstituted alkylamine group having 1 to 30 carbon atoms, a substituted or unsubstituted alkylarylamine group having 1 to 50 carbon atoms, or a substituted or unsubstituted arylamine group having 6 to 50 carbon atoms.

In an exemplary embodiment of the present specification, Ar51, Ar52, and Ar61 to Ar66 are the same as or different from each other, and are each independently a substituted or unsubstituted alkyl group having 1 to 10 carbon atoms, a substituted or unsubstituted aryl group having 6 to 30 carbon atoms, or a substituted or unsubstituted heteroaryl group having 2 to 30 carbon atoms.

In an exemplary embodiment of the present specification, Ar51, Ar52, and Ar61 to Ar66 are the same as or different from each other, and are each independently an alkyl group having 1 to 10 carbon atoms, which is unsubstituted or substituted with an aryl group; an aryl group having 6 to 30 carbon atoms, which is unsubstituted or substituted with an aryl group; or a heteroaryl group having 2 to 30 carbon atoms.

In an exemplary embodiment of the present specification, Ar51, Ar52, and Ar61 to Ar66 are the same as or different from each other, and are each independently a phenyl group, a biphenyl group, a naphthyl group, a dimethylfluorenyl group, a diphenylfluorenyl group, a dibenzofuran group, or a dibenzothiophene group.

In an exemplary embodiment of the present specification, R51 and Ar51 are bonded to each other to form a substituted or unsubstituted aromatic ring, or a substituted or unsubstituted aliphatic ring.

In an exemplary embodiment of the present specification, R52 and Ar52 are bonded to each other to form a substituted or unsubstituted aromatic ring, or a substituted or unsubstituted aliphatic ring.

In an exemplary embodiment of the present specification, R51 and Ar51 are bonded to each other to form a substituted or unsubstituted monocyclic to polycyclic aromatic hydrocarbon ring, or a substituted or unsubstituted monocyclic to polycyclic aliphatic hydrocarbon ring.

In an exemplary embodiment of the present specification, R52 and Ar52 are bonded to each other to form a substituted or unsubstituted monocyclic to polycyclic aromatic hydrocarbon ring, or a substituted or unsubstituted monocyclic to polycyclic aliphatic hydrocarbon ring.

In an exemplary embodiment of the present specification, R51 and Ar51 are bonded to each other to form a substituted or unsubstituted benzene ring, a substituted or unsubstituted cyclohexane ring, or a substituted or unsubstituted cyclopentane ring.

In an exemplary embodiment of the present specification, R52 and Ar52 are bonded to each other to form a substituted or unsubstituted benzene ring, a substituted or unsubstituted cyclohexane ring, or a substituted or unsubstituted cyclopentane ring.

In an exemplary embodiment of the present specification, the blue light emitting dopant material (BD) is selected from among the following compounds:

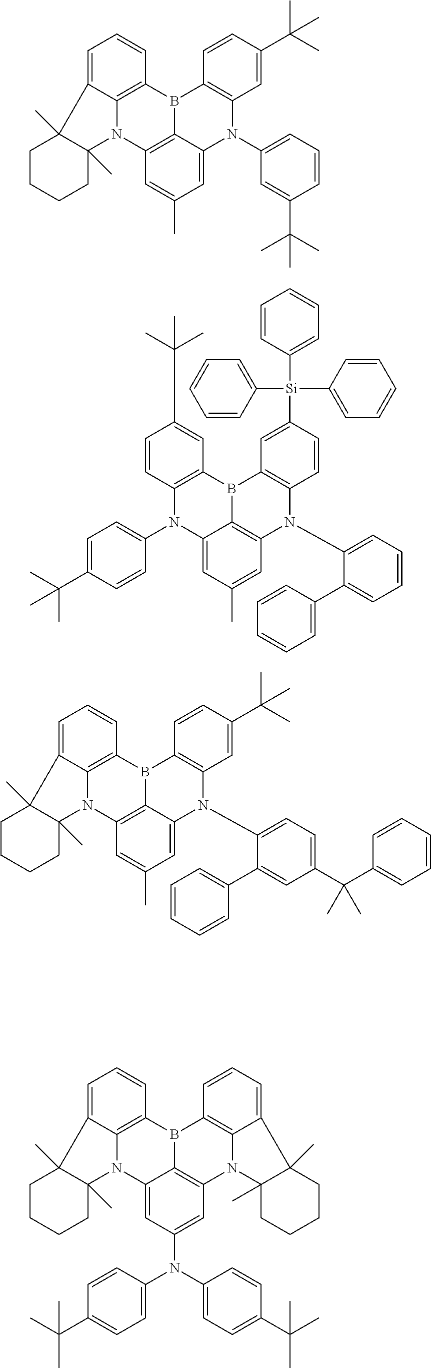

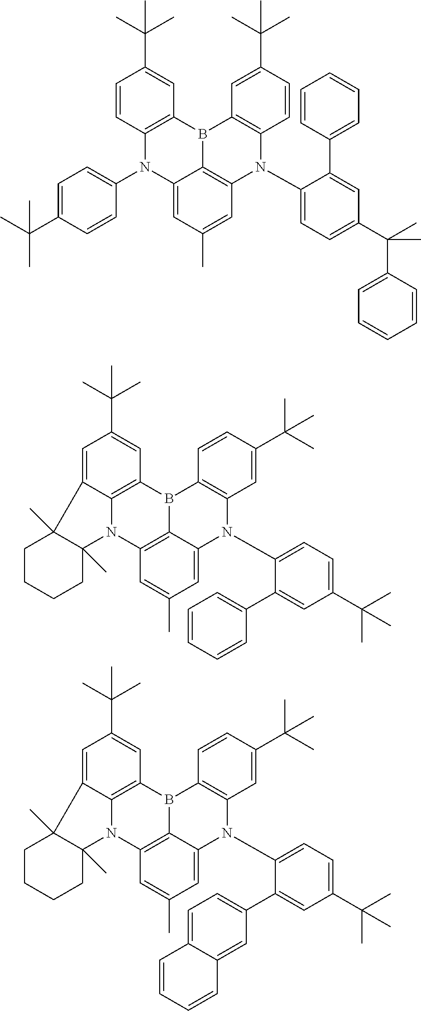 [SMILES: Cc1cc2c3c(c1)N(c1ccc(C(C)(C)c4ccccc4)cc1-c1ccccc1)c1ccc(C(C)(C)C)cc1B3c1cc(C(C)(C)C)ccc1N2c1ccc(C(C)(C)C)cc1 ; Cc1cc2c3c(c1)N1c4c(cc(C(C)(C)C)cc4C4(C)CCCCC14C)B3c1ccc(C(C)(C)C)cc1N2c1ccc(C(C)(C)C)cc1-c1ccccc1 ; Cc1cc2c3c(c1)N1c4c(cc(C(C)(C)C)cc4C4(C)CCCCC14C)B3c1ccc(C(C)(C)C)cc1N2c1ccc(C(C)(C)C)cc1-c1ccc2ccccc2c1]

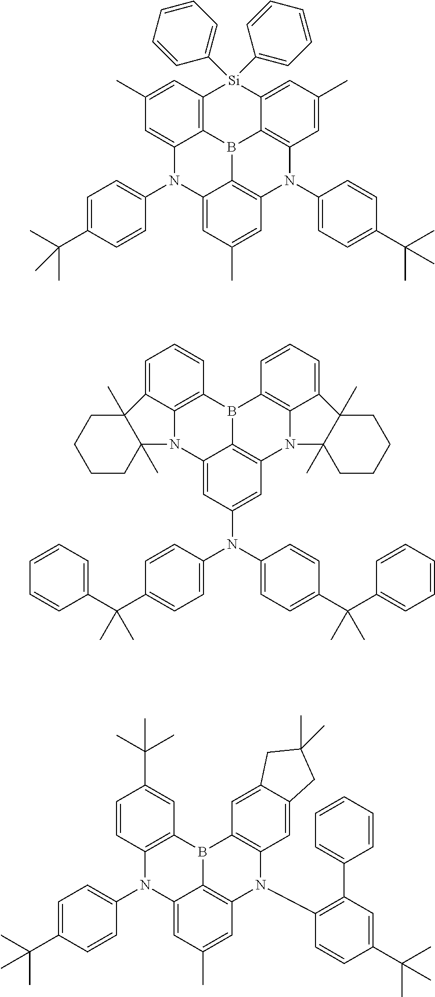

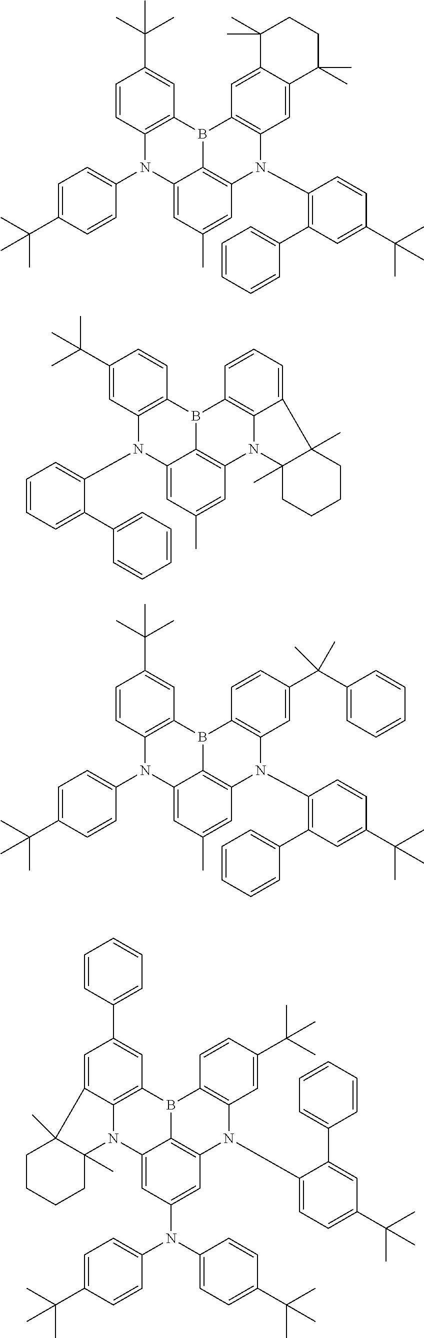

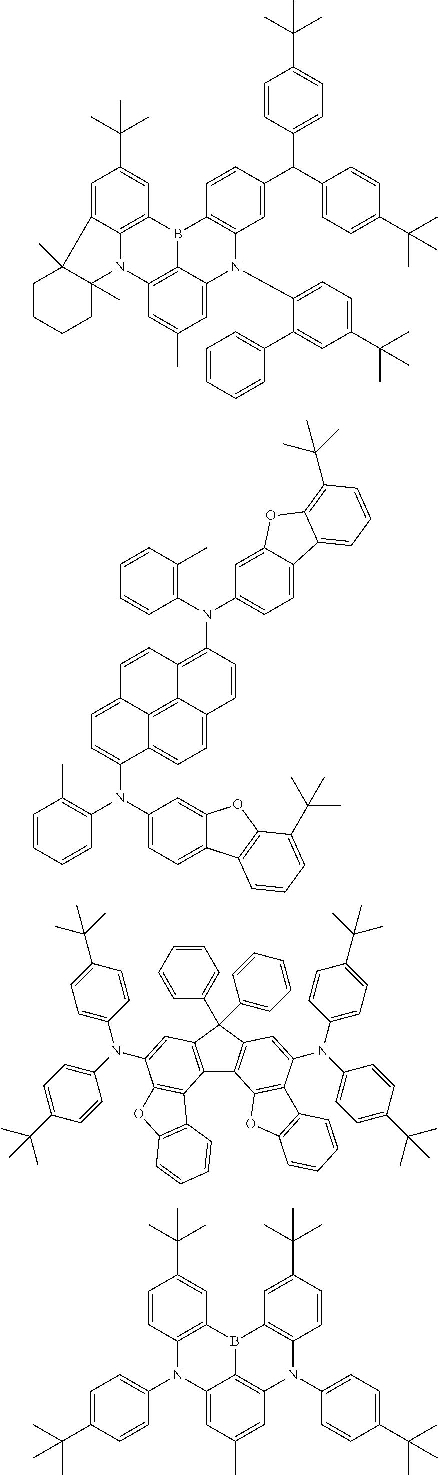

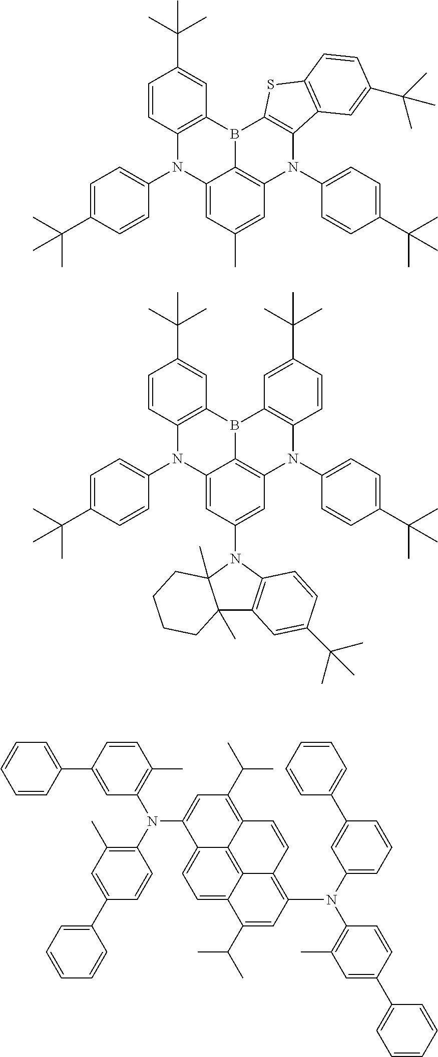

In an exemplary embodiment of the present specification, the organic material layer includes a blue light emitting host material (BH), and the blue light emitting host material (BH) has a reversibility value (Iᵣ/I_{f}) of [1.34×(the dipole moment)−0.293] or higher within an oxidation range at a scan rate of 500 mV/s and a reversibility value (Iᵣ/I_{f}) of 0.95 or higher within a reduction range at a scan rate of 10 mV/s, at the time of measuring cyclic voltage current.

In an exemplary embodiment of the present specification, the blue light emitting host material (BH) has a reversibility value (Iᵣ/I_{f}) of 0.95 or higher, preferably 0.96 or higher, and more preferably 0.97 or higher within a reduction range at a scan rate of 10 mV/s at the time of measuring cyclic voltage current.

In an exemplary embodiment of the present specification, the blue light emitting host material (BH) has a reversibility value (Iᵣ/I_{f}) of 1.2 or lower, preferably 1.1 or lower within a reduction range at a scan rate of 10 mV/s at the time of measuring cyclic voltage current.

In an exemplary embodiment of the present specification, the blue light emitting host material (BH) is a compound of the following Formula H. Specifically, the blue light emitting host material (BH) is used in the same organic material layer as the blue light emitting dopant material.

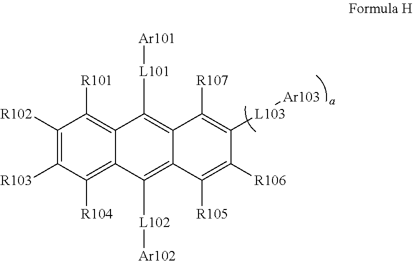

wherein in Formula H:

L101 to L103 are the same as or different from each other, and are each independently a direct bond, a substituted or unsubstituted arylene group, or a substituted or unsubstituted heteroarylene group;

R101 to R107 are the same as or different from each other, and are each independently hydrogen, deuterium, a substituted or unsubstituted alkyl group, a substituted or unsubstituted cycloalkyl group, a substituted or unsubstituted silyl group, a substituted or unsubstituted aryl group, or a substituted or unsubstituted heteroaryl group:

Ar101 to Ar103 are the same as or different from each other, and are each independently a substituted or unsubstituted aryl group or a substituted or unsubstituted heteroaryl group; and

a is 0 or 1.

In an exemplary embodiment of the present specification, when a is 0, hydrogen or deuterium is linked to the position of -L103-Ar103.

In an exemplary embodiment of the present specification, L101 to L103 are the same as or different from each other, and are each independently a direct bond, a substituted or unsubstituted arylene group having 6 to 30 carbon atoms, or a substituted or unsubstituted heteroarylene group having 2 to 30 carbon atoms and including N, O, or S.

In an exemplary embodiment of the present specification, L101 to L103 are the same as or different from each other, and are each independently a direct bond, a substituted or unsubstituted phenylene group, a substituted or unsubstituted biphenylene group, a substituted or unsubstituted naphthylene group, a substituted or unsubstituted phenanthrenylene group, a substituted or unsubstituted divalent dibenzofuran group, or a substituted or unsubstituted divalent dibenzothiophene group.

In an exemplary embodiment of the present specification, L101 to L103 are the same as or different from each other, and are each independently a direct bond, a phenylene group, a biphenylene group, a naphthylene group, or a phenanthrenylene group.

In an exemplary embodiment of the present specification, Ar101 to Ar103 are the same as or different from each other, and are each independently a substituted or unsubstituted aryl group having 6 to 30 carbon atoms, or a substituted or unsubstituted heteroaryl group having 2 to 30 carbon atoms.

In an exemplary embodiment of the present specification, Ar101 to Ar103 are the same as or different from each other, and are each independently a substituted or unsubstituted phenyl group, a substituted or unsubstituted biphenyl group, a substituted or unsubstituted terphenyl group, a substituted or unsubstituted naphthyl group, a substituted or unsubstituted anthracene group, a substituted or unsubstituted phenanthryl group, a substituted or unsubstituted phenalene group, a substituted or unsubstituted fluorenyl group, a substituted or unsubstituted benzofluorenyl group, a substituted or unsubstituted furan group, a substituted or unsubstituted thiophene group, a substituted or unsubstituted dibenzofuran group, a substituted or unsubstituted naphthobenzofuran group, a substituted or unsubstituted dibenzothiophene group, or a substituted or unsubstituted naphthobenzothiophene group.

In an exemplary embodiment of the present specification, Ar101 to Ar103 are the same as or different from each other, and are each independently a phenyl group, a biphenyl group, a naphthyl group, a phenanthryl group, a dibenzofuran group, or a dibenzothiophene group.

In an exemplary embodiment of the present specification, R101 to R107 are hydrogen or deuterium.

In an exemplary embodiment of the present specification, Formula H is any one compound selected from among the following compounds:

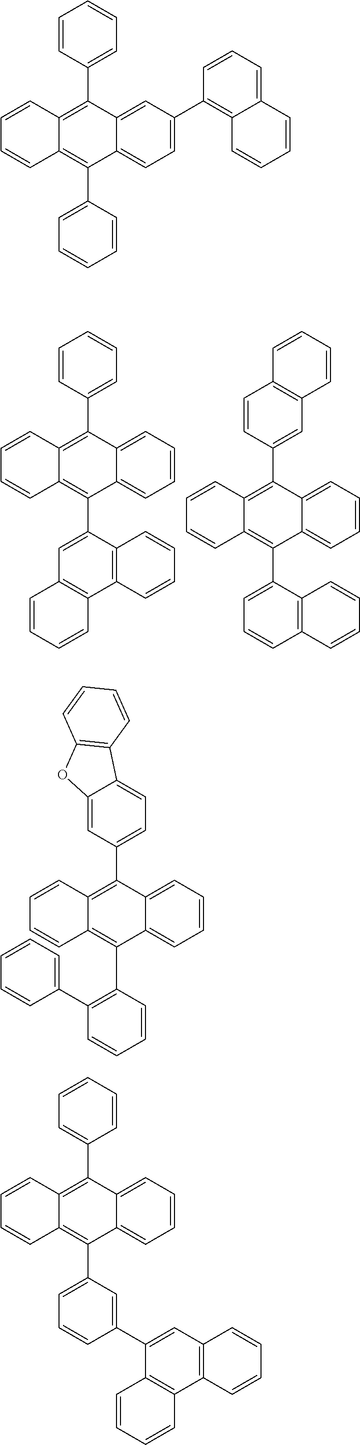

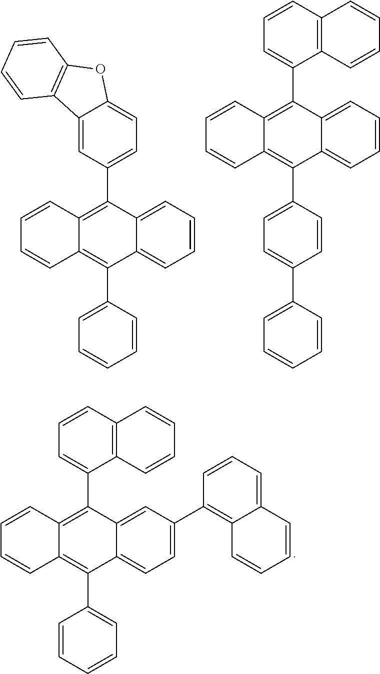

In an exemplary embodiment of the present specification, the organic material layer including the blue light emitting dopant material (BD) further includes a blue light emitting host material (BH) and has a [(the LUMO absolute value of the blue light emitting host material (BH))−(the LUMO absolute value of the blue light emitting dopant material (BD))] value of 0.16 eV to 0.75 eV.

The LUMO absolute value of the blue light emitting host material (BH) is measured by AC3. In an exemplary embodiment, the LUMO absolute value of the blue light emitting host material (BH) is a work function value measured by an AC3 device.

In an exemplary embodiment of the present specification, [the LUMO absolute value of the blue light emitting host material (BH)]−[the LUMO absolute value of the blue light emitting dopant material (BD)] is 0.18 eV or higher, preferably 0.20 eV or higher.

In an exemplary embodiment of the present specification, [the LUMO absolute value of the blue light emitting host material (BH)]−[the LUMO absolute value of the blue light emitting dopant material (BD)] is 0.65 eV or lower, preferably 0.60 eV or lower.

The organic material layer according to an exemplary embodiment of the present specification includes a blue light emitting layer, and the blue light emitting layer includes the compound of any one of Formulae 3 to 6 as a dopant of the light emitting layer, and includes the compound of Formula H as a host of the light emitting layer.

In an exemplary embodiment of the present specification, the content of the compound of any one of Formulae 3 to 6 is 0.01 part by weight to 30 parts by weight; 0.1 part by weight to 20 parts by weight; or 0.5 part by weight to 10 parts by weight, based on 100 parts by weight of the compound of Formula H.

In an exemplary embodiment of the present specification, the organic material layer includes an electron transport material (ET), and the electron transport material (ET) has a LUMO absolute value of 2.60 eV to 2.90 eV, and a reversibility value (Iᵣ/I_{f}) larger than [4.96−1.535×(the LUMO absolute value)] within a reduction range at a scan rate of 100 mV/s at the time of measuring cyclic voltage current.

The LUMO absolute value of the electron transport material (ET) is measured by AC3. In an exemplary embodiment, the LUMO absolute value of the electron transport material (ET) is a work function value measured by an AC3 device.

In an exemplary embodiment of the present specification, the electron transport material (ET) is a triazine-based or pyrimidine-based compound.

In an exemplary embodiment of the present specification, the electron transport material (ET) is of the following Formula 8:

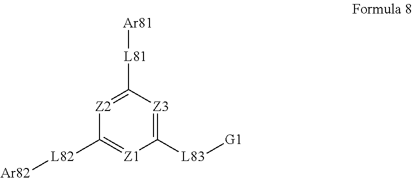

wherein in Formula 8:

at least one of Z1 to Z3 is N, and the others are CH;

L81 to L83 are the same as or different from each other, and are each independently a direct bond, a substituted or unsubstituted arylene group, or a substituted or unsubstituted heteroarylene group;

Ar81 and Ar82 are the same as or different from each other, and are each independently a substituted or unsubstituted aryl group or a substituted or unsubstituted heteroaryl group;

G1 is a monovalent substituent of any one of the following Formulae 801 to 804:

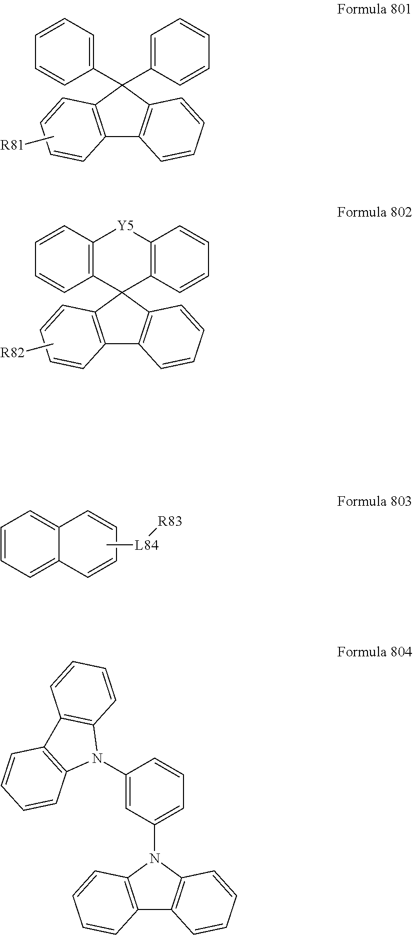

wherein in Formulae 801 to 804:

any one carbon is linked to L83 of Formula 8;

Y5 is O or S;

L84 is a substituted or unsubstituted arylene group; and

R81 to R83 are the same as or different from each other, and are each independently hydrogen, deuterium, a cyano group, an aryl group, or an aryl group which is substituted with a cyano group.

In an exemplary embodiment of the present specification, the electron transport material (ET) is of the following Formula 12:

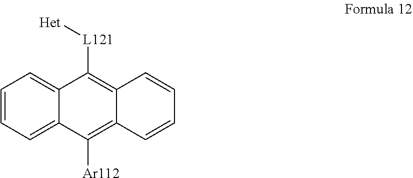

wherein in Formula 12:

Het is a substituted or unsubstituted N-containing heteroaryl group;

Ar112 is a substituted or unsubstituted aryl group or a substituted or unsubstituted aryl group; and

L121 is a substituted or unsubstituted arylene group or a substituted or unsubstituted heteroarylene group.

In an exemplary embodiment of the present specification, Z1 to Z3 are all N.

In an exemplary embodiment of the present specification, Z1 and Z2 are N, and Z3 is CH.

In an exemplary embodiment of the present specification, L81 to L84 and L121 are the same as or different from each other, and are each independently a direct bond, a substituted or unsubstituted arylene group having 6 to 30 carbon atoms, or a substituted or unsubstituted heteroarylene group having 2 to 30 carbon atoms and including N, O, or S.

In an exemplary embodiment of the present specification, L81 to L84 and L121 are the same as or different from each other, and are each independently a direct bond, a phenylene group, or a naphthylene group.

In an exemplary embodiment of the present specification, L81 to L83 and L121 are the same as or different from each other, and are each independently a direct bond or a phenylene group.

In an exemplary embodiment of the present specification, L84 is a direct bond, a phenylene group, or a naphthylene group.

In an exemplary embodiment of the present specification, Ar81 and Ar82 are the same as or different from each other, and are each independently a substituted or unsubstituted aryl group having 6 to 30 carbon atoms, or a substituted or unsubstituted heteroaryl group having 2 to 30 carbon atoms.

In an exemplary embodiment of the present specification, Ar81 and Ar82 are the same as or different from each other, and are each independently a substituted or unsubstituted phenyl group, a substituted or unsubstituted biphenyl group, a substituted or unsubstituted terphenyl group, a substituted or unsubstituted naphthyl group, a substituted or unsubstituted triazine group, or a substituted or unsubstituted pyridine group.

In an exemplary embodiment of the present specification, G1 is any one structure selected from among the following structures:

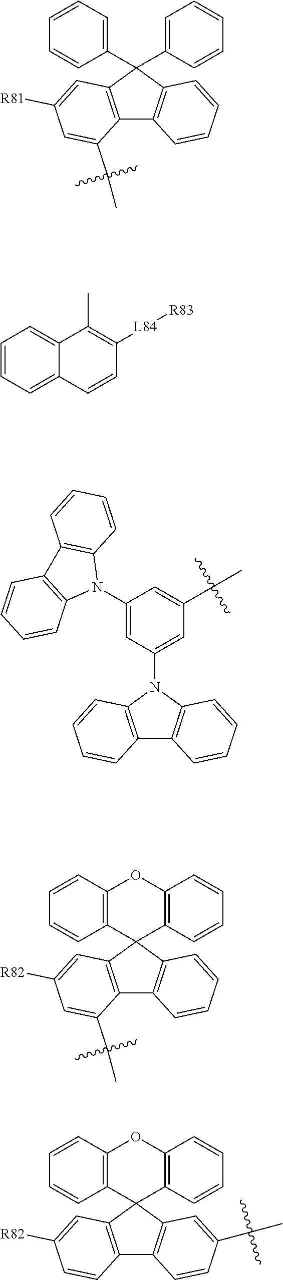

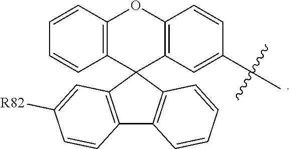

In the structures, the definitions of L84 and R81 to R83 are the same as those described above.

In an exemplary embodiment of the present specification, R81 to R83 are the same as or different from each other, and are each independently hydrogen, deuterium, a cyano group, an aryl group having 6 to 30 carbon atoms, or an aryl group having 6 to 30 carbon atoms, which is substituted with a cyano group.

In an exemplary embodiment of the present specification, R81 to R83 are the same as or different from each other, and are each independently hydrogen, deuterium, a cyano group, a phenyl group, or a phenyl group which is substituted with a cyano group.

In an exemplary embodiment of the present specification, Het is a substituted or unsubstituted N-containing heteroaryl group having 2 to 20 carbon atoms.

In an exemplary embodiment of the present specification, Het is an N-containing heteroaryl group having 2 to 20 carbon atoms, which is unsubstituted or substituted with an alkyl group having 1 to 6 carbon atoms.

In an exemplary embodiment of the present specification, Het is a benzimidazole group which is unsubstituted or substituted with an ethyl group.

In an exemplary embodiment of the present specification, the electron transport material (ET) is selected from among the following compounds:

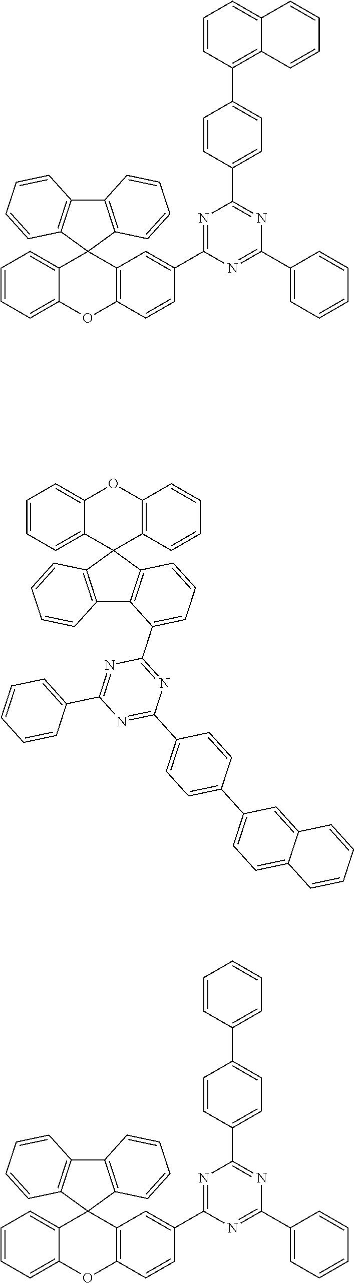 [SMILES: c1ccc(-c2nc(-c3ccc(-c4cccc5ccccc45)cc3)nc(-c3ccc4c(c3)C3(c5ccccc5O4)c4ccccc4-c4ccccc43)n2)cc1 ; c1ccc(-c2nc(-c3ccc(-c4ccc5ccccc5c4)cc3)nc(-c3cccc4c3-c3ccccc3C43c4ccccc4Oc4ccccc43)n2)cc1 ; c1ccc(-c2ccc(-c3nc(-c4ccccc4)nc(-c4ccc5c(c4)C4(c6ccccc6O5)c5ccccc5-c5ccccc54)n3)cc2)cc1]

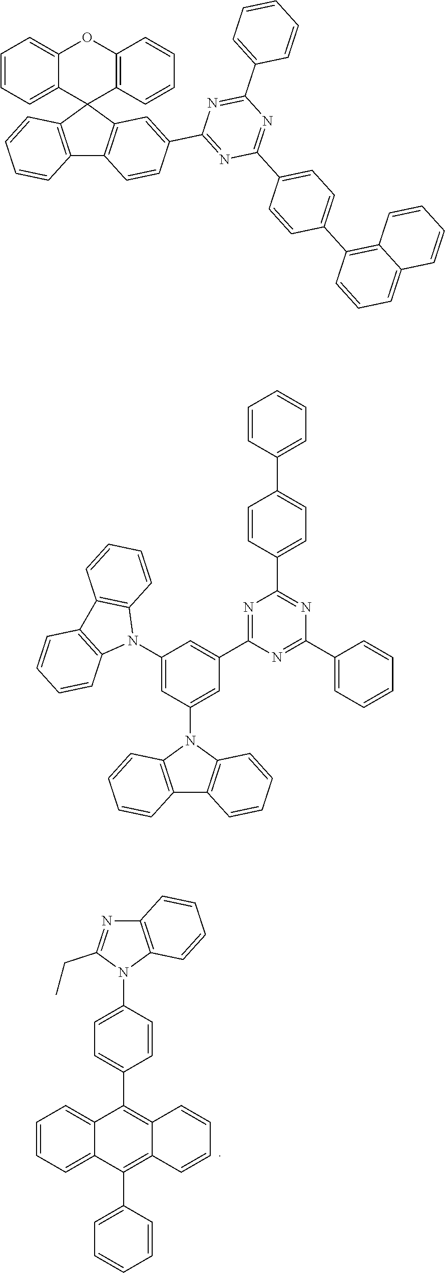 [SMILES: c1ccc(-c2nc(-c3ccc(-c4cccc5ccccc45)cc3)nc(-c3ccc4c(c3)C3(c5ccccc5Oc5ccccc53)c3ccccc3-4)n2)cc1 ; c1ccc(-c2ccc(-c3nc(-c4ccccc4)nc(-c4cc(-n5c6ccccc6c6ccccc65)cc(-n5c6ccccc6c6ccccc65)c4)n3)cc2)cc1 ; CCc1nc2ccccc2n1-c1ccc(-c2c3ccccc3c(-c3ccccc3)c3ccccc23)cc1]

In an exemplary embodiment of the present specification, the organic material layer includes a hole blocking material (HB), and the hole blocking material (HB) has both a forward peak and an inverse peak at a scan rate of 100 mV/s at the time of measuring cyclic voltage current within an oxidation range.

In an exemplary embodiment of the present specification, the hole blocking material (HB) is a triazine-based or pyrimidine-based compound.

In an exemplary embodiment of the present specification, the hole blocking material (HB) is of the following Formula 9:

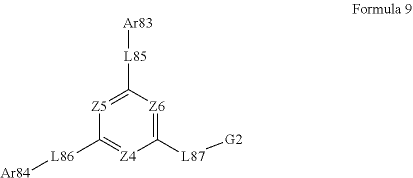

wherein in Formula 9:

at least one of Z4 to Z6 is N, and the others are CH;

L85 to L87 are the same as or different from each other, and are each independently a direct bond, a substituted or unsubstituted arylene group, or a substituted or unsubstituted heteroarylene group;

Ar83 and Ar84 are the same as or different from each other, and are each independently a substituted or unsubstituted aryl group or a substituted or unsubstituted heteroaryl group;

G2 is a monovalent substituent of the following Formula 901:

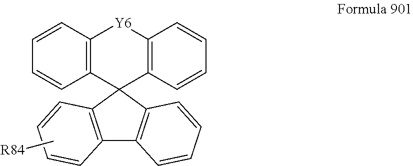

wherein in Formula 901:

any one carbon is linked to L87 of Formula 9;

Y6 is O or S; and

R84 is hydrogen, deuterium, a cyano group, an aryl group, or an aryl group which is substituted with a cyano group.

In an exemplary embodiment of the present specification, Z4 to Z6 are all N.

In an exemplary embodiment of the present specification, Z4 and Z5 are N, and Z6 is CH.

In an exemplary embodiment of the present specification, L85 to L87 are the same as or different from each other, and are each independently a direct bond, a substituted or unsubstituted arylene group having 6 to 30 carbon atoms, or a substituted or unsubstituted heteroarylene group having 2 to 30 carbon atoms and including N, O, or S.

In an exemplary embodiment of the present specification, L85 to L87 are the same as or different from each other, and are each independently a direct bond, a phenylene group, or a biphenylene group.

In an exemplary embodiment of the present specification, Ar83 and Ar84 are the same as or different from each other, and are each independently a substituted or unsubstituted aryl group having 6 to 30 carbon atoms, or a substituted or unsubstituted heteroaryl group having 2 to 30 carbon atoms.

In an exemplary embodiment of the present specification, Ar83 and Ar84 are the same as or different from each other, and each independently a substituted or unsubstituted phenyl group, a substituted or unsubstituted biphenyl group, a substituted or unsubstituted terphenyl group, a substituted or unsubstituted naphthyl group, or a substituted or unsubstituted pyridine group.

In an exemplary embodiment of the present specification, Ar83 and Ar84 are the same as or different from each other, and are each independently a phenyl group, a biphenyl group, a terphenyl group, or a naphthyl group.

In an exemplary embodiment of the present specification, G2 is any one structure selected from among the following structures:

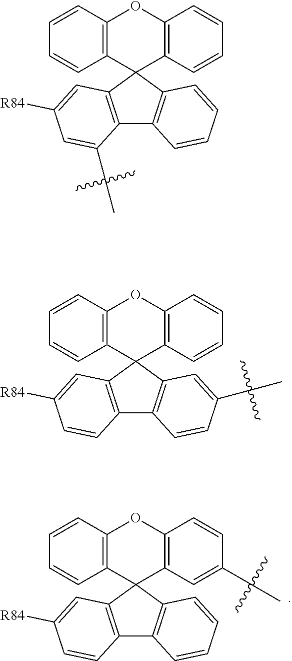

In the structures, the definition of R84 is the same as that described above.

In an exemplary embodiment of the present specification, R84 is hydrogen, deuterium, a cyano group, an aryl group having 6 to 30 carbon atoms, or an aryl group having 6 to 30 carbon atoms, which is substituted with a cyano group.

In an exemplary embodiment of the present specification, R84's are the same as or different from each other, and are each independently hydrogen, deuterium, a cyano group, a phenyl group, or a phenyl group which is substituted with a cyano group.

In an exemplary embodiment of the present specification, the hole blocking material (HB) is a compound of Formula 12.

In an exemplary embodiment of the present specification, the hole blocking material (HB) is of the following Formula 11:

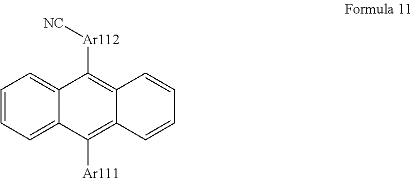

wherein in Formula 11:

Ar111 is a substituted or unsubstituted aryl group or a substituted or unsubstituted heteroaryl group; and

Ar112 is a substituted or unsubstituted arylene group or a substituted or unsubstituted heteroarylene group.

In an exemplary embodiment of the present specification, Ar111 is a substituted or unsubstituted aryl group having 6 to 30 carbon atoms.

In an exemplary embodiment of the present specification, Ar111 is a substituted or unsubstituted aryl group having 6 to 20 carbon atoms.

In an exemplary embodiment of the present specification, Ar111 is a phenyl group.

In an exemplary embodiment of the present specification, Ar112 is a substituted or unsubstituted arylene group having 6 to 30 carbon atoms.

In an exemplary embodiment of the present specification, Ar112 is an arylene group having 6 to 20 carbon atoms, which is unsubstituted or substituted with an alkyl group having 1 to 6 carbon atoms.

In an exemplary embodiment of the present specification, Ar112 is a dimethylfluorenylene group.

In an exemplary embodiment of the present specification, the hole blocking material (HB) is selected from among the following compounds:

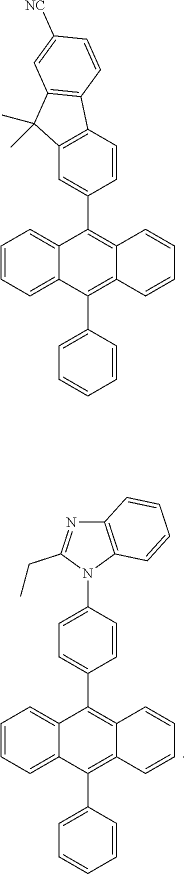 [SMILES: CC1(C)c2cc(C#N)ccc2-c2ccc(-c3c4ccccc4c(-c4ccccc4)c4ccccc34)cc21 ; CCc1nc2ccccc2n1-c1ccc(-c2c3ccccc3c(-c3ccccc3)c3ccccc23)cc1]

The present specification provides an organic light emitting device including the electron transport material (ET) and the hole blocking material (HB) described above. Specifically, the organic light emitting device includes an organic material layer, and the organic material layer includes an electron transport layer and a hole blocking layer. The electron transport layer includes the above-described electron transport material (ET), and the hole blocking layer includes the above-described hole blocking material (HB). In this case, [the LUMO absolute value of the electron transport material (ET)−the LUMO absolute value of the hole blocking material (HB)] is 0.05 eV to 0.3 eV. The organic material layer includes a light emitting layer, the hole blocking layer is adjacent to the light emitting layer, and the electron transport layer is adjacent to a negative electrode. The hole blocking layer and the electron transport layer can be directly brought into contact with each other.

The LUMO absolute value of the electron transport material (ET) and the LUMO absolute value of the hole blocking material (HB) are values measured by AC3. Specifically, the LUMO absolute value of the electron transport material (ET) and the LUMO absolute value of the hole blocking material (HB) are work function values measured by an AC3 device.

In an exemplary embodiment of the present specification, the organic material layer includes a light emitting host material (EML), and the light emitting host material (EML) has a reversibility value (Iᵣ/I_{f}) of [0.955−0.1786×(a reversibility value (Iᵣ/I_{f}) within an oxidation range)] or higher within a reduction range at a scan rate of 10 mV/s at the time of measuring cyclic voltage current.

In an exemplary embodiment of the present specification, the light emitting host material (EML) is a compound including triazine and indolocarbazole.

In an exemplary embodiment of the present specification, the light emitting host material (EML) is of the following Formula 10:

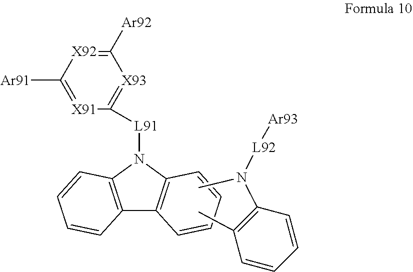

wherein in Formula 10:

at least one of X91 to X93 is N, and the others are CH;

L91 and L92 are the same as or different from each other, and are each independently a direct bond or a substituted or unsubstituted arylene group; and

Ar91 to Ar93 are the same as or different from each other, and are each independently a substituted or unsubstituted aryl group or a substituted or unsubstituted heteroaryl group.

In an exemplary embodiment of the present specification, X91 to X93 are all N.

In an exemplary embodiment of the present specification, X91 and X92 are N, and X93 is CH.

In an exemplary embodiment of the present specification, L91 and L92 are the same as or different from each other, and are each independently a direct bond; a substituted or unsubstituted arylene group having 6 to 30 carbon atoms, or a substituted or unsubstituted heteroarylene group having 2 to 30 carbon atoms and including N, O, or S.

In an exemplary embodiment of the present specification, L91 and L92 are the same as or different from each other, and are each independently a direct bond or an arylene group having 6 to 30 carbon atoms, which is unsubstituted or substituted with a cyano group.

In an exemplary embodiment of the present specification, L91 and L92 are the same as or different from each other, and are each independently a direct bond, a phenylene group which is unsubstituted or substituted with a cyano group, or a naphthyl group which is unsubstituted or substituted with a cyano group.

In an exemplary embodiment of the present specification, Ar91 to Ar93 are the same as or different from each other, and are each independently a substituted or unsubstituted aryl group having 6 to 30 carbon atoms, or a substituted or unsubstituted heteroaryl group having 2 to 30 carbon atoms.

In an exemplary embodiment of the present specification, Ar91 to Ar93 are the same as or different from each other, and are each independently a substituted or unsubstituted phenyl group, a substituted or unsubstituted biphenyl group, a substituted or unsubstituted terphenyl group, a substituted or unsubstituted dibenzofuran group, a substituted or unsubstituted dibenzothiophene group, a substituted or unsubstituted naphthyl group, a substituted or unsubstituted triazine group, or a substituted or unsubstituted pyridine group.

In an exemplary embodiment of the present specification, Ar91 to Ar93 are the same s or different from each other, are each independently a phenyl group, a biphenyl group, a terphenyl group, a naphthyl group, a dibenzofuran group, or a dibenzothiophene group, and are unsubstituted or substituted with deuterium.

In an exemplary embodiment of the present specification, Formula 10 is of any one of the following Formulae 10-1 to 10-7:

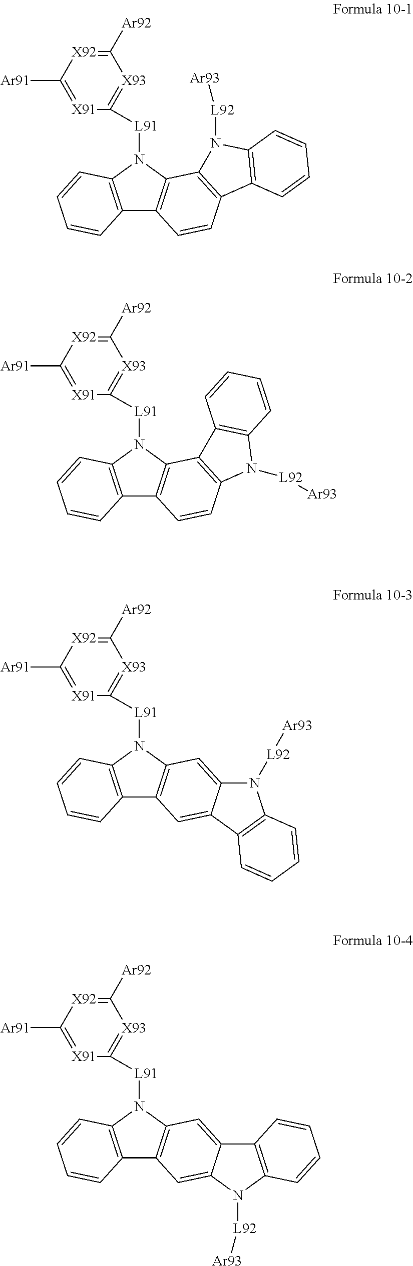

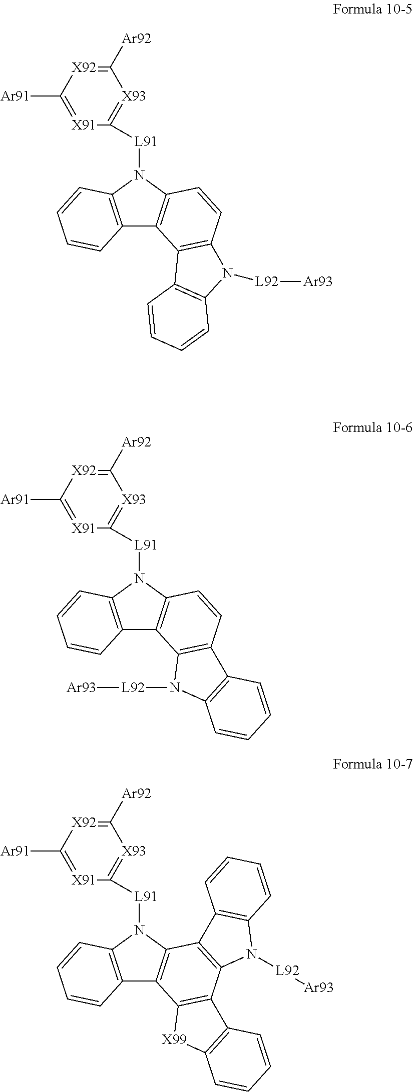

wherein in Formulae 10-1 to 10-7, the definitions of X91 to X93, Ar91 to Ar93, L91, and L92 are the same as those defined in Formula 10, and X99 is O or S.

In an exemplary embodiment of the present specification, the light emitting host material (EML) is selected from the following compounds:

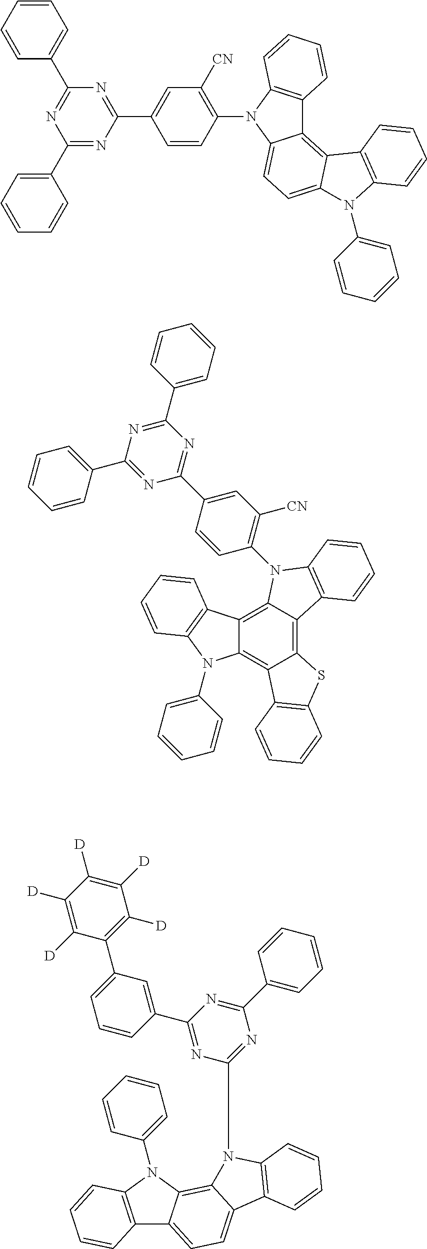 [SMILES: N#Cc1cc(-c2nc(-c3ccccc3)nc(-c3ccccc3)n2)ccc1-n1c2ccccc2c2c3c4ccccc4n(-c4ccccc4)c3ccc21 ; N#Cc1cc(-c2nc(-c3ccccc3)nc(-c3ccccc3)n2)ccc1-n1c2ccccc2c2c3sc4ccccc4c3c3c(c4ccccc4n3-c3ccccc3)c21 ; [2H]c1c([2H])c([2H])c(-c2cccc(-c3nc(-c4ccccc4)nc(-n4c5ccccc5c5ccc6c7ccccc7n(-c7ccccc7)c6c54)n3)c2)c([2H])c1[2H]]

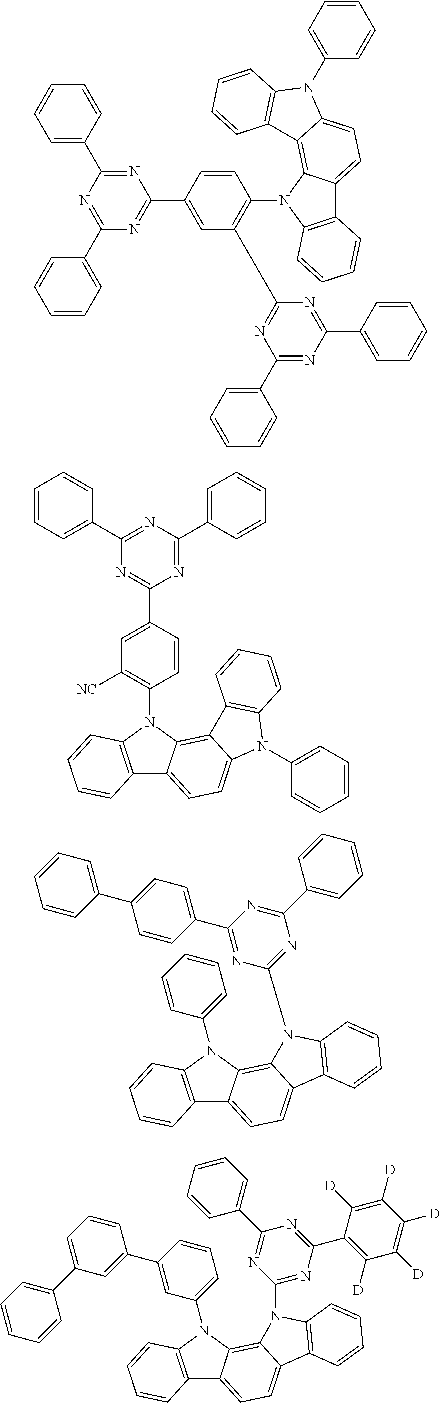

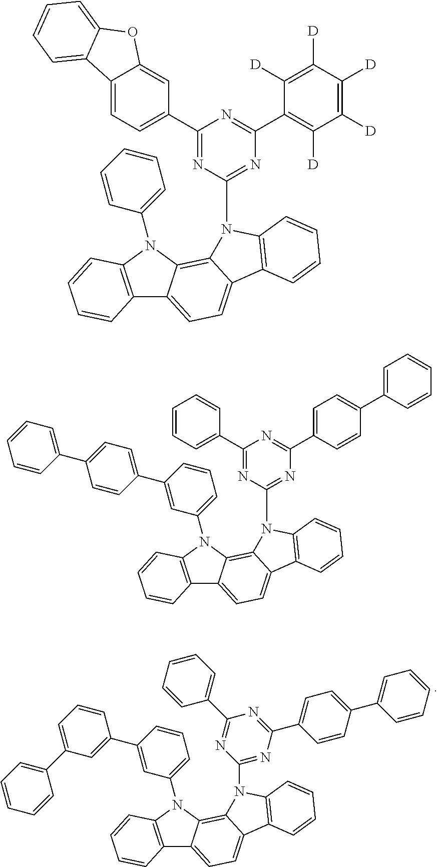 [SMILES: [2H]c1c([2H])c([2H])c(-c2nc(-c3ccc4c(c3)oc3ccccc34)nc(-n3c4ccccc4c4ccc5c6ccccc6n(-c6ccccc6)c5c43)n2)c([2H])c1[2H] ; c1ccc(-c2ccc(-c3cccc(-n4c5ccccc5c5ccc6c7ccccc7n(-c7nc(-c8ccccc8)nc(-c8ccc(-c9ccccc9)cc8)n7)c6c54)c3)cc2)cc1 ; c1ccc(-c2ccc(-c3nc(-c4ccccc4)nc(-n4c5ccccc5c5ccc6c7ccccc7n(-c7cccc(-c8cccc(-c9ccccc9)c8)c7)c6c54)n3)cc2)cc1]

In an exemplary embodiment of the present specification, the organic material layer including the light emitting host material (EML) is a light emitting layer. The light emitting region of the light emitting layer is green. That is, the light emitting layer including the light emitting host material (EML) has a maximum light emitting peak of 495 nm to 570 nm.

In another exemplary embodiment, the organic material layer including the hole transport material (HT) further includes an electron blocking material (EB) and has a [(HT Iᵣ/I_{f})−(EB Iᵣ/I_{f})] value of 0.15 or less, the HT Iᵣ/I_{f} is a reversibility value of the hole transport material (HT) within an oxidation range at a scan rate of 100 mV/s, and the EB Iᵣ/I_{f} is a reversibility value of the electron blocking material (EB) within an oxidation range at a scan rate of 100 mV/s. In this case, the hole transport material (HT) and the electron blocking material (EB) are each included in different organic material layers, an organic material layer including the electron blocking material (EB) is adjacent to the light emitting layer, and an organic material layer including the hole transport material (HT) is adjacent to a positive electrode. In an exemplary embodiment, the organic material layer including the electron blocking material (EB) and the organic material layer including the hole transport material (HT) are directly brought into contact with each other.

In still another exemplary embodiment, the organic material layer including the blue light emitting dopant material (BD) further includes a blue light emitting host material (BH) and has a [(the LUMO absolute value of the blue light emitting host material (BH))−(the LUMO absolute value of the blue light emitting dopant material (BD))] value of 0.16 eV to 0.75 eV. In this case, the blue light emitting dopant material (BD) and the blue light emitting host material (BH) are included in the same layer.

In yet another exemplary embodiment, the organic material layer including the electron transport material (ET) further includes a hole blocking material (HB) and has a [the LUMO absolute value of the electron transport material (ET)−the LUMO absolute value of the hole blocking material (HB)] of 0.05 eV to 0.3 eV. In this case, the electron transport material (ET) and the hole blocking material (HB) are each included in different organic material layers, an organic material layer including the hole blocking material (HB) is adjacent to the light emitting layer, and an organic material layer including the electron transport material (ET) is adjacent to a negative electrode. In an exemplary embodiment, the organic material layer including the hole blocking material (HB) and the organic material layer including the electron transport material (ET) are directly brought into contact with each other.

In another exemplary embodiment, the organic material layer including the light emitting host material (EML) further includes an electron transport material (ET) and has a [(the LUMO absolute value of the light emitting host material (EML))−(the LUMO absolute value of the electron transport material (ET))] value of 0.15 eV to 0.35 eV. In this case, the light emitting host material (EML) and the electron transport material (ET) are each included in different organic material layers, an organic material layer including the light emitting host material (EML) is a light emitting layer, and an organic material layer including the electron transport material (ET) is provided between the light emitting layer and the negative electrode. In an exemplary embodiment, the light emitting layer and the organic material layer including the electron transport material (ET) are directly brought into contact with each other.

The organic material layer of the organic light emitting device of the present specification can also be composed of a single-layered structure, but can be composed of a multi-layered structure in which an organic material layer having two or more layers is stacked. Further, the organic light emitting device of the present application can have a structure including a hole injection layer, a hole transport layer, an electron blocking layer, a light emitting layer, a hole blocking layer, an electron transport layer, an electron injection layer, and the like as organic material layers. However, the structure of the organic light emitting device is not limited thereto, and can include a greater or fewer number of organic layers.

In an exemplary embodiment of the present specification, the organic light emitting device further includes one layer or two or more layers selected from the group consisting of a hole injection layer, a hole transport layer, an electron blocking layer, a light emitting layer, a hole blocking layer, an electron transport layer, and an electron injection layer.

In an exemplary embodiment of the present specification, the hole transport layer includes the hole transport material (HT), and is provided between the positive electrode and the light emitting layer.

In an exemplary embodiment of the present specification, the electron blocking layer includes the electron blocking material (EB), and is provided between the positive electrode and the light emitting layer.

In an exemplary embodiment of the present specification, the blue light emitting layer includes the blue light emitting dopant material (BD) and the blue light emitting host material (BH).

In an exemplary embodiment of the present specification, the green light emitting layer includes the light emitting host material (EML). In this case, the light emitting layer can further include a dopant, and the dopant is a phosphorescent dopant or fluorescent dopant.

In an exemplary embodiment of the present specification, the hole blocking layer includes the hole blocking material (HB), and is provided between the negative electrode and the light emitting layer.

In an exemplary embodiment of the present specification, the electron transport layer includes the electron transport material (ET), and is provided between the negative electrode and the light emitting layer.

In an exemplary embodiment of the present specification, the hole transport layer is a single layer of the hole transport material (HT), or other organic compounds are mixed and used.

In an exemplary embodiment of the present specification, the electron blocking layer is a single layer of the electron blocking material (EB), or other organic compounds are mixed and used.

In an exemplary embodiment of the present specification, the light emitting layer includes only the blue light emitting dopant material (BD) and the compound of Formula H, or other organic compounds are mixed and used.

In an exemplary embodiment of the present specification, the blue light emitting layer includes only the blue light emitting dopant material (BD) and the blue light emitting host material (BH), or other organic compounds are mixed and used.

In an exemplary embodiment of the present specification, the green light emitting layer includes only the light emitting host material (EML) and the dopant, or other organic compounds are mixed and used.

In an exemplary embodiment of the present specification, the hole blocking layer is a single layer of the hole blocking material (HB), or other organic compounds are mixed and used.

In an exemplary embodiment of the present specification, the electron transport layer is a single layer of the electron transport material (ET), or other organic compounds are mixed and used.

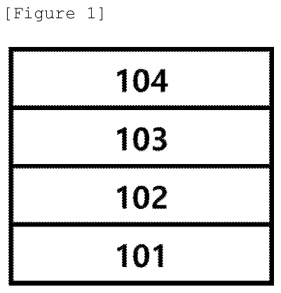

FIG. 1 exemplifies a structure of the organic light emitting device according to the present invention. The structure is a structure in which a substrate 101, a positive electrode 102, an organic material layer 103, and a negative electrode 104 are sequentially stacked.

### EXAMPLES

Hereinafter, the present specification will be described in detail with reference to Examples for specifically describing the present specification.

However, the Examples according to the present specification can be modified in various forms, and it is not interpreted that the scope of the present application is limited to the Examples described in detail below. The Examples of the present application are provided to explain the present specification more completely to a person with ordinary skill in the art.

Measurement of Reversibility (Iᵣ/I_{f})

Samples in which compounds were each dissolved in dimethylformamide (DMF) were prepared to obtain a cyclic voltammogram within an oxidation range or reduction range at 1 to 3 scan rates selected from 10 mV/s, 50 mV/s, 100 mV/s, 300 mV/s, and 500 mV/s. As an electrolyte, an electrolyte tert-butyl acetate (TBAC) was used, the EC-lab program was used, and the measurement was performed using the VSP model.

The values of the forward peak and the inverse peak are values obtained by setting peaks in the program and calculating the height from the baseline. A measured oxidation potential or reduction potential was calibrated by a calibration material ferrocene to obtain a HOMO or LUMO value.

HOMO=4.8−(the oxidation potential of ferrocene−the oxidation potential of a sample)

LUMO=4.8−(the oxidation potential of ferrocene−the reduction potential of a sample)

The reversibilities of the following Equation 1 were calculated by measuring the forward peak and the inverse peak of the following compounds with an oxidation range or reduction range, and are shown in the following Table 1.

Reversibility=Iᵣ/I_{f} <Equation 1>

In Equation 1, Iᵣ means the height of the inverse peak, and I_{f} means the height of the forward peak.

In the following Tables 1 to 11, the calculated LUMO or the calculated HOMO is the absolute value of LUMO or HOMO calculated by a time-dependent density functional theory (DFT) of the Gaussian program. The AC3 LUMO or AC3 HOMO is a HOMO or LUMO value measured by AC3.

As a hole transport material (HT), the following Compounds HTL1 to HTL5 were evaluated, and are shown in the following Table 1.

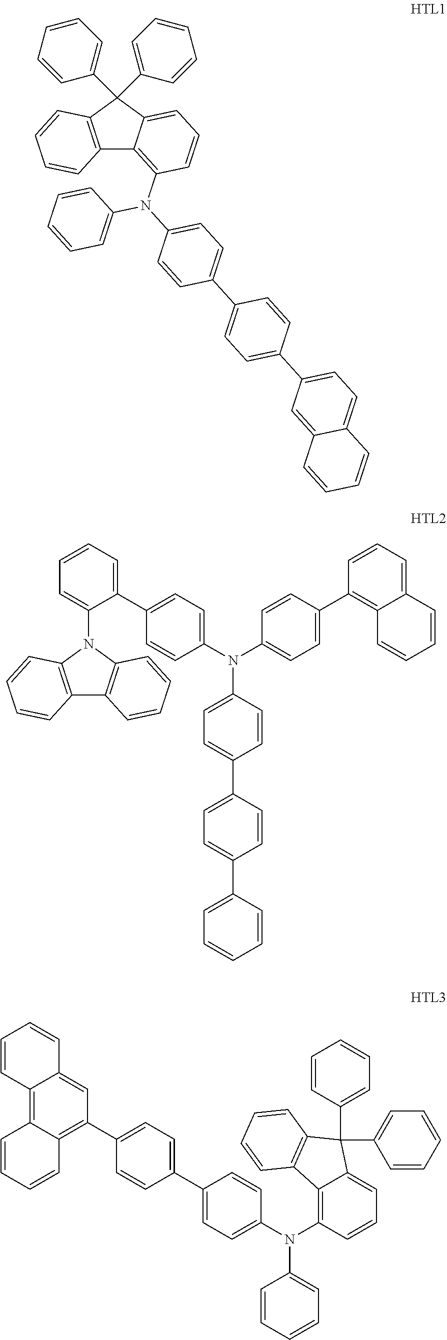 [SMILES: c1ccc(N(c2ccc(-c3ccc(-c4ccc5ccccc5c4)cc3)cc2)c2cccc3c2-c2ccccc2C3(c2ccccc2)c2ccccc2)cc1 ; c1ccc(-c2ccc(-c3ccc(N(c4ccc(-c5ccccc5-n5c6ccccc6c6ccccc65)cc4)c4ccc(-c5cccc6ccccc56)cc4)cc3)cc2)cc1 ; c1ccc(N(c2ccc(-c3ccc(-c4cc5ccccc5c5ccccc45)cc3)cc2)c2cccc3c2-c2ccccc2C3(c2ccccc2)c2ccccc2)cc1]

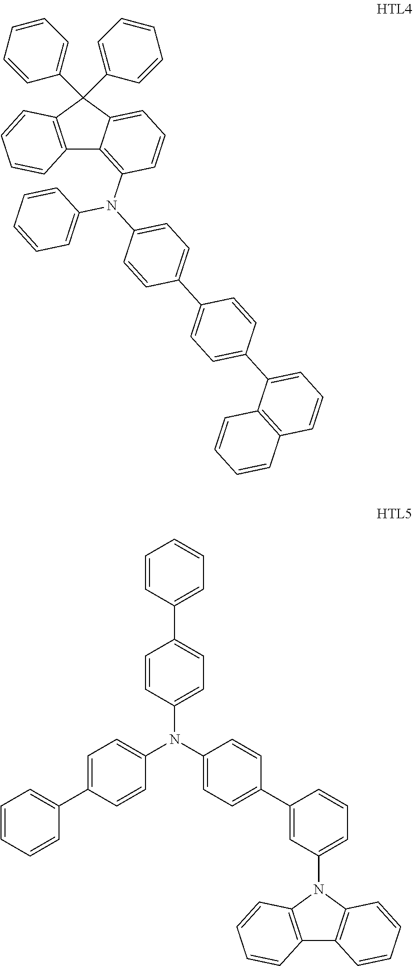 [SMILES: c1ccc(N(c2ccc(-c3ccc(-c4cccc5ccccc45)cc3)cc2)c2cccc3c2-c2ccccc2C3(c2ccccc2)c2ccccc2)cc1 ; c1ccc(-c2ccc(N(c3ccc(-c4ccccc4)cc3)c3ccc(-c4cccc(-n5c6ccccc6c6ccccc65)c4)cc3)cc2)cc1]

The service life shown in the following Tables 1 to 11 refers to the service life (%) of the device, the device structures are as follows, and only the applicable layer materials are varied in the respective examples.

Positive electrode (ITO)/Hole injection layer (106 Å, a weight ratio of Compounds HTL1 and P1 is 97:3)/Hole transport layer (1000 Å, Compound HTL1)/Electron blocking layer (40 Å, Compound HTL2)/Light emitting layer (190 Å, a weight ratio of Compounds BH and BD1 is 97:3)/Hole blocking layer (50 Å, Compound xETL)/Electron transport layer (250 Å, a weight ratio of Compounds ETL and LiQ is 50:50)/Electron injection layer (7 Å, LiQ)/Negative electrode (100 Å, a weight ratio of magnesium and silver is 10:1)/Capping layer (800 Å, Compound CPL)

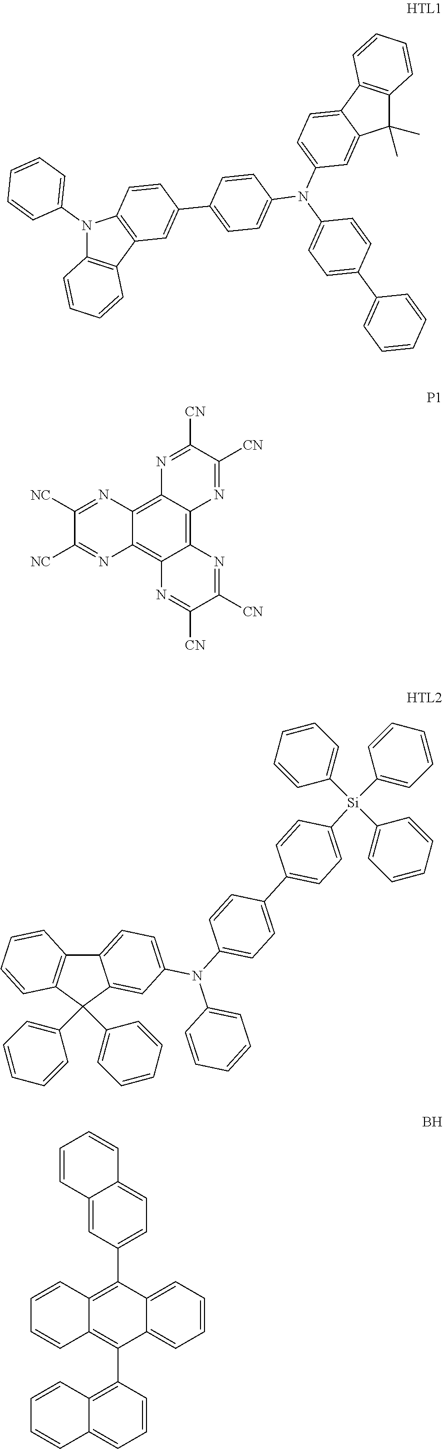

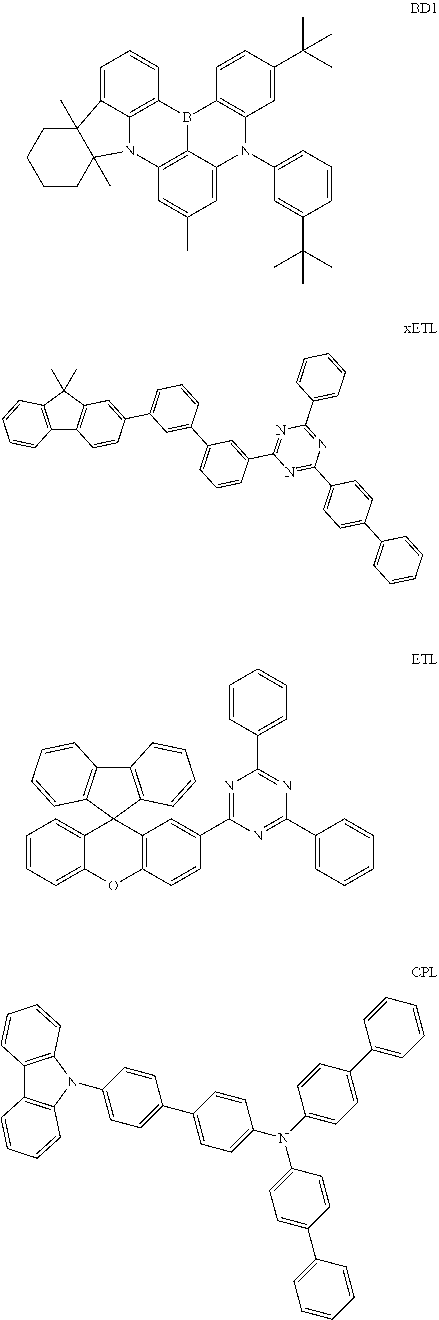 [SMILES: Cc1cc2c3c(c1)N1c4c(cccc4C4(C)CCCCC14C)B3c1ccc(C(C)(C)C)cc1N2c1cccc(C(C)(C)C)c1 ; CC1(C)c2ccccc2-c2ccc(-c3cccc(-c4cccc(-c5nc(-c6ccccc6)nc(-c6ccc(-c7ccccc7)cc6)n5)c4)c3)cc21 ; c1ccc(-c2nc(-c3ccccc3)nc(-c3ccc4c(c3)C3(c5ccccc5O4)c4ccccc4-c4ccccc43)n2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

TABLE 1

| | | | Oxidation | Reduction | |
|---|---|---|---|---|---|
| | | Service | Iᵣ/I_{f} in | Iᵣ/I_{f} in | Calculated |
| | Compound | life (%) | 100 mV/s | 10 mV/s | HOMO |
| Example 1-1 | HTL1 | 76 | 0.889 | 0.000 | 4.57 |
| Example 1-2 | HTL2 | 116 | 1.000 | 0.000 | 4.53 |
| Comparative | HTL3 | 45 | 0.763 | 0.000 | 4.59 |
| Example 1-1 | | | | | |
| Example 1-3 | HTL4 | 76 | 0.832 | 0.000 | 4.59 |
| Example 1-4 | HTL5 | 85 | 0.920 | 0.059 | 4.60 |

As an electron blocking material (EB), the following Compounds EB1 to EB25 were evaluated, and are shown in the following Table 2.

TABLE 2

| | |
|---|---|
| 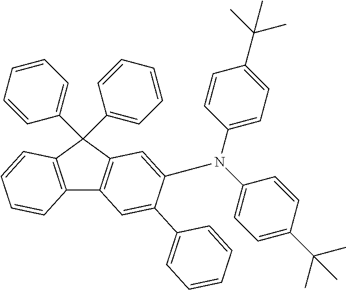 [SMILES: CC(C)(C)c1ccc(N(c2ccc(C(C)(C)C)cc2)c2cc3c(cc2-c2ccccc2)-c2ccccc2C3(c2ccccc2)c2ccccc2)cc1] | EB1 |
| 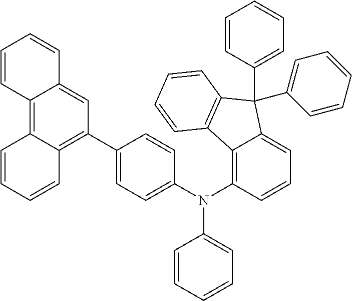 [SMILES: c1ccc(N(c2ccc(-c3cc4ccccc4c4ccccc34)cc2)c2cccc3c2-c2ccccc2C3(c2ccccc2)c2ccccc2)cc1] | EB2 |
| 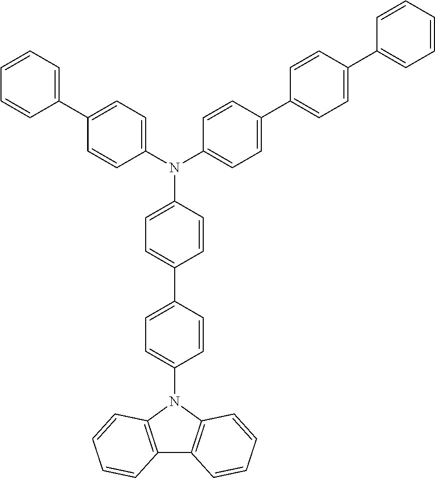 [SMILES: c1ccc(-c2ccc(-c3ccc(N(c4ccc(-c5ccccc5)cc4)c4ccc(-c5ccc(-n6c7ccccc7c7ccccc76)cc5)cc4)cc3)cc2)cc1] | EB3 |
| 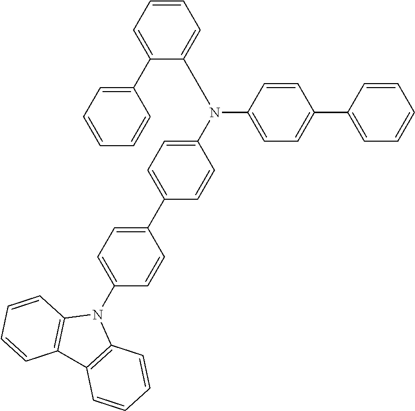 [SMILES: c1ccc(-c2ccc(N(c3ccc(-c4ccc(-n5c6ccccc6c6ccccc65)cc4)cc3)c3ccccc3-c3ccccc3)cc2)cc1] | EB4 |
| 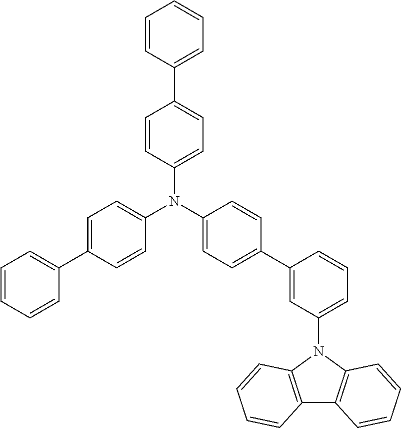 [SMILES: c1ccc(-c2ccc(N(c3ccc(-c4ccccc4)cc3)c3ccc(-c4cccc(-n5c6ccccc6c6ccccc65)c4)cc3)cc2)cc1] | EB5 |
| 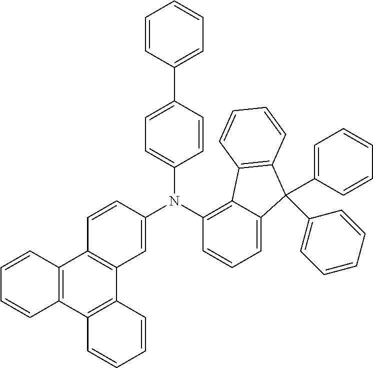 [SMILES: c1ccc(-c2ccc(N(c3ccc4c5ccccc5c5ccccc5c4c3)c3cccc4c3-c3ccccc3C4(c3ccccc3)c3ccccc3)cc2)cc1] | EB6 |
| 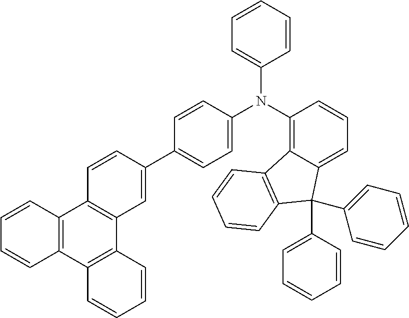 [SMILES: c1ccc(N(c2ccc(-c3ccc4c5ccccc5c5ccccc5c4c3)cc2)c2cccc3c2-c2ccccc2C3(c2ccccc2)c2ccccc2)cc1] | EB7 |
| 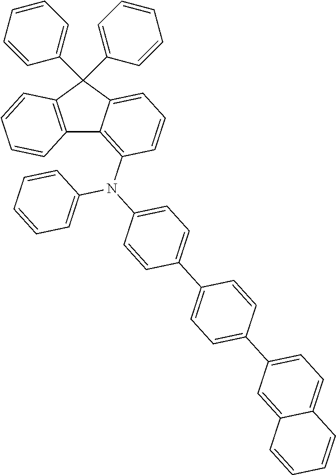 [SMILES: c1ccc(N(c2ccc(-c3ccc(-c4ccc5ccccc5c4)cc3)cc2)c2cccc3c2-c2ccccc2C3(c2ccccc2)c2ccccc2)cc1] | EB8 |
| 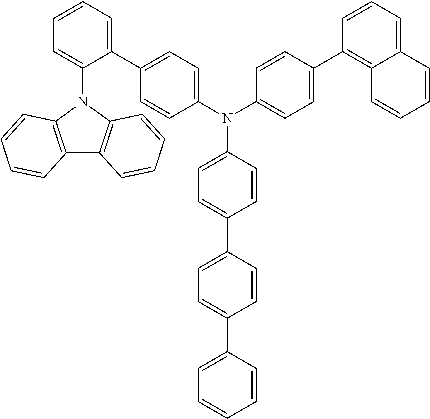 [SMILES: c1ccc(-c2ccc(-c3ccc(N(c4ccc(-c5ccccc5-n5c6ccccc6c6ccccc65)cc4)c4ccc(-c5cccc6ccccc56)cc4)cc3)cc2)cc1] | EB9 |
| 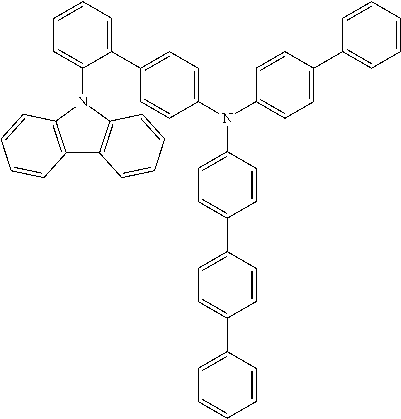 [SMILES: c1ccc(-c2ccc(-c3ccc(N(c4ccc(-c5ccccc5)cc4)c4ccc(-c5ccccc5-n5c6ccccc6c6ccccc65)cc4)cc3)cc2)cc1] | EB10 |
| 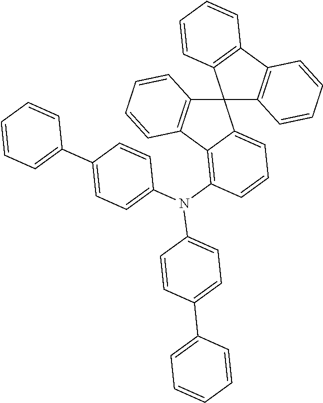 [SMILES: c1ccc(-c2ccc(N(c3ccc(-c4ccccc4)cc3)c3cccc4c3-c3ccccc3C43c4ccccc4-c4ccccc43)cc2)cc1] | EB11 |
| 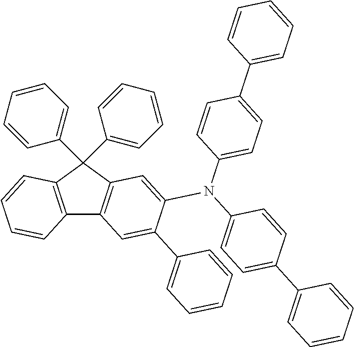 [SMILES: c1ccc(-c2ccc(N(c3ccc(-c4ccccc4)cc3)c3cc4c(cc3-c3ccccc3)-c3ccccc3C4(c3ccccc3)c3ccccc3)cc2)cc1] | EB12 |
| 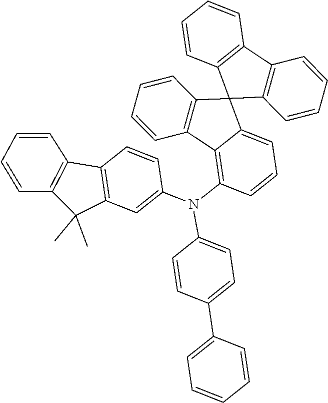 [SMILES: CC1(C)c2ccccc2-c2ccc(N(c3ccc(-c4ccccc4)cc3)c3cccc4c3-c3ccccc3C43c4ccccc4-c4ccccc43)cc21] | EB13 |
| 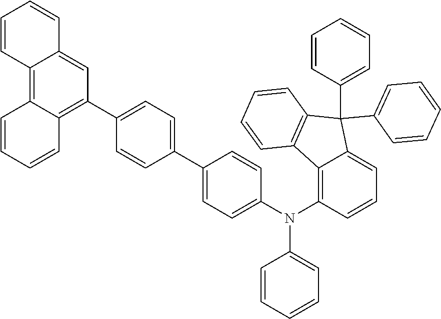 [SMILES: c1ccc(N(c2ccc(-c3ccc(-c4cc5ccccc5c5ccccc45)cc3)cc2)c2cccc3c2-c2ccccc2C3(c2ccccc2)c2ccccc2)cc1] | EB14 |
| 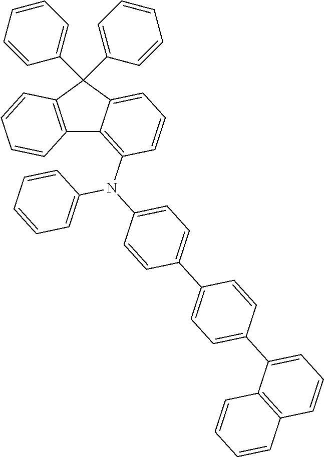 [SMILES: c1ccc(N(c2ccc(-c3ccc(-c4cccc5ccccc45)cc3)cc2)c2cccc3c2-c2ccccc2C3(c2ccccc2)c2ccccc2)cc1] | EB15 |
| 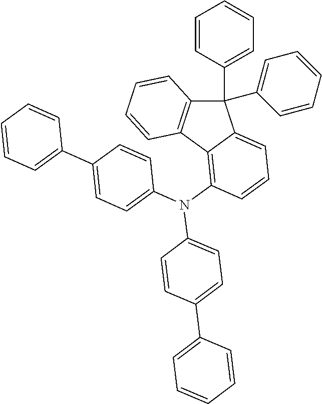 [SMILES: c1ccc(-c2ccc(N(c3ccc(-c4ccccc4)cc3)c3cccc4c3-c3ccccc3C4(c3ccccc3)c3ccccc3)cc2)cc1] | EB16 |
| 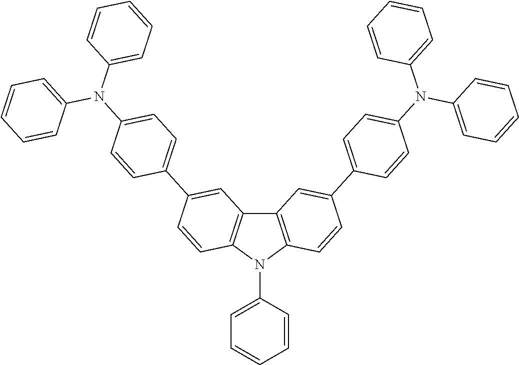 [SMILES: c1ccc(N(c2ccccc2)c2ccc(-c3ccc4c(c3)c3cc(-c5ccc(N(c6ccccc6)c6ccccc6)cc5)ccc3n4-c3ccccc3)cc2)cc1] | EB17 |
| 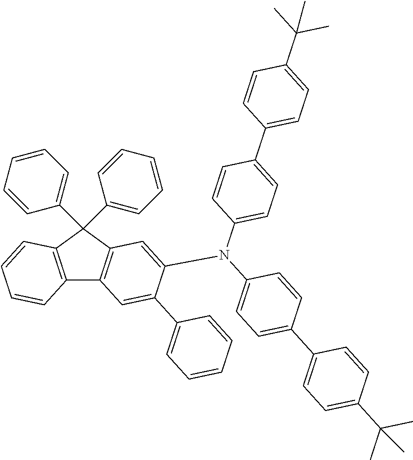 [SMILES: CC(C)(C)c1ccc(-c2ccc(N(c3ccc(-c4ccc(C(C)(C)C)cc4)cc3)c3cc4c(cc3-c3ccccc3)-c3ccccc3C4(c3ccccc3)c3ccccc3)cc2)cc1] | EB18 |
| 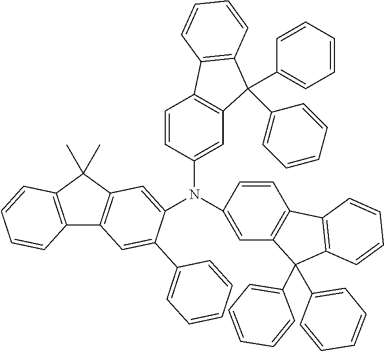 [SMILES: CC1(C)c2ccccc2-c2cc(-c3ccccc3)c(N(c3ccc4c(c3)C(c3ccccc3)(c3ccccc3)c3ccccc3-4)c3ccc4c(c3)C(c3ccccc3)(c3ccccc3)c3ccccc3-4)cc21] | EB19 |
| 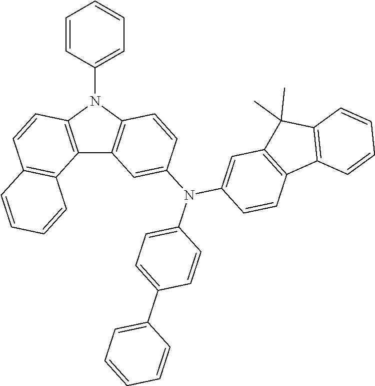 [SMILES: CC1(C)c2ccccc2-c2ccc(N(c3ccc(-c4ccccc4)cc3)c3ccc4c(c3)c3c5ccccc5ccc3n4-c3ccccc3)cc21] | EB20 |
| 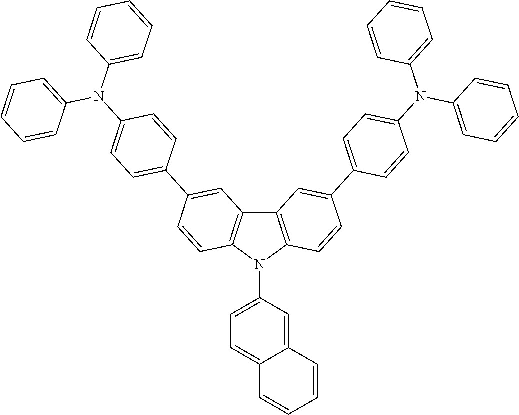 [SMILES: c1ccc(N(c2ccccc2)c2ccc(-c3ccc4c(c3)c3cc(-c5ccc(N(c6ccccc6)c6ccccc6)cc5)ccc3n4-c3ccc4ccccc4c3)cc2)cc1] | EB21 |
| 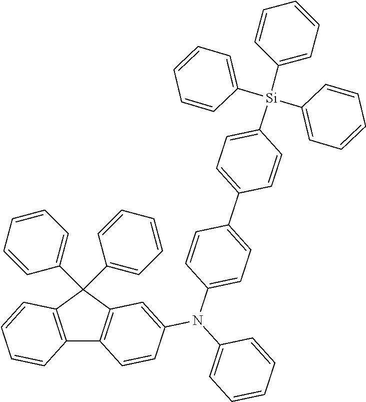 [SMILES: c1ccc(N(c2ccc(-c3ccc([Si](c4ccccc4)(c4ccccc4)c4ccccc4)cc3)cc2)c2ccc3c(c2)C(c2ccccc2)(c2ccccc2)c2ccccc2-3)cc1] | EB22 |
| 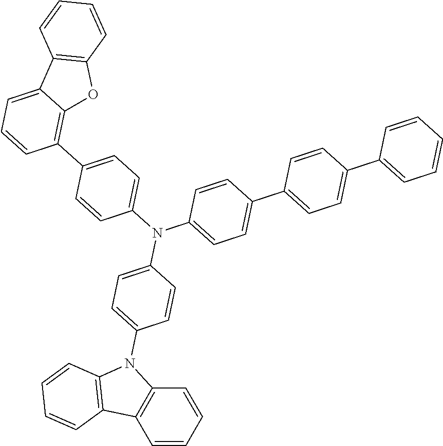 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1] | EB23 |
| 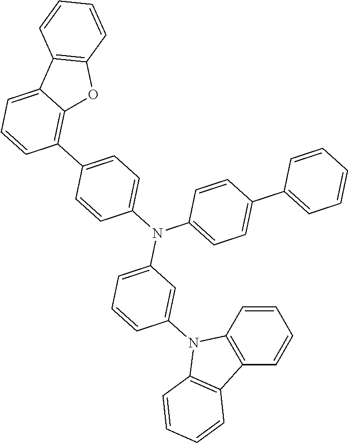 [SMILES: c1ccc(-c2ccc(N(c3ccc(-c4cccc5c4oc4ccccc45)cc3)c3cccc(-n4c5ccccc5c5ccccc54)c3)cc2)cc1] | EB24 |
| 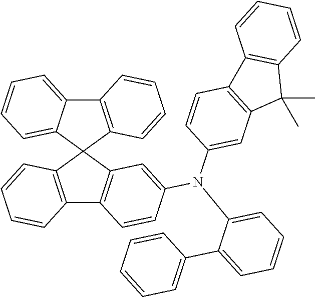 [SMILES: CC1(C)c2ccccc2-c2ccc(N(c3ccc4c(c3)C3(c5ccccc5-c5ccccc53)c3ccccc3-4)c3ccccc3-c3ccccc3)cc21] | EB25 |

| | | | Oxidation Ir/If | Calculated |
|---|---|---|---|---|
| | Name of material | Service life (%) | in 100 mV/s | HOMO |
| Example 2-1 | EB1 | 123 | 0.976 | 4.31 |
| Comparative | EB2 | 6 | 0.300 | 4.63 |
| Example 2-1 | | | | |
| Example-2-2 | EB3 | 135 | 0.979 | 4.58 |
| Example 2-3 | EB4 | 110 | 0.948 | 4.59 |
| Example 2-4 | EB5 | 132 | 0.968 | 4.6 |
| Example 2-5 | EB6 | 117 | 1.001 | 4.6 |
| Comparative | EB7 | 32 | 0.324 | 4.61 |
| Example 2-2 | | | | |
| Example 2-6 | EB8 | 105 | 0.889 | 4.57 |
| Example 2-7 | EB9 | 135 | 1.000 | 4.53 |
| Example 2-8 | EB10 | 140 | 0.980 | 4.5 |
| Example 2-9 | EB11 | 113 | 0.970 | 4.58 |
| Example 2-10 | EB12 | 136 | 1.000 | 4.45 |
| Example 2-11 | EB13 | 143 | 0.986 | 4.5 |
| Example 2-12 | EB14 | 100 | 0.772 | 4.59 |
| Example 2-13 | EB15 | 110 | 0.843 | 4.59 |
| Comparative | EB16 | 62 | 0.500 | 4.57 |
| Example 2-3 | | | | |
| Example 2-14 | EB17 | 160 | 1.000 | 4.35 |
| Example 2-15 | EB18 | 154 | 1.000 | 4.37 |
| Example 2-16 | EB19 | 154 | 0.999 | 4.37 |
| Example 2-17 | EB20 | 143 | 0.900 | 4.26 |
| Example 2-18 | EB21 | 150 | 1.000 | 4.35 |
| Example 2-19 | EB22 | 145 | 1.000 | 4.45 |
| Example 2-20 | EB23 | 140 | 0.991 | 4.61 |
| Example 2-21 | EB24 | 150 | 0.990 | 4.43 |
| Example 2-22 | EB26 | 160 | 0.984 | 4.36 |

The service life of each of the devices including both the hole transport material (HT) and the electron blocking material (EB) was measured, and is shown in the following Table 3. The “difference” in the following Table 3 refers to a value of (the oxidation stability of the hole transport material (HT)−the oxidation stability of the electron blocking material (EB)).

TABLE 3

| | HT | | EB | | | |
|---|---|---|---|---|---|---|
| | | Oxidation | | Oxidation | | Service |
| | Com- | Ir/If in | Com- | Ir/If in | Dif- | life |
| | pound | 100 mV/s | pound | 100 mV/s | ference | (%) |
| Example 3-1 | HTL1 | 0.889 | EB1 | 0.976 | −0.087 | 82 |
| Example 3-2 | HTL1 | 0.889 | EB3 | 0.979 | −0.090 | 112 |
| Example 3-3 | HTL1 | 0.889 | EB4 | 0.948 | −0.059 | 78 |
| Example 3-4 | HTL1 | 0.889 | EB5 | 0.968 | −0.079 | 110 |
| Example 3-5 | HTL1 | 0.889 | EB6 | 1.001 | −0.112 | 88 |
| Example 3-6 | HTL1 | 0.889 | EB8 | 0.889 | 0.000 | 84 |
| Example 3-7 | HTL1 | 0.889 | EB9 | 1.000 | −0.111 | 107 |
| Comparative | HTL1 | 0.889 | EB2 | 0.300 | 0.589 | 5 |
| Example 3-1 | | | | | | |
| Example 3-8 | HTL2 | 1.000 | EB1 | 0.976 | 0.024 | 125 |
| Example 3-9 | HTL2 | 1.000 | EB3 | 0.979 | 0.021 | 169 |
| Example 3-10 | HTL2 | 1.000 | EB4 | 0.948 | 0.052 | 113 |
| Example 3-11 | HTL2 | 1.000 | EBS | 0.968 | 0.032 | 163 |
| Example 3-12 | HTL2 | 1.000 | EB6 | 1.001 | −0.001 | 140 |
| Example 3-13 | HTL2 | 1.000 | EB8 | 0.889 | 0.111 | 120 |
| Example 3-14 | HTL2 | 1.000 | EB9 | 1.000 | 0.000 | 159 |
| Comparative | HTL2 | 1.000 | EB7 | 0.324 | 0.676 | 35 |
| Example 3-2 | | | | | | |
| Example 3-15 | HTL4 | 0.832 | EB1 | 0.976 | −0.144 | 80 |
| Example 3-16 | HTL4 | 0.832 | EB3 | 0.979 | −0.147 | 109 |
| Example 3-17 | HTL4 | 0.832 | EB4 | 0.948 | −0.116 | 80 |
| Example 3-18 | HTL4 | 0.832 | EB5 | 0.968 | −0.136 | 108 |
| Example 3-19 | HTL4 | 0.832 | EB6 | 1.001 | −0.169 | 87 |
| Example 3-20 | HTL4 | 0.832 | EB8 | 0.889 | −0.057 | 78 |
| Example 3-21 | HTL4 | 0.832 | EB9 | 1.000 | −0.168 | 100 |
| Comparative | HTL4 | 0.832 | EB7 | 0.324 | 0.508 | 24 |
| Example 3-3 | | | | | | |
| Example 3-22 | HTL5 | 0.92 | EB1 | 0.976 | −0.056 | 91 |
| Example 3-23 | HTL5 | 0.92 | EB3 | 0.979 | −0.059 | 127 |
| Example 3-24 | HTL5 | 0.92 | EB4 | 0.948 | −0.028 | 84 |
| Example 3-25 | HTL5 | 0.92 | EBS | 0.968 | −0.048 | 123 |
| Example 3-26 | HTL5 | 0.92 | EB6 | 1.001 | −0.081 | 100 |
| Example 3-27 | HTL5 | 0.92 | EB8 | 0.889 | 0.031 | 89 |
| Example 3-28 | HTL5 | 0.92 | EB9 | 1.000 | −0.080 | 120 |
| Comparative | HTL5 | 0.92 | EB7 | 0.324 | 0.596 | 30 |
| Example 3-4 | | | | | | |

As a light emitting dopant material (BD), the following compounds were evaluated, and are shown in the following Table 4. The compounds are blue light emitting dopants, and the stability of the (−) radical is a factor that affects the service life.

TABLE 4

| | |
|---|---|
| 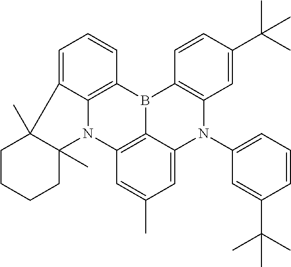 [SMILES: Cc1cc2c3c(c1)N1c4c(cccc4C4(C)CCCCC14C)B3c1ccc(C(C)(C)C)cc1N2c1cccc(C(C)(C)C)c1] | BD1 |
| 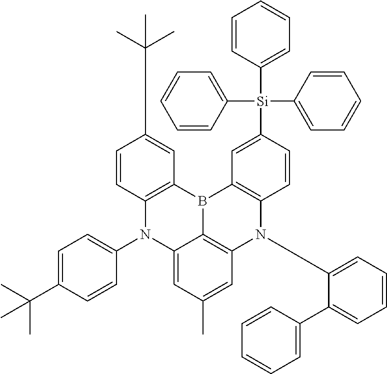 [SMILES: Cc1cc2c3c(c1)N(c1ccccc1-c1ccccc1)c1ccc([Si](c4ccccc4)(c4ccccc4)c4ccccc4)cc1B3c1cc(C(C)(C)C)ccc1N2c1ccc(C(C)(C)C)cc1] | BD2 |
| 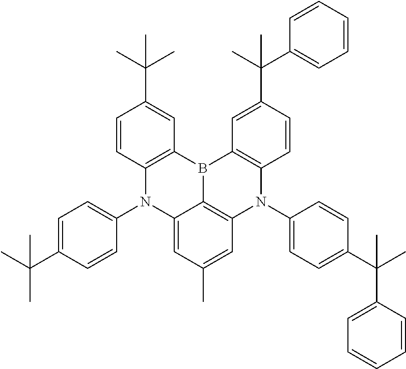 [SMILES: Cc1cc2c3c(c1)N(c1ccc(C(C)(C)c4ccccc4)cc1)c1ccc(C(C)(C)c4ccccc4)cc1B3c1cc(C(C)(C)C)ccc1N2c1ccc(C(C)(C)C)cc1] | BD3 |
| 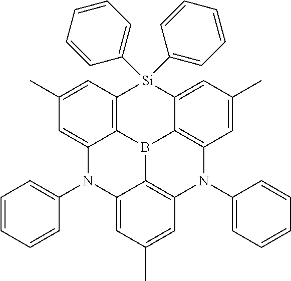 [SMILES: Cc1cc2c3c(c1)N(c1ccccc1)c1cc(C)cc4c1B3c1c(cc(C)cc1[Si]4(c1ccccc1)c1ccccc1)N2c1ccccc1] | BD4 |
| 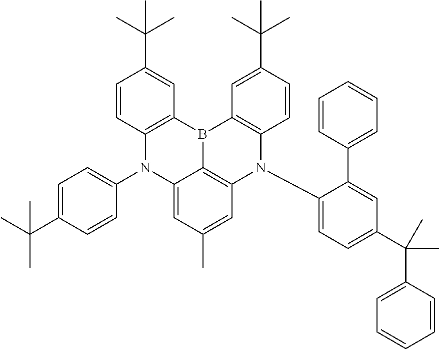 [SMILES: Cc1cc2c3c(c1)N(c1ccc(C(C)(C)c4ccccc4)cc1-c1ccccc1)c1ccc(C(C)(C)C)cc1B3c1cc(C(C)(C)C)ccc1N2c1ccc(C(C)(C)C)cc1] | BD5 |
| 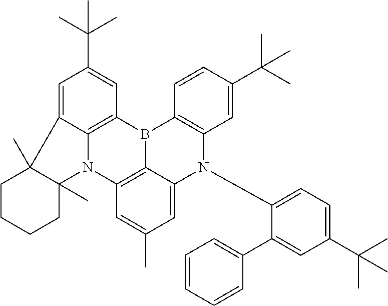 [SMILES: Cc1cc2c3c(c1)N1c4c(cc(C(C)(C)C)cc4C4(C)CCCCC14C)B3c1ccc(C(C)(C)C)cc1N2c1ccc(C(C)(C)C)cc1-c1ccccc1] | BD6 |
| 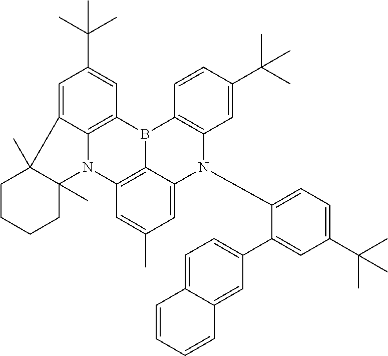 [SMILES: Cc1cc2c3c(c1)N1c4c(cc(C(C)(C)C)cc4C4(C)CCCCC14C)B3c1ccc(C(C)(C)C)cc1N2c1ccc(C(C)(C)C)cc1-c1ccc2ccccc2c1] | BD7 |
| 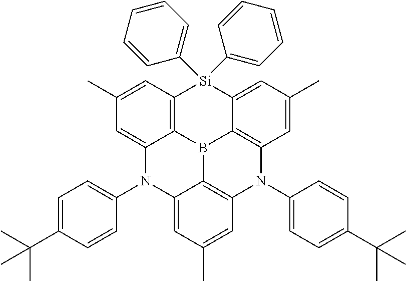 [SMILES: Cc1cc2c3c(c1)N(c1ccc(C(C)(C)C)cc1)c1cc(C)cc4c1B3c1c(cc(C)cc1[Si]4(c1ccccc1)c1ccccc1)N2c1ccc(C(C)(C)C)cc1] | BD8 |
| 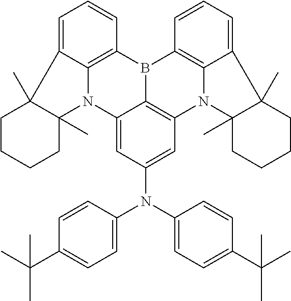 [SMILES: CC(C)(C)c1ccc(N(c2ccc(C(C)(C)C)cc2)c2cc3c4c(c2)N2c5c(cccc5C5(C)CCCCC25C)B4c2cccc4c2N3C2(C)CCCCC42C)cc1] | BD10 |
| 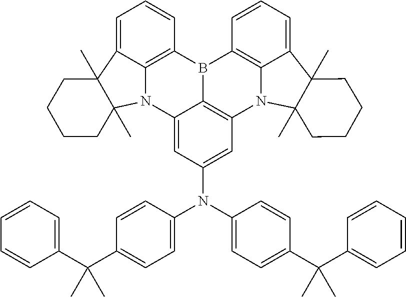 [SMILES: CC(C)(c1ccccc1)c1ccc(N(c2ccc(C(C)(C)c3ccccc3)cc2)c2cc3c4c(c2)N2c5c(cccc5C5(C)CCCCC25C)B4c2cccc4c2N3C2(C)CCCCC42C)cc1] | BD11 |
| 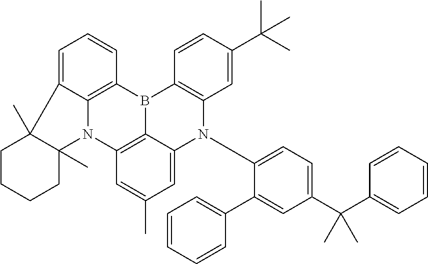 [SMILES: Cc1cc2c3c(c1)N1c4c(cccc4C4(C)CCCCC14C)B3c1ccc(C(C)(C)C)cc1N2c1ccc(C(C)(C)c2ccccc2)cc1-c1ccccc1] | BD12 |
| 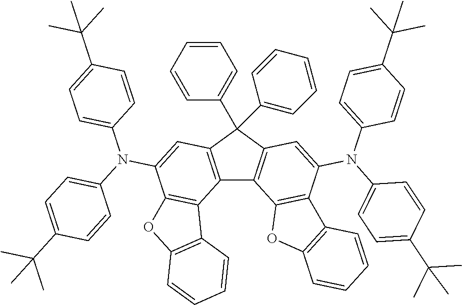 [SMILES: CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2oc2ccccc24)C3(c2ccccc2)c2ccccc2)cc1] | BD13 |
| 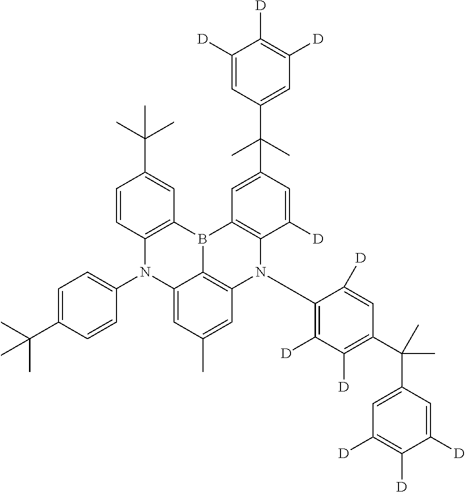 [SMILES: [2H]c1cc(C(C)(C)c2cc([2H])c3c(c2)B2c4cc(C(C)(C)C)ccc4N(c4ccc(C(C)(C)C)cc4)c4cc(C)cc(c42)N3c2c([2H])cc(C(C)(C)c3cc([2H])c([2H])c([2H])c3)c([2H])c2[2H])cc([2H])c1[2H]] | BD14 |
| 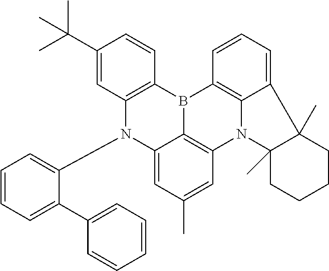 [SMILES: Cc1cc2c3c(c1)N1c4c(cccc4C4(C)CCCCC14C)B3c1ccc(C(C)(C)C)cc1N2c1ccccc1-c1ccccc1] | BD15 |
| 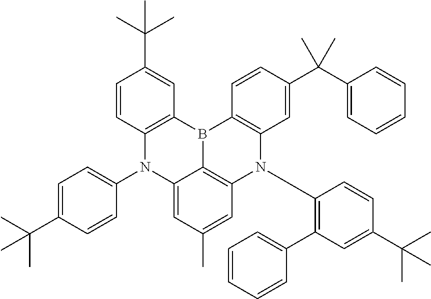 [SMILES: Cc1cc2c3c(c1)N(c1ccc(C(C)(C)C)cc1-c1ccccc1)c1cc(C(C)(C)c4ccccc4)ccc1B3c1cc(C(C)(C)C)ccc1N2c1ccc(C(C)(C)C)cc1] | BD16 |
| 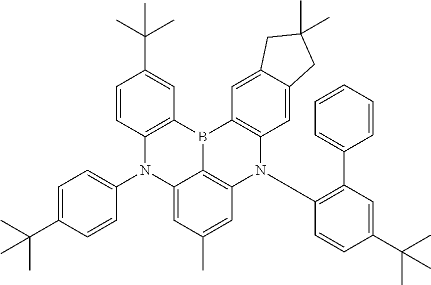 [SMILES: Cc1cc2c3c(c1)N(c1ccc(C(C)(C)C)cc1-c1ccccc1)c1cc4c(cc1B3c1cc(C(C)(C)C)ccc1N2c1ccc(C(C)(C)C)cc1)CC(C)(C)C4] | BD17 |
| 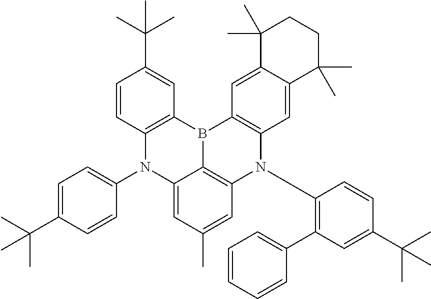 [SMILES: Cc1cc2c3c(c1)N(c1ccc(C(C)(C)C)cc1-c1ccccc1)c1cc4c(cc1B3c1cc(C(C)(C)C)ccc1N2c1ccc(C(C)(C)C)cc1)C(C)(C)CCC4(C)C] | BD18 |
| 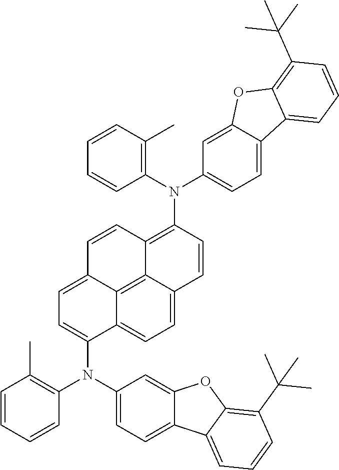 [SMILES: Cc1ccccc1N(c1ccc2c(c1)oc1c(C(C)(C)C)cccc12)c1ccc2ccc3c(N(c4ccc5c(c4)oc4c(C(C)(C)C)cccc45)c4ccccc4C)ccc4ccc1c2c43] | BD20 |
| 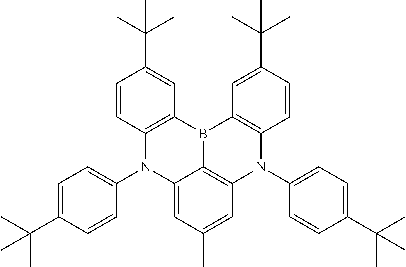 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1] | BD21 |
| 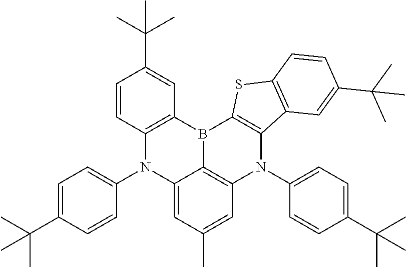 [SMILES: Cc1cc2c3c(c1)N(c1ccc(C(C)(C)C)cc1)c1c(sc4ccc(C(C)(C)C)cc14)B3c1cc(C(C)(C)C)ccc1N2c1ccc(C(C)(C)C)cc1] | BD22 |
| 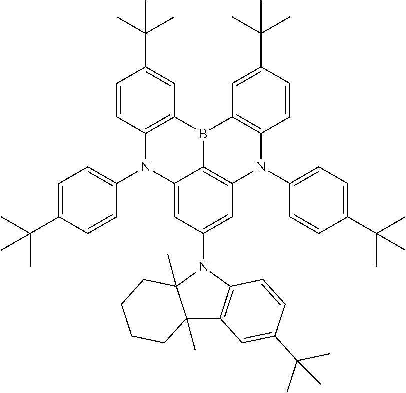 [SMILES: CC(C)(C)c1ccc(N2c3ccc(C(C)(C)C)cc3B3c4cc(C(C)(C)C)ccc4N(c4ccc(C(C)(C)C)cc4)c4cc(N5c6ccc(C(C)(C)C)cc6C6(C)CCCCC56C)cc2c43)cc1] | BD23 |
| 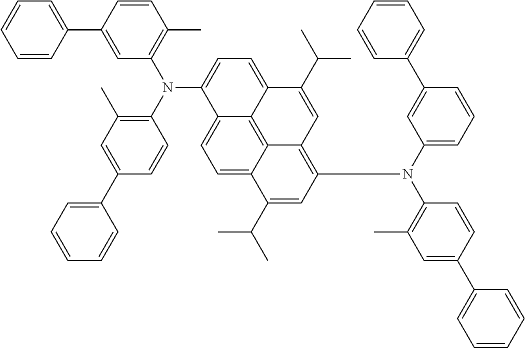 [SMILES: Cc1cc(-c2ccccc2)ccc1N(c1cccc(-c2ccccc2)c1)c1cc(C(C)C)c2ccc3c(N(c4ccc(-c5ccccc5)cc4C)c4cc(-c5ccccc5)ccc4C)ccc4c(C(C)C)cc1c2c43] | BD24 |
| 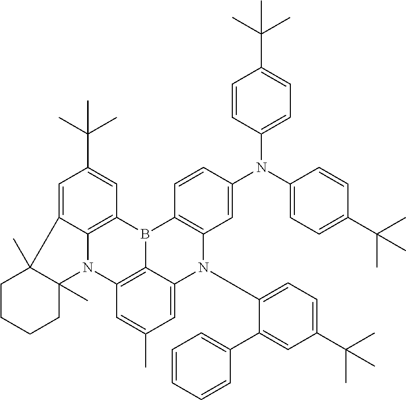 [SMILES: Cc1cc2c3c(c1)N1c4c(cc(C(C)(C)C)cc4C4(C)CCCCC14C)B3c1ccc(N(c3ccc(C(C)(C)C)cc3)c3ccc(C(C)(C)C)cc3)cc1N2c1ccc(C(C)(C)C)cc1-c1ccccc1] | BD25 |
| 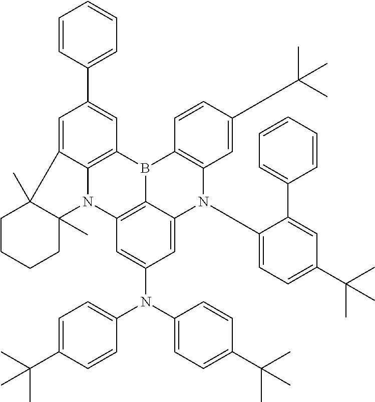 [SMILES: CC(C)(C)c1ccc(N(c2ccc(C(C)(C)C)cc2)c2cc3c4c(c2)N2c5c(cc(-c6ccccc6)cc5C5(C)CCCCC25C)B4c2ccc(C(C)(C)C)cc2N3c2ccc(C(C)(C)C)cc2-c2ccccc2)cc1] | BD26 |

| | | | | Reduction | | |
|---|---|---|---|---|---|---|
| | | | | Iᵣ/I_{f} | Device data | |
| | | AC3 | | in 100 | (measured) | |
| | Compound | HOMO | LUMO | mV/s | Efficiency | life |
| Example 4-1 | BD1 | 5.25 | 2.56 | 0.860 | 91 | 155 |
| Example 4-2 | BD2 | 5.42 | 2.74 | 0.636 | 100 | 100 |
| Comparative | BD3 | 5.46 | 2.79 | 0.000 | 119 | 64 |
| Example 4-1 | | | | | | |
| Comparative | BD4 | 5.52 | 2.87 | 1.000 | 96 | 73 |
| Example 4-2 | | | | | | |
| Example 4-3 | BD5 | 5.46 | 2.78 | 0.682 | 113 | 85 |
| Example 4-4 | BD6 | 5.16 | 2.48 | 0.780 | 100 | 198 |
| Example 4-5 | BD7 | 5.2 | 2.52 | 0.600 | 96 | 162 |
| Example 4-6 | BD8 | 5.31 | 2.67 | 0.000 | 112 | 96 |
| Example 4-7 | BD10 | 5.22 | 2.52 | 0.800 | 100 | 210 |
| Example 4-8 | BD11 | 5.32 | 2.644 | 0.800 | 103 | 147 |
| Example 4-9 | BD12 | 5.38 | 2.691 | 0.020 | 104 | 120 |
| Example 4-10 | BD13 | 5.31 | 2.58 | 0.870 | 112 | 160 |
| Comparative | BD14 | 5.46 | 2.79 | 0.000 | 119 | 62 |
| Example 4-3 | | | | | | |
| Example 4-11 | BD15 | 5.27 | 2.595 | 0.000 | 103 | 130 |
| Example 4-12 | BD16 | 5.46 | 2.768 | 0.250 | 106 | 85 |
| Example 4-13 | BD17 | 5.44 | 2.75 | 0.840 | 108 | 100 |
| Example 4-14 | BD18 | 5.43 | 2.75 | 0.856 | 111 | 120 |
| Example 4-16 | BD20 | 5.59 | 2.78 | 1.000 | 110 | 110 |
| Example 4-17 | BD21 | 5.34 | 2.64 | 0.000 | 84 | 110 |
| Example 4-18 | BD22 | 5.35 | 2.65 | 0.880 | 110 | 150 |
| Example 4-19 | BD24 | 5.45 | 2.77 | 1.000 | 110 | 110 |
| Example 4-20 | BD25 | 5.23 | 2.55 | 0.839 | 113 | 179 |
| Example 4-21 | BD26 | 5.12 | 2.483 | 0.100 | 104 | 182 |

As a blue light emitting host material (BH), the following compounds were evaluated, and are shown in the following Table 5. The dipole moment (D.M) (Debye) was calculated using a quantum chemical calculation program Gaussian 03 manufactured by U.S. Gaussian Corporation, and a density functional theory (DFT) was used and the calculated value of the triplet energy was obtained by the time-dependent-density functional theory (TD-DFT) with respect to a structure optimized using B3LYP as a functional and 6-31G* as a basis function. Q1 in the following Table 5 is the value of [1.34×(dipole moment)−0.293].

TABLE 5

| | |
|---|---|
| 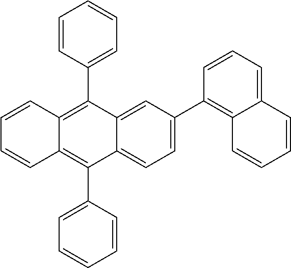 [SMILES: c1ccc(-c2c3ccccc3c(-c3ccccc3)c3cc(-c4cccc5ccccc45)ccc23)cc1] | BH1 |
| 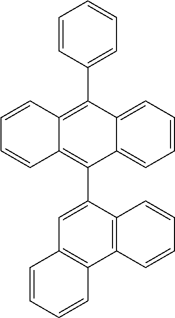 [SMILES: c1ccc(-c2c3ccccc3c(-c3cc4ccccc4c4ccccc34)c3ccccc23)cc1] | BH2 |
| 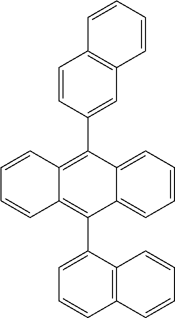 [SMILES: c1ccc2cc(-c3c4ccccc4c(-c4cccc5ccccc45)c4ccccc34)ccc2c1] | BH3 |
| 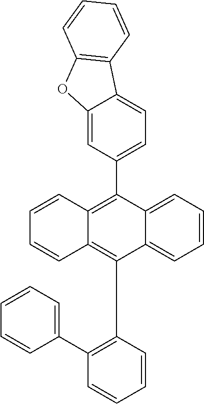 [SMILES: c1ccc(-c2ccccc2-c2c3ccccc3c(-c3ccc4c(c3)oc3ccccc34)c3ccccc23)cc1] | BH4 |
| 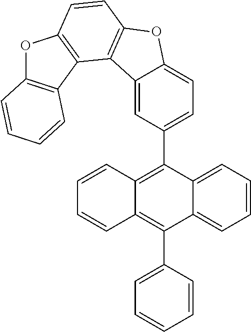 [SMILES: c1ccc(-c2c3ccccc3c(-c3ccc4oc5ccc6oc7ccccc7c6c5c4c3)c3ccccc23)cc1] | BH5 |
| 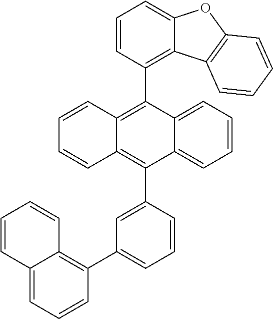 [SMILES: c1cc(-c2cccc3ccccc23)cc(-c2c3ccccc3c(-c3cccc4oc5ccccc5c34)c3ccccc23)c1] | BH6 |
| 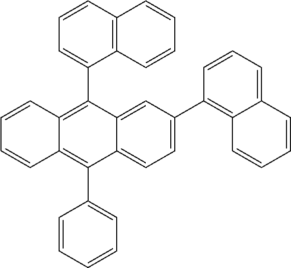 [SMILES: c1ccc(-c2c3ccccc3c(-c3cccc4ccccc34)c3cc(-c4cccc5ccccc45)ccc23)cc1] | BH7 |
| 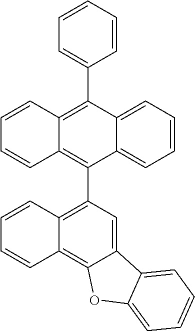 [SMILES: c1ccc(-c2c3ccccc3c(-c3cc4c5ccccc5oc4c4ccccc34)c3ccccc23)cc1] | BH8 |
| 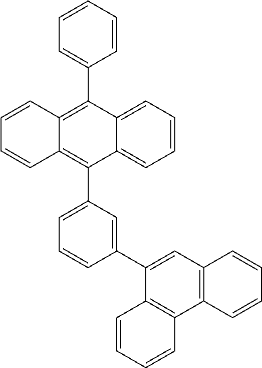 [SMILES: c1ccc(-c2c3ccccc3c(-c3cccc(-c4cc5ccccc5c5ccccc45)c3)c3ccccc23)cc1] | BH10 |
| 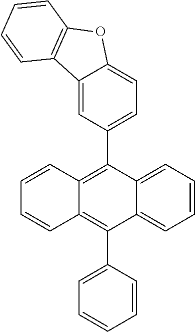 [SMILES: c1ccc(-c2c3ccccc3c(-c3ccc4oc5ccccc5c4c3)c3ccccc23)cc1] | BH11 |
| 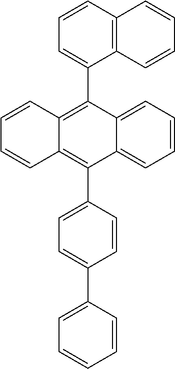 [SMILES: c1ccc(-c2ccc(-c3c4ccccc4c(-c4cccc5ccccc45)c4ccccc34)cc2)cc1] | BH12 |

| | | | | Oxidation | Reduction | | |
|---|---|---|---|---|---|---|---|
| | | | | Iᵣ/I_{f} | Iᵣ/I_{f} | | |
| | | | AC3 | in 500 | in 10 | Service | |
| | Compound | Dipole moment | LUMO | mV/s | mV/s | life (%) | Q1 |
| Example 5-1 | BH1 | 0.16 | 3.055 | 0.510 | 0.951 | 120 | −0.079 |
| Example 5-2 | BH2 | 0.18 | 3.090 | 0.605 | 0.95 | 134 | −0.052 |
| Example 5-3 | BH3 | 0.17 | 2.960 | 0.857 | 1.085 | 140 | −0.065 |
| Example 5-4 | BH4 | 0.85 | 3.003 | 0.930 | 0.951 | 95 | 0.846 |
| Comparative | BH5 | 1.19 | 2.962 | 0.855 | 0.987 | 42 | 1.302 |
| Example 5-1 | | | | | | | |
| Comparative | BH6 | 0.96 | 3.012 | 0.773 | 0.981 | 65 | 0.993 |
| Example 5-2 | | | | | | | |
| Example 5-5 | BH7 | 0.3 | 3.080 | 0.205 | 0.972 | 86 | 0.109 |
| Comparative | BH8 | 0.73 | 2.950 | 0.453 | 0.988 | 69 | 0.685 |
| Example 5-3 | | | | | | | |
| Example 5-7 | BH10 | 0.12 | 2.940 | 0.797 | 0.977 | 162.5 | −0.132 |
| Example 5-8 | BH11 | 0.75 | 0.72 | 0.72 | 0.960 | 90 | 0.712 |
| Example 5-9 | BH12 | 0.18 | 2.925 | 0.417 | 0.968 | 99 | −0.052 |

The service life of each of the devices including both the blue light emitting dopant material (BD) and the blue light emitting host material (BH) was measured, and is shown in the following Table 6. “LUMO difference” in the following Table 6 refers to the value of (the LUMO of the blue light emitting host material (BH)−the LUMO of blue light emitting dopant material (BD)). In the following Table 6, D.M means a dipole moment.

TABLE 6

| | Blue light emitting host (BH) | | | | Blue light emitting dopant (BD) | | | | |
|---|---|---|---|---|---|---|---|---|---|
| | | | Oxidation | | | | Reduction | Device | |
| | | | Iᵣ/I_{f} | | | | Iᵣ/I_{f} | | Service |
| | | AC3 | in 500 | | | AC3 | in 100 | LUMO | life |
| | Compound | LUMO | mV/s | D.M | Compound | LUMO | mV/s | Difference | (%) |
| Example 6-2 | BH1 | 3.055 | 0.510 | 0.16 | BD13 | 2.58 | 0.870 | 0.475 | 180 |
| Example 6-3 | BH1 | 3.055 | 0.510 | 0.16 | BD20 | 2.78 | 1.000 | 0.275 | 130 |
| Example 6-4 | BH1 | 3.055 | 0.510 | 0.16 | BD21 | 2.64 | 0.000 | 0.415 | 135 |
| Example 6-5 | BH1 | 3.055 | 0.510 | 0.16 | BD22 | 2.65 | 0.880 | 0.405 | 175 |
| Example 6-6 | BH1 | 3.055 | 0.510 | 0.16 | BD24 | 2.77 | 1.000 | 0.285 | 125 |
| Example 6-7 | BH2 | 3.090 | 0.180 | 0.605 | BD13 | 2.58 | 0.870 | 0.510 | 190 |
| Example 6-8 | BH2 | 3.090 | 0.180 | 0.605 | BD20 | 2.78 | 1.000 | 0.310 | 135 |
| Example 6-9 | BH2 | 3.090 | 0.180 | 0.605 | BD21 | 2.64 | 0.000 | 0.450 | 132 |
| Example 6-10 | BH2 | 3.090 | 0.180 | 0.605 | BD22 | 2.65 | 0.880 | 0.440 | 175 |
| Example 6-11 | BH2 | 3.090 | 0.180 | 0.605 | BD24 | 2.77 | 1.000 | 0.320 | 127 |
| Example 6-12 | BH3 | 2.960 | 0.170 | 0.857 | BD13 | 2.58 | 0.870 | 0.380 | 220 |
| Example 6-13 | BH3 | 2.960 | 0.170 | 0.857 | BD20 | 2.78 | 1.000 | 0.180 | 150 |
| Example 6-14 | BH3 | 2.960 | 0.170 | 0.857 | BD21 | 2.64 | 0.000 | 0.320 | 148 |
| Example 6-15 | BH3 | 2.960 | 0.170 | 0.857 | BD22 | 2.65 | 0.880 | 0.310 | 210 |
| Example 6-16 | BH3 | 2.960 | 0.170 | 0.857 | BD24 | 2.77 | 1.000 | 0.190 | 155 |
| Example 6-17 | BH4 | 3.003 | 0.930 | 0.951 | BD13 | 2.58 | 0.870 | 0.423 | 150 |
| Example 6-18 | BH4 | 3.003 | 0.930 | 0.951 | BD20 | 2.78 | 1.000 | 0.223 | 105 |
| Example 6-19 | BH4 | 3.003 | 0.930 | 0.951 | BD21 | 2.64 | 0.000 | 0.363 | 102 |
| Example 6-20 | BH4 | 3.003 | 0.930 | 0.951 | BD22 | 2.65 | 0.880 | 0.353 | 143 |
| Example 6-21 | BH4 | 3.003 | 0.930 | 0.951 | BD24 | 2.77 | 1.000 | 0.233 | 101 |
| Example 6-22 | BH11 | 2.940 | 0.720 | 0.75 | BD13 | 2.58 | 0.870 | 0.360 | 162 |
| Example 6-23 | BH11 | 2.940 | 0.720 | 0.75 | BD20 | 2.78 | 1.000 | 0.160 | 107 |
| Example 6-24 | BH11 | 2.940 | 0.720 | 0.75 | BD21 | 2.64 | 0.000 | 0.300 | 105 |
| Example 6-25 | BH11 | 2.940 | 0.720 | 0.75 | BD22 | 2.65 | 0.880 | 0.290 | 130 |
| Example 6-26 | BH11 | 2.940 | 0.720 | 0.75 | BD24 | 2.77 | 1.000 | 0.170 | 103 |
| Comparative | BH3 | 2.96 | 0.857 | 0.17 | BD4 | 2.87 | 1.000 | 0.09 | 70 |
| Example 6-1 | | | | | | | | | |
| Comparative | BH4 | 3.003 | 0.930 | 0.85 | BD4 | 2.87 | 1.000 | 0.133 | 67 |
| Example 6-2 | | | | | | | | | |
| Comparative | BH3 | 2.96 | 0.857 | 0.17 | BD14 | 2.81 | 0.000 | 0.15 | 74 |
| Example 6-3 | | | | | | | | | |
| Comparative | BHS | 2.962 | 0.855 | 1.19 | BD14 | 2.81 | 0.000 | 0.152 | 31 |
| Example 6-4 | | | | | | | | | |

As an electron transport material (ET), the following compounds were evaluated, and are shown in the following Table 7.

TABLE 7

| | |
|---|---|
| 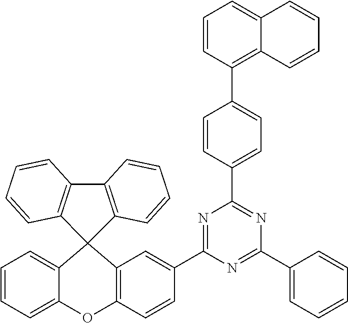 [SMILES: c1ccc(-c2nc(-c3ccc(-c4cccc5ccccc45)cc3)nc(-c3ccc4c(c3)C3(c5ccccc5O4)c4ccccc4-c4ccccc43)n2)cc1] | ETL1 |
| 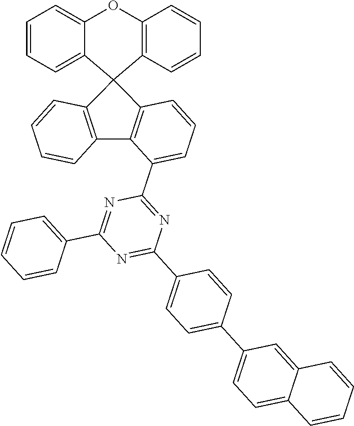 [SMILES: c1ccc(-c2nc(-c3ccc(-c4ccc5ccccc5c4)cc3)nc(-c3cccc4c3-c3ccccc3C43c4ccccc4Oc4ccccc43)n2)cc1] | ETL2 |
| 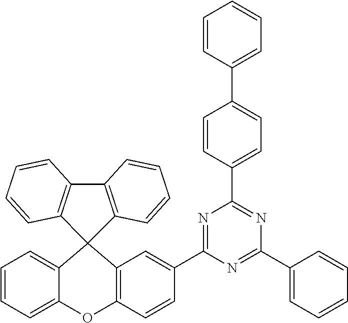 [SMILES: c1ccc(-c2ccc(-c3nc(-c4ccccc4)nc(-c4ccc5c(c4)C4(c6ccccc6O5)c5ccccc5-c5ccccc54)n3)cc2)cc1] | ETL3 |
| 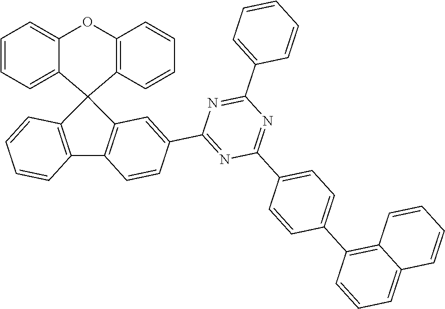 [SMILES: c1ccc(-c2nc(-c3ccc(-c4cccc5ccccc45)cc3)nc(-c3ccc4c(c3)C3(c5ccccc5Oc5ccccc53)c3ccccc3-4)n2)cc1] | ETL4 |
| 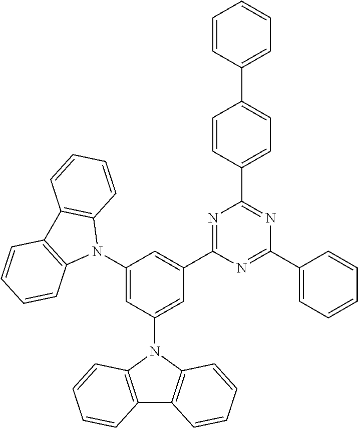 [SMILES: c1ccc(-c2ccc(-c3nc(-c4ccccc4)nc(-c4cc(-n5c6ccccc6c6ccccc65)cc(-n5c6ccccc6c6ccccc65)c4)n3)cc2)cc1] | ETL5 |
| 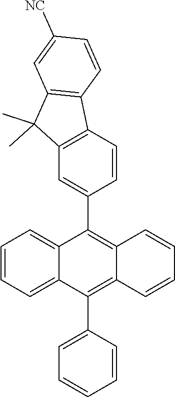 [SMILES: CC1(C)c2cc(C#N)ccc2-c2ccc(-c3c4ccccc4c(-c4ccccc4)c4ccccc34)cc21] | ETL6 |
| 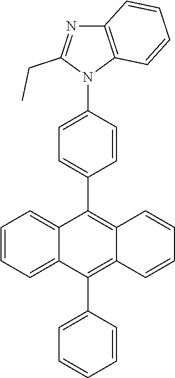 [SMILES: CCc1nc2ccccc2n1-c1ccc(-c2c3ccccc3c(-c3ccccc3)c3ccccc23)cc1] | ETL7 |

| | | Reduction | | |
|---|---|---|---|---|
| | | Iᵣ/I_{f} in 100 | | |
| | Compound | mV/s | AC3 LUMO (eV) | Device service life (%) |
| Example 7-1 | ELT1 | 0.98 | 2.7 | 93 |
| Example 7-2 | ETL2 | 0.97 | 2.87 | 139 |
| Example 7-3 | ETL3 | 0.96 | 2.72 | 91 |
| Example 7-4 | ETL4 | 0.95 | 2.82 | 120 |
| Example 7-5 | ELT5 | 0.72 | 2.9 | 90 |
| Comparative | ETL6 | 0.6 | 2.68 | 54 |
| Example 7-1 | | | | |
| Example 7-6 | ETL7 | 0.72 | 2.74 | 80 |

As a hole blocking material (HB), the following Compounds HB1 to HB7 were evaluated, and are shown in the following Table 8.

TABLE 8

| | |
|---|---|
| 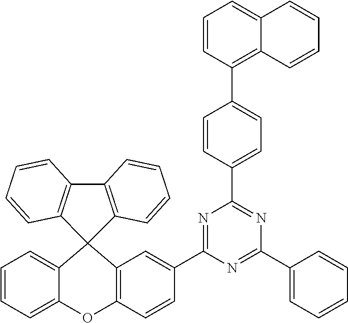 [SMILES: c1ccc(-c2nc(-c3ccc(-c4cccc5ccccc45)cc3)nc(-c3ccc4c(c3)C3(c5ccccc5O4)c4ccccc4-c4ccccc43)n2)cc1] | HB1 |
| 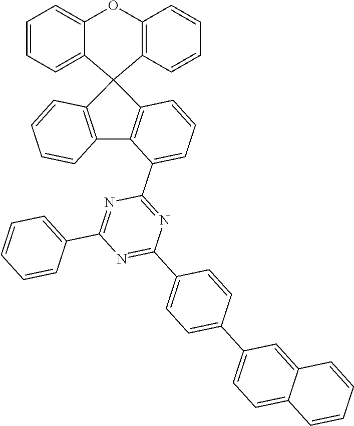 [SMILES: c1ccc(-c2nc(-c3ccc(-c4ccc5ccccc5c4)cc3)nc(-c3cccc4c3-c3ccccc3C43c4ccccc4Oc4ccccc43)n2)cc1] | HB2 |
| 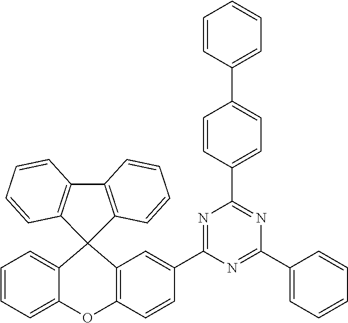 [SMILES: c1ccc(-c2ccc(-c3nc(-c4ccccc4)nc(-c4ccc5c(c4)C4(c6ccccc6O5)c5ccccc5-c5ccccc54)n3)cc2)cc1] | HB3 |
| 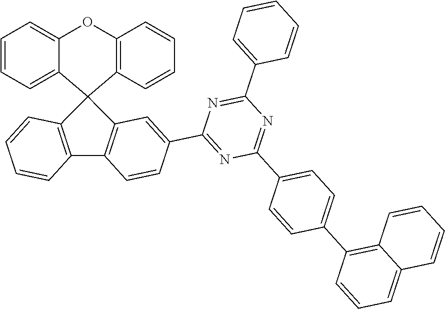 [SMILES: c1ccc(-c2nc(-c3ccc(-c4cccc5ccccc45)cc3)nc(-c3ccc4c(c3)C3(c5ccccc5Oc5ccccc53)c3ccccc3-4)n2)cc1] | HB4 |
| 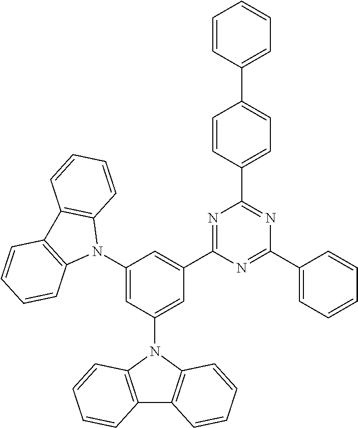 [SMILES: c1ccc(-c2ccc(-c3nc(-c4ccccc4)nc(-c4cc(-n5c6ccccc6c6ccccc65)cc(-n5c6ccccc6c6ccccc65)c4)n3)cc2)cc1] | HB5 |
| 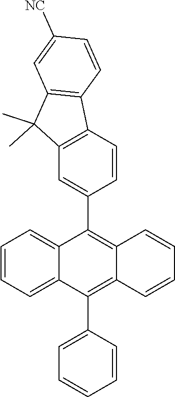 [SMILES: CC1(C)c2cc(C#N)ccc2-c2ccc(-c3c4ccccc4c(-c4ccccc4)c4ccccc34)cc21] | HB6 |
| 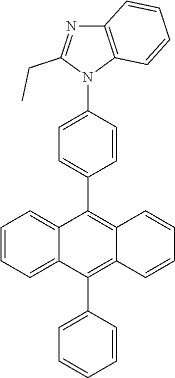 [SMILES: CCc1nc2ccccc2n1-c1ccc(-c2c3ccccc3c(-c3ccccc3)c3ccccc23)cc1] | HB7 |

| | Compound | LUMO (eV) | Oxidation stability in 300 mV/s | Device service life (%) |
|---|---|---|---|---|
| Comparative | HB1 | 2.7 | 0 | 87 |
| Example 8-1 | | | | |
| Comparative | HB2 | 2.87 | 0 | 51 |
| Example 8-2 | | | | |
| Comparative | HB3 | 2.72 | 0 | 100 |
| Example 8-3 | | | | |
| Comparative | HB4 | 2.82 | 0 | 57 |
| Example 8-4 | | | | |
| Comparative | HB5 | 2.9 | 0 | 105 |
| Example 8-5 | | | | |
| Example 8-1 | HB6 | 2.68 | 0.66 | 140 |
| Example 8-2 | HB7 | 2.74 | 0.94 | 182 |

The service life of each of the devices including both the electron transport material (ET) and the hole blocking material (HB) was measured, and is shown in the following Table 9. “LUNO difference” in the following Table 9 refers to the value of (the LUNO of the electron transport material (ET)−the LUNO of the hole blocking material (HB)).

TABLE 9

| | Hole blocking material (HB) | | | Electron transport material (ET) | | | | |
|---|---|---|---|---|---|---|---|---|
| | | Oxidation | | | Reduction | | Device | |
| | | Iᵣ/I_{f} | | | Iᵣ/I_{f} | | | Service |
| | | in 100 | AC3 | | in 100 | AC3 | LUMO | life |
| | Compound | mV/s | LUMO | Compound | mV/s | LUMO | Difference | (%) |
| Example 9-1 | HB6 | 0.66 | 2.68 | ETL2 | 0.97 | 2.87 | 0.19 | 132 |
| Example 9-2 | HB6 | 0.66 | 2.68 | ETL4 | 0.95 | 2.82 | 0.14 | 120 |
| Example 9-3 | HB7 | 0.94 | 2.74 | ETL2 | 0.97 | 2.87 | 0.13 | 170 |
| Example 9-4 | HB7 | 0.94 | 2.74 | ETL4 | 0.95 | 2.82 | 0.08 | 150 |
| Example 9-5 | HB7 | 0.94 | 2.74 | ETL5 | 0.72 | 2.90 | 0.16 | 140 |
| Comparative | HB4 | 0.00 | 2.82 | ETL3 | 0.96 | 2.72 | −0.1 | 65 |
| Example 9-1 | | | | | | | | |
| Example 9-6 | HB6 | 0.66 | 2.68 | ETL1 | 0.98 | 2.70 | 0.02 | 95 |

As a light emitting host material (EML), the following compounds were evaluated, and shown in the following Table 10. Q2 in the following Table 10 is a value of {[the LUMO absolute value of the light emitting host material (EML)]−[the LUMO absolute value of the electron transport material (ET)].

TABLE 10

| | |
|---|---|
| 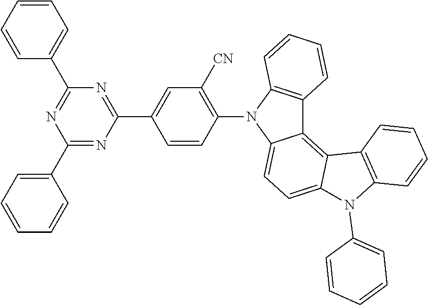 [SMILES: N#Cc1cc(-c2nc(-c3ccccc3)nc(-c3ccccc3)n2)ccc1-n1c2ccccc2c2c3c4ccccc4n(-c4ccccc4)c3ccc21] | EML1 |
| 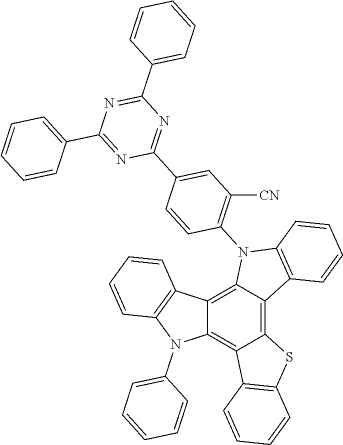 [SMILES: N#Cc1cc(-c2nc(-c3ccccc3)nc(-c3ccccc3)n2)ccc1-n1c2ccccc2c2c3sc4ccccc4c3c3c(c4ccccc4n3-c3ccccc3)c21] | EML2 |
| 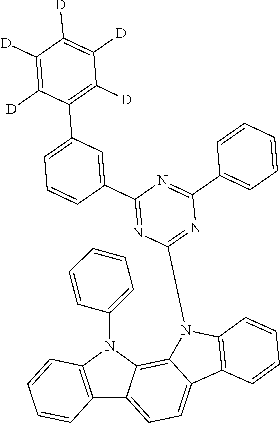 [SMILES: [2H]c1c([2H])c([2H])c(-c2cccc(-c3nc(-c4ccccc4)nc(-n4c5ccccc5c5ccc6c7ccccc7n(-c7ccccc7)c6c54)n3)c2)c([2H])c1[2H]] | EML3 |
| 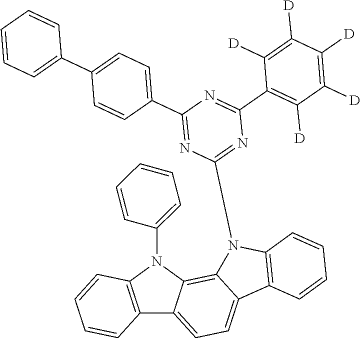 [SMILES: [2H]c1c([2H])c([2H])c(-c2nc(-c3ccc(-c4ccccc4)cc3)nc(-n3c4ccccc4c4ccc5c6ccccc6n(-c6ccccc6)c5c43)n2)c([2H])c1[2H]] | EML4 |
| 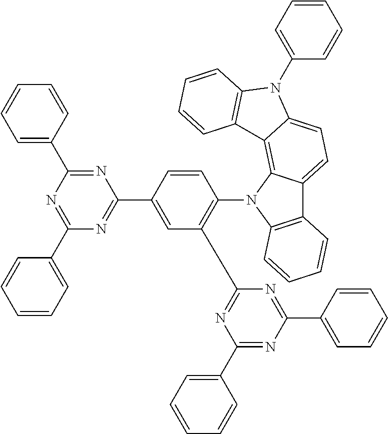 [SMILES: c1ccc(-c2nc(-c3ccccc3)nc(-c3ccc(-n4c5ccccc5c5ccc6c(c7ccccc7n6-c6ccccc6)c54)c(-c4nc(-c5ccccc5)nc(-c5ccccc5)n4)c3)n2)cc1] | EML5 |
| 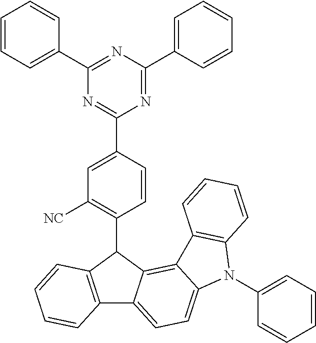 [SMILES: N#Cc1cc(-c2nc(-c3ccccc3)nc(-c3ccccc3)n2)ccc1C1c2ccccc2-c2ccc3c(c21)c1ccccc1n3-c1ccccc1] | EML6 |
| 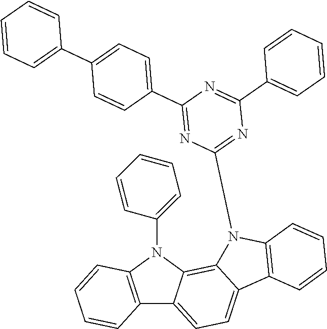 [SMILES: c1ccc(-c2ccc(-c3nc(-c4ccccc4)nc(-n4c5ccccc5c5ccc6c7ccccc7n(-c7ccccc7)c6c54)n3)cc2)cc1] | EML7 |
| 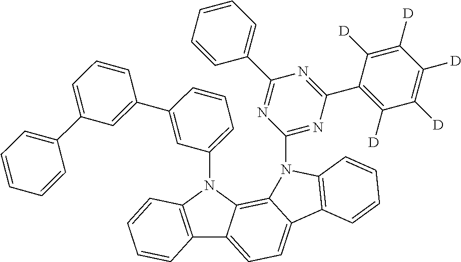 [SMILES: [2H]c1c([2H])c([2H])c(-c2nc(-c3ccccc3)nc(-n3c4ccccc4c4ccc5c6ccccc6n(-c6cccc(-c7cccc(-c8ccccc8)c7)c6)c5c43)n2)c([2H])c1[2H]] | EML8 |
| 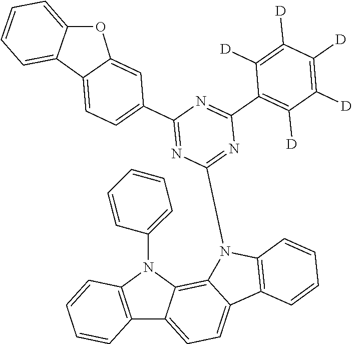 [SMILES: [2H]c1c([2H])c([2H])c(-c2nc(-c3ccc4c(c3)oc3ccccc34)nc(-n3c4ccccc4c4ccc5c6ccccc6n(-c6ccccc6)c5c43)n2)c([2H])c1[2H]] | EML9 |
| 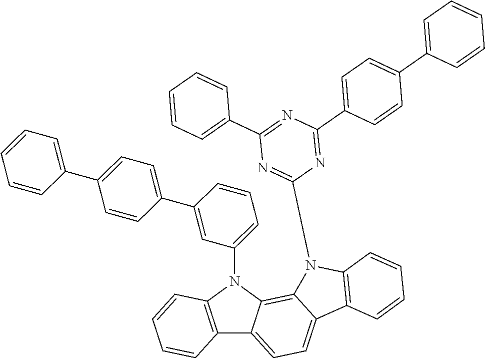 [SMILES: c1ccc(-c2ccc(-c3cccc(-n4c5ccccc5c5ccc6c7ccccc7n(-c7nc(-c8ccccc8)nc(-c8ccc(-c9ccccc9)cc8)n7)c6c54)c3)cc2)cc1] | EML10 |
| 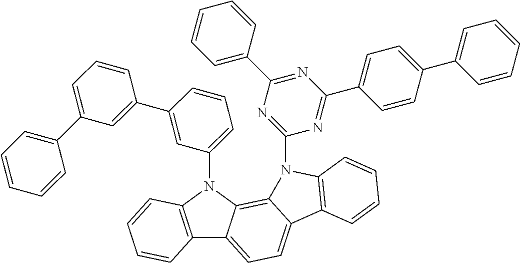 [SMILES: c1ccc(-c2ccc(-c3nc(-c4ccccc4)nc(-n4c5ccccc5c5ccc6c7ccccc7n(-c7cccc(-c8cccc(-c9ccccc9)c8)c7)c6c54)n3)cc2)cc1] | EML11 |

| | | | | Oxidation | Reduction | | |
|---|---|---|---|---|---|---|---|
| | | | | Iᵣ/I_{f} | Iᵣ/I_{f} | Service | |
| | | AC3 | | in 100 | in 10 | life | |
| | Compound | HOMO | LUMO | mV/s | mV/s | (%) | Q2 |
| Example 10-1 | EML1 | 5.63 | 3.01 | 0.00 | 0.96 | 85 | 0.955 |
| Example 10-2 | EML2 | 5.74 | 3.056 | 0.00 | 0.98 | 100 | 0.955 |
| Example 10-3 | EML3 | 5.6 | 3.058 | 1.00 | 0.93 | 263 | 0.7764 |
| Comparative | EML4 | 5.67 | 3.058 | 1.00 | 0.71 | 5 | 0.7764 |
| Example 10-1 | | | | | | | |
| Example 10-4 | EML5 | 5.97 | 2.89 | 0 | 0.96 | 82 | 0.955 |
| Comparative | EML6 | 5.9 | 2.83 | 0 | 0.94 | 69 | 0.955 |
| Example 10-2 | | | | | | | |
| Example 10-5 | EML7 | 5.83 | 2.754 | 0 | 0.98 | 107 | 0.955 |
| Example 10-6 | EML8 | 5.87 | 2.78 | 0 | 0.97 | 101 | 0.955 |
| Example 10-7 | EML9 | 5.85 | 2.94 | 0 | 0.99 | 125 | 0.955 |
| Example 10-8 | EML10 | 5.92 | 2.86 | 0 | 1.00 | 114 | 0.955 |
| Example 10-9 | EML11 | 5.83 | 2.76 | 0 | 1.01 | 130 | 0.955 |

The service life of each of the devices including both the light emitting host material (EML) and the electron transport material (ET) was measured, and is shown in the following Table 11. “LUNO difference” in the following Table 11 refers to a value of (the LUNO of the light emitting host material (EML)−the LUNO of the electron transport material (ET)).

TABLE 11

| | | | Electron | | | |
|---|---|---|---|---|---|---|
| | Light emitting | | transport | | | |
| | host material | | material | | Device | |
| | (EML) | | (ET) | | LUMO | Service |
| | Com- | AC3 | Com- | AC3 | dif- | life |
| | pound | LUMO | pound | LUMO | ference | (%) |
| Example 11-1 | EML2 | 3.056 | ETL2 | 2.87 | 0.186 | 120 |
| Example 11-2 | EML3 | 3.058 | ETL2 | 2.87 | 0.188 | 280 |
| Example 11-3 | EML7 | 2.754 | ETL2 | 2.87 | −0.116 | 125 |
| Example 11-4 | EML10 | 2.86 | ETL2 | 2.87 | −0.01 | 140 |
| Example 11-5 | EML11 | 2.76 | ETL2 | 2.87 | −0.11 | 150 |
| Comparative | EML1 | 3.01 | ETL2 | 2.87 | 0.14 | 89 |
| Example 11-1 | | | | | | |
| Example 11-6 | EML2 | 3.056 | ETL3 | 2.72 | 0.336 | 100 |
| Example 11-7 | EML3 | 3.058 | ETL3 | 2.72 | 0.338 | 252 |
| Example 11-8 | EML7 | 2.754 | ETL3 | 2.72 | 0.034 | 110 |
| Example 11-9 | EML10 | 2.86 | ETL3 | 2.72 | 0.14 | 115 |
| Example 11-10 | EML11 | 2.76 | ETL3 | 2.72 | 0.04 | 125 |
| Comparative | EML1 | 3.01 | ETL3 | 2.72 | 0.29 | 80 |
| Example 11-2 | | | | | | |
| Example 11-11 | EML2 | 3.056 | ETL4 | 2.82 | 0.236 | 110 |
| Example 11-12 | EML3 | 3.058 | ETL4 | 2.82 | 0.238 | 270 |
| Example 11-13 | EML7 | 2.754 | ETL4 | 2.82 | −0.066 | 117 |
| Example 11-14 | EML10 | 2.86 | ETL4 | 2.82 | 0.04 | 120 |
| Example 11-15 | EML11 | 2.76 | ETL4 | 2.82 | −0.06 | 142 |
| Comparative | EML1 | 3.01 | ETL4 | 2.82 | 0.19 | 87 |
| Example 11-3 | | | | | | |
| Example 11-16 | EML7 | 2.754 | ETL1 | 2.7 | 0.054 | 104 |
| Example 11-17 | EML7 | 2.754 | ETL3 | 2.72 | 0.034 | 110 |
| Comparative | EML7 | 2.754 | ETL5 | 2.90 | −0.146 | 89 |
| Example 11-4 | | | | | | |

Through the Examples, it can be seen that an organic light emitting device including a compound having CV characteristics according to the present invention has long service life characteristics.

1. An organic light emitting device, comprising:
a positive electrode;
a negative electrode; and
an organic material layer provided between the positive electrode and the negative electrode,
wherein the organic material layer comprises a hole transport material (HT),
wherein the organic material layer comprises an electron blocking material (EB), and
the hole transport material (HT) has a HOMO absolute value of 4.30 eV to 4.60 eV, and a reversibility value (Iᵣ/I_{f}) of 0.83 or higher within an oxidation range at a scan rate of 100 mV/s at the time of measuring cyclic voltage current,
the electron blocking material (EB) has a reversibility value (Iᵣ/I_{f}) of more than 0.5 within an oxidation range at a scan rate of 100 mV/s at the time of measuring cyclic voltage current.

2. (canceled)

3. An organic light emitting device comprising:
a positive electrode;
a negative electrode; and
an organic material layer provided between the positive electrode and the negative electrode,
wherein the organic material layer comprises a blue light emitting dopant material (BD),
wherein the organic material layer comprises a blue light emitting host material (BH), and
the blue light emitting dopant material (BD) has a LUMO absolute value of 2.40 eV to 2.74 eV, and a reversibility value (Iᵣ/I_{f}) larger than [−23.14+8.458×(the LUMO absolute value)] within a reduction range at a scan rate of 100 mV/s at the time of measuring cyclic voltage current,
the blue light emitting host material (BH) has a reversibility value (Iᵣ/I_{f}) of [1.34×(the dipole moment)−0.293] or higher within an oxidation range at a scan rate of 500 mV/s and a reversibility value (Iᵣ/I_{f}) of 0.95 or higher within a reduction range at a scan rate of 10 mV/s, at the time of measuring cyclic voltage current.

4. An organic light emitting device comprising:
a positive electrode;
a negative electrode; and
an organic material layer provided between the positive electrode and the negative electrode,
wherein the organic material layer comprises an electron transport material (ET),
wherein the organic material layer comprises a hole blocking material (HB), and
the electron transport material (ET) has a LUMO absolute value of 2.60 eV to 2.90 eV, and a reversibility value (Iᵣ/I_{f}) larger than [4.96−1.535×(the LUMO absolute value)] within a reduction range at a scan rate of 100 mV/s at the time of measuring cyclic voltage current,
the hole blocking material (HB) has both a forward peak and an inverse peak at a scan rate of 100 mV/s at the time of measuring cyclic voltage current within an oxidation range.

5.-6. (canceled)

7. An organic light emitting device comprising:
a positive electrode;
a negative electrode; and
an organic material layer provided between the positive electrode and the negative electrode,
wherein the organic material layer comprises a light emitting host material (EML),
wherein the organic material layer comprises an electron transport material (ET), and
the light emitting host material (EML) has a reversibility value (Iᵣ/I_{f}) of [0.955−0.1786×(a reversibility value (Iᵣ/I_{f}) within an oxidation range)] or higher within a reduction range at a scan rate of 10 mV/s at the time of measuring cyclic voltage current,
the electron transport material (ET) has a LUMO absolute value of 2.60 eV to 2.90 eV, and a reversibility value (Iᵣ/I_{f}) larger than [4.96−1.535×(the LUMO absolute value)] within a reduction range at a scan rate of 100 mV/s at the time of measuring cyclic voltage current.

8. The organic light emitting device of claim 1, wherein
a value of (HT Iᵣ/I_{f})−(EB Iᵣ/I_{f}) is 0.15 or lower,
the HT Iᵣ/I_{f} is a reversibility value of the hole transport material (HT) within an oxidation range at a scan rate of 100 mV/s, and
the EB Iᵣ/I_{f} is a reversibility value of the electron blocking material (EB) within an oxidation range at a scan rate of 100 mV/s.

9. The organic light emitting device of claim 3, wherein
a value of {[the LUMO absolute value of the blue light emitting host material (BH)]−[the LUMO absolute value of the blue light emitting dopant material (BD)]} is 0.16 eV to 0.75 eV.

10. The organic light emitting device of claim 4, wherein
[the LUMO absolute value of the electron transport material (ET)−the LUMO absolute value of the hole blocking material (HB)] is 0.05 eV to 0.3 eV.

11. The organic light emitting device of claim 7, wherein
a value of {[the LUMO absolute value of the light emitting host material (EML)]−[the LUMO absolute value of the electron transport material (ET)]} is 0.15 eV to 0.35 eV.

12. The organic light emitting device of claim 1, wherein the hole transport material (HT) is a compound of the following Formula 1 or 2: 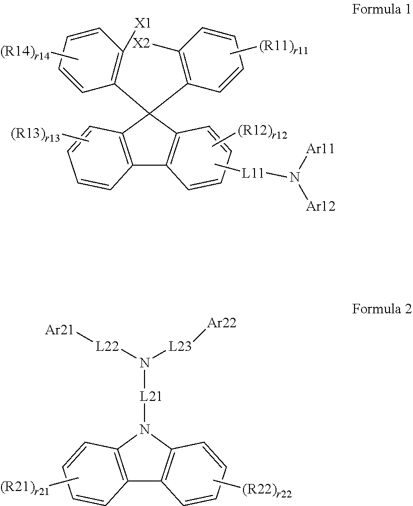
wherein in Formulae 1 and 2:
X1 and X2 are each hydrogen or deuterium, or are directly single-bonded to each other to form a ring;
R11 to R14, R21 and R22 are the same as or different from each other, and are each independently hydrogen, deuterium, a substituted or unsubstituted alkyl group, a substituted or unsubstituted amine group, or a substituted or unsubstituted aryl group, or are optionally bonded to an adjacent group to form a substituted or unsubstituted ring;
L11 and L21 to L23 are the same as or different from each other, and are each independently a single bond or a substituted or unsubstituted arylene group;
Ar11, Ar12, Ar21 and Ar22 are the same as or different from each other, and are each independently hydrogen, deuterium, a halogen group, a cyano group, a nitro group, a substituted or unsubstituted silyl group, a substituted or unsubstituted cycloalkyl group, a substituted or unsubstituted aryl group, or a substituted or unsubstituted heteroaryl group;
r11, r13, r14, r21, and r22 are each an integer from 0 to 4, and r12 is an integer from 0 to 3; and
when r11 to r14, r21, and r22 are 2 or higher, the substituents in the parenthesis are the same as or different from each other.

13. The organic light emitting device of claim 1, wherein the electron blocking material (EB) is a compound of the following Formula 1 or 2: 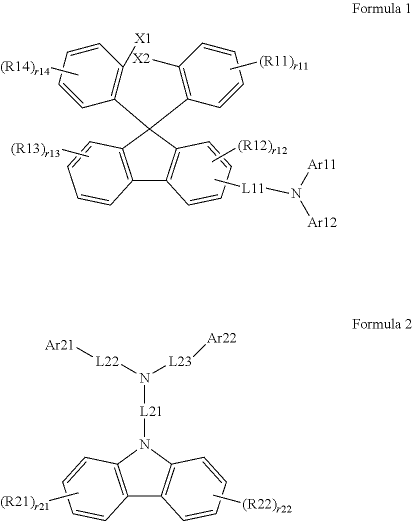
wherein in Formulae 1 and 2:
X1 and X2 are each hydrogen or deuterium, or are directly single-bonded to each other to form a ring;
R11 to R14, R21 and R22 are the same as or different from each other, and are each independently hydrogen, deuterium, a substituted or unsubstituted alkyl group, a substituted or unsubstituted amine group, or a substituted or unsubstituted aryl group, or are optionally bonded to an adjacent group to form a substituted or unsubstituted ring;
L11 and L21 to L23 are the same as or different from each other, and are each independently a single bond or a substituted or unsubstituted arylene group;
Ar11, Ar12, Ar21 and Ar22 are the same as or different from each other, and are each independently hydrogen, deuterium, a halogen group, a cyano group, a nitro group, a substituted or unsubstituted silyl group, a substituted or unsubstituted cycloalkyl group, a substituted or unsubstituted aryl group; or a substituted or unsubstituted heteroaryl group;
r11, r13, r14, r21, and r22 are each an integer from 0 to 4, and r12 is an integer from 0 to 3; and
when r11 to r14, r21, and r22 are 2 or higher, the substituents in the parenthesis are the same as or different from each other.

14. The organic light emitting device of claim 3, wherein the blue light emitting dopant material (BD) is a compound of any one of the following Formulae 3 to 6: 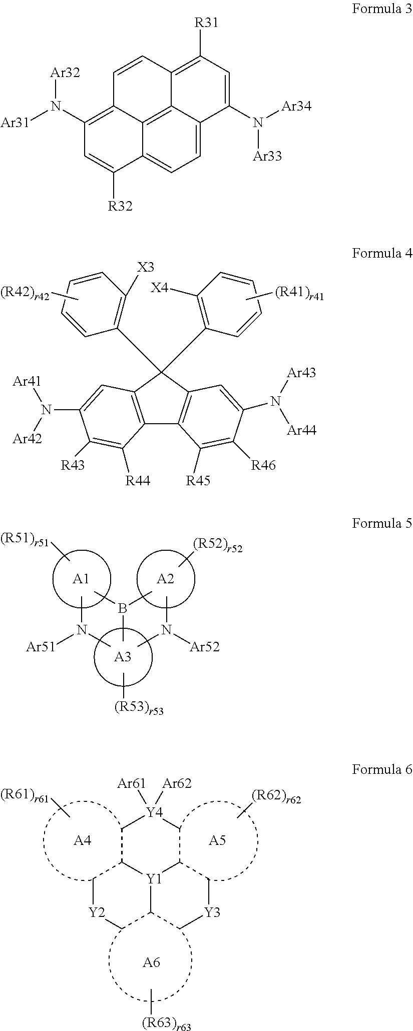
wherein in Formulae 3 to 6:
R31 and R32 are the same as or different from each other, and are each independently hydrogen, deuterium, a substituted or unsubstituted alkyl group, a substituted or unsubstituted silyl group, or a substituted or unsubstituted aryl group;
X3 and X4 are each hydrogen or deuterium, or are directly single-bonded to each other to form a ring;
R41 and R42 are the same as or different from each other, and are each independently hydrogen, deuterium, or a substituted or unsubstituted alkyl group;
R43 to R46 are the same as or different from each other, and are each independently hydrogen, deuterium, a substituted or unsubstituted alkyl group, or a substituted or unsubstituted aryl group, or adjacent substituents are bonded to each other to form a substituted or unsubstituted ring;
Ar31 to Ar34 and Ar41 to Ar44 are the same as or different from each other, and are each independently a substituted or unsubstituted aryl group or substituted or unsubstituted heteroaryl group;
A1 to A6 are the same as or different from each other, and are each independently a monocyclic to polycyclic aromatic hydrocarbon ring or monocyclic to polycyclic aromatic hetero ring;
R51 to R53 and R61 to R63 are the same as or different from each other, and are each independently hydrogen, deuterium, a substituted or unsubstituted alkyl group, a substituted or unsubstituted silyl group, a substituted or unsubstituted amine group, a substituted or unsubstituted aryl group, or a substituted or unsubstituted heteroaryl group, or adjacent substituents are bonded to each other to form a substituted or unsubstituted aromatic ring or a substituted or unsubstituted aliphatic ring;
Y1 is B or N;
Y2 is O, S, or N(Ar63)(Ar64);
Y3 is O, S, or N(Ar65)(Ar66);
Y4 is C or Si;
Ar51, Ar52, and Ar61 to Ar66 are the same as or different from each other, and are each independently a substituted or unsubstituted alkyl group, a substituted or unsubstituted aryl group, or a substituted or unsubstituted heteroaryl group, or are bonded to an adjacent substituent to form a substituted or unsubstituted aromatic ring or a substituted or unsubstituted aliphatic ring; and
r41, r42, r51 to r53, and r61 to r63 are each an integer from 0 to 4, and when r41, r42, r51 to r53, and r61 to r63 are 2 or higher, substituents in the parenthesis are the same as or different from each other.

15. The organic light emitting device of claim 4, wherein the electron transport material (ET) is of the following Formula 8: 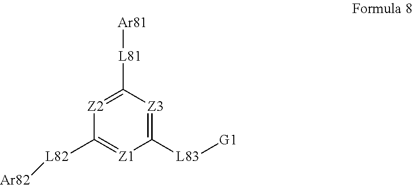
wherein in Formula 8:
at least one of Z1 to Z3 is N, and the others are CH,
L81 to L83 are the same as or different from each other, and are each independently a direct bond, a substituted or unsubstituted arylene group, or a substituted or unsubstituted heteroarylene group;
Ar81 and Ar82 are the same as or different from each other, and are each independently a substituted or unsubstituted aryl group or a substituted or unsubstituted heteroaryl group;
G1 is a monovalent substituent of any one of the following Formulae 801 to 804: 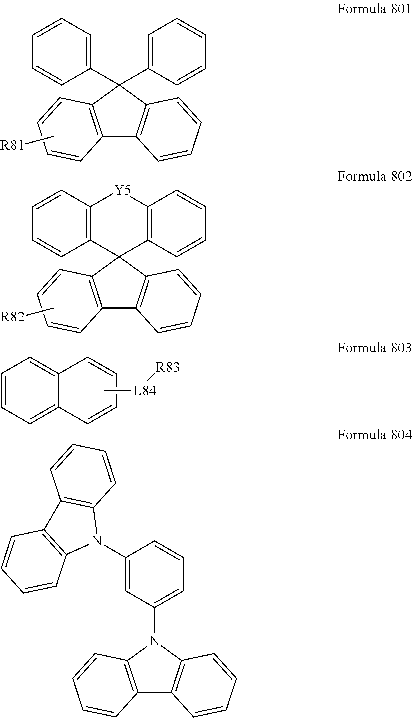
wherein in Formulae 801 to 80:
any one carbon is linked to L83 of Formula 8;
Y5 is O or S;
L84 is a substituted or unsubstituted arylene group; and
R81 to R83 are the same as or different from each other, and are each independently hydrogen, deuterium, a cyano group, an aryl group, or an aryl group which is substituted with a cyno group.

16. The organic light emitting device of claim 4, wherein the hole blocking material (HB) is of the following Formula 9: 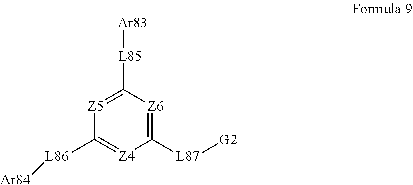
wherein in Formula 9:
at least one of Z4 to Z6 is N, and the others are CH;
L85 to L87 are the same as or different from each other, and are each independently a direct bond, a substituted or unsubstituted arylene group, or a substituted or unsubstituted heteroarylene group;
Ar83 and Ar84 are the same as or different from each other, and are each independently a substituted or unsubstituted aryl group or a substituted or unsubstituted heteroaryl group;
G2 is a monovalent substituent of the following Formula 901: 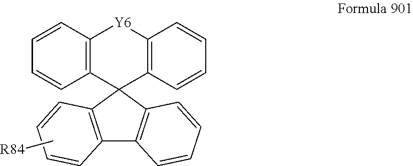
wherein in Formula 901:
any one carbon is linked to L87 of Formula 9;
Y6 is O or S; and
R84 is hydrogen, deuterium, a cyano group, an aryl group, or an aryl group which is substituted with a cyano group.

17. The organic light emitting device of claim 3, wherein the blue light emitting host material (BH) is of the following Formula H: 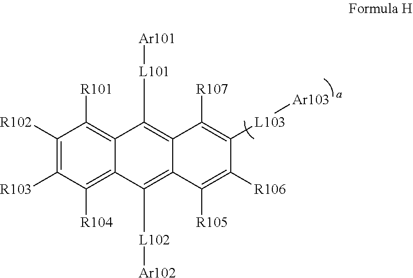
wherein in Formula H:
L101 to L103 are the same as or different from each other, and are each independently a direct bond, a substituted or unsubstituted arylene group, or a substituted or unsubstituted heteroarylene group;
R101 to R107 are the same as or different from each other, and are each independently hydrogen, deuterium, a substituted or unsubstituted alkyl group, a substituted or unsubstituted cycloalkyl group, a substituted or unsubstituted silyl group, a substituted or unsubstituted aryl group, or a substituted or unsubstituted heteroaryl group;
Ar101 to Ar103 are the same as or different from each other, and are each independently a substituted or unsubstituted aryl group or a substituted or unsubstituted heteroaryl group; and
a is 0 or 1.

18. The organic light emitting device of claim 7, wherein the light emitting host material (EML) is of the following Formula 10: 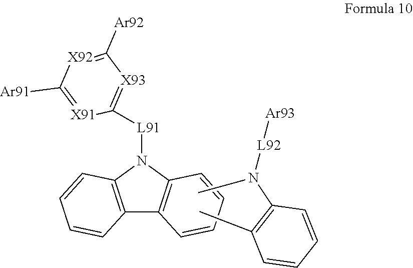
wherein in Formula 10:
at least one of X91 to X93 is N, and the others are CH;
L91 and L92 are the same as or different from each other, and are each independently a direct bond or a substituted or unsubstituted arylene group; and
Ar91 to Ar93 are the same as or different from each other, and are each independently a substituted or unsubstituted aryl group or a substituted or unsubstituted heteroaryl group.

19. The organic light emitting device of claim 4, wherein the hole blocking material (HB) is of the following Formula 11 or 12: 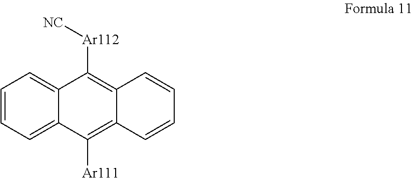
wherein in Formula 11:
Ar111 is a substituted or unsubstituted aryl group or a substituted or unsubstituted heteroaryl group; and
Ar112 is a substituted or unsubstituted arylene group or a substituted or unsubstituted heteroarylene group, 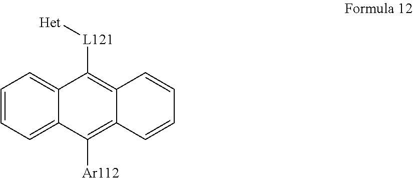
wherein in Formula 12:
Het is a substituted or unsubstituted N-containing heteroaryl group;
Ar112 is a substituted or unsubstituted aryl group or a substituted or unsubstituted aryl group; and
L121 is a substituted or unsubstituted arylene group or a substituted or unsubstituted heteroarylene group.
